(19) United States
(12) Patent Application Publication
(10) Pub. No.: US 2007/0048791 A1
(43) Pub. Date: Mar. 1, 2007

(54) CRYSTAL STRUCTURES AND MODELS FOR Fc RECEPTORS AND USES THEREOF IN THE DESIGN OR IDENTIFICATION OF Fc RECEPTOR MODULATOR COMPOUNDS

(75) Inventors: Geoffrey Allan Pietersz, Greensborough (AU); Tessa Margaret Bradford, Parkside (AU); Phillip Mark Hogarth, Williamstown (AU); Bruce David Wines, Heidelberg (AU); Paul Allen Ramsland, Bentleigh East (AU)

Correspondence Address: SHERIDAN ROSS PC, 1560 BROADWAY, SUITE 1200, DENVER, CO, 80202, US

(73) Assignee: The Macfarlane Burnet Institute for Medical Research and Public Health Ltd., Melbourne (AU)

(21) Appl. No.: 11/463,552

(22) Filed: Aug. 9, 2006

Related U.S. Application Data
(63) Continuation-in-part of application No. PCT/AU05/00176, filed on Feb. 10, 2005.

(30) Foreign Application Priority Data
Feb. 10, 2004 (AU) 20040900615

(51) Int. Cl. G01N 33/53; G06F 19/00

(52) U.S. Cl. 435/7.1; 702/19

(57) ABSTRACT

The invention relates to the determination of the three-dimensional structures of Fc receptor proteins, particularly wild-type FcγRIIa, by X-ray crystallography and the use of the structure in identifying and modifying agents for modulating the biological activity of Fc receptors. Also disclosed is a novel dimeric structure for FcγRIIa and novel target sites for agents for modulating the biological activity of Fc receptor proteins.

### CROSS-REFERENCE TO RELATED APPLICATIONS

This application is a Continuation-In-Part of PCT/AU2005/000176 filed on Feb. 10, 2005, which claims priority to AU20040900615 filed on Feb. 10, 2004, each of which is hereby incorporated by reference for all purposes.

### FIELD OF THE INVENTION

The present invention relates to the determination of the three-dimensional structures of Fc receptor proteins, particularly wild-type FcγRIIa, by X-ray crystallography and the use of said structure in identifying and modifying agents for modulating the biological activity of Fc receptors.

### BACKGROUND OF THE INVENTION

Interactions between the various classes of antibodies and Fc receptors (FcR) initiate a wide range of immunological responses. These include antibody-specific antigen uptake for presentation of MHC bound peptides to T cells, degranulation of mast cells in allergy, and immune complex mediated hypersensitivity and inflammation. The FcR have also been shown to function as recognition molecules for viral infections in measles and Dengue fever. In humans, the most prevalent and abundant IgG FcR is designated as FcγRIIa or CD32. Repeated triggering of FcγRIIa by immune complexes is a major pathway resulting in the chronic and acute episodes of inflammation associated with antibody-mediated autoimmune diseases like systemic lupus erythematosus (SLE) and rheumatoid arthritis (reviewed in Hogarth, 2002).

Human FcγRIIa exists as two predominant alleles classified as the low responder (LR) and the high responder (HR) wild-type polymorphisms. At the level of protein sequence the difference is that the LR receptor has a histidine (H) while the HR receptor has an arginine (R) residue at position 134 (often designated in the literature as position 131) in the amino acid sequence (Warmerdam et al, 1990). The differences between the LR and HR FcγRIIa alleles relate to their different abilities to bind mouse IgG1 and human IgG2 (Sautes et al, 1991; Parren et al, 1992). Genetic polymorphisms of the FcγR have been shown to be linked to susceptibility in inflammatory diseases like the rheumatic diseases and efficacy of antibody dependent cellular cytotoxicity (ADCC) in the clinical assessment of therapeutic antibodies (Weng and Levy, 2003).

In contrast to all other activating FcR molecules, the signalling ITAM (immunoreceptor tyrosine-based activation motif) is located within the cytoplasmic tail of FcγRIIa. Other activating FcR molecules associate with ITAM-containing accessory molecules, which mediate the intracellular aspects of the signalling event (Hogarth, 2002). The crystal structure of the LR allele of the FcγRIIa glycoprotein was reported to have a major crystallographic dimer formed around a twofold axis in the P2₁2₁2 crystals (Maxwell et al, 1999). Such an arrangement brings two ITAM-containing cytoplasmic tails of FcγRIIa into close proximity. Another crystal structure has been reported for a non-glycosylated (E. coli-derived) form of the HR allele of FcγRIIa from C2 crystals, which the authors outlined did not form the same dimer as was reported for the glycosylated LR allele of FcγRIIa (Sondermann et al, 2001).

In the LR FcγRIIa crystal structure described by Maxwell et al (1992), there was an introduced point mutation in the original cloning of the LR FcγRIIa cDNA used to generate the P2₁2₁2 crystals. The mutation was of a serine to phenylalanine at position 88 of the LR FcγRIIa gene. The LR mutant is hereinafter referred to as LR₁₋₈₈. The LR wild-type is hereinafter referred to as LR_{S88} and the HR wild-type is hereinafter referred to as HR_{S88}.

The process of rational or structure-based drug design requires no explanation or teaching for the person skilled in the art, but a brief description is given here of computational design for the lay reader. The person skilled in the art may use one of several methods to screen chemical entities or fragments for their ability to associate with a target molecule. For example, the screening process may begin by visual inspection of the target molecule, or a portion thereof, on a computer screen, generated from a machine-readable storage medium. Selected fragments or chemical entities may then be positioned in a variety of orientations, or docked, within identified or possible binding pockets (ie target sites). Docking may be accomplished using software such as Quanta (Accelrys, Inc, Burlington, Mass., USA) and Sybyl (Tripos Associates, St Louis, Mo., USA) followed by energy minimisation and molecular dynamics with standard molecular mechanics force fields, such as CHARMM (Accelrys, Inc, Burlington, Mass., USA) and AMBER (Weiner et al, 1984; Kollman, Pa., University of California, San Francisco, Calif., USA).

Specialised computer programs may also assist in the process of selecting fragments or chemical entities. These include:
1. GRID (Goodford, 1985). GRID is available from Oxford University, Oxford, UK.
2. MCSS (Miranker, 1991). MCSS is available from Accelrys, Inc, Burlington, Mass., USA.
3. AUTODOCK (Goodsell, 1990). AUTODOCK is available from Scripps Research Institute, La Jolla, Calif., USA.
4. DOCK (Kuntz, 1982). DOCK is available from University of California, San Francisco, Calif., USA.

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of the target molecule. This is generally followed by manual model building using software such as Quanta or Sybyl.

Useful programs to aid the person skilled in the art in connecting the individual chemical entities or fragments include:
1. CAVEAT (Bartlett et al 1989). CAVEAT is available from the University of California, Berkeley, Calif., USA.
2. 3D Database systems such as MACCS-3D (MDL Information Systems, San Leandro, Calif., USA). This area is reviewed in Martin, 1992.
3. HOOK (available from Accelrys, Inc, Burlington, Mass., USA).

As is well known to the person skilled in the art, instead of proceeding to build a single compound or complex for the target site in a step-wise fashion, one fragment or chemical entity at a time as described above, inhibitory or other target-binding compounds may be designed as a whole or de novo. Methods for achieving such include:
1. LUDI (Bohm, 1992). LUDI is available from Accelrys, Inc, Burlington, Mass., USA.
2. LEGEND (Nishibata, 1991). LEGEND is available from Accelrys, Inc, Burlington, Mass., USA.
3. LeapFrog (Tripos Associates, St Louis, Mo., USA).

Other molecular modelling techniques may also be employed, see for example, Cohen, 1990 and Navia, et al, Current Opinion in Structural Biology, 2: 202-210, 1992).

Once a single compound or chemical complex has been designed or selected by the above methods, the efficiency with which that entity may bind to a target site may be tested and optimised by computational evaluation. For example, an effective entity will preferably demonstrate a relatively small difference in energy between its bound and free states (ie a small deformation energy of binding). Thus, the most efficient entities should preferably be designed with a deformation energy of binding of not greater than about 10 kcal/mole, and preferably, not greater than 7 kcal/mole. Further, some entities may interact with the target site in more than one conformation that is similar in overall binding energy. In those cases, the deformation energy of binding is taken to be the difference between the energy of the free entity and the average energy of the conformations observed when the entity binds to the target site.

A compound or chemical complex designed or selected so as to bind to a target site may be further computationally optimised so that in its bound state it would preferably lack repulsive electrostatic interaction with the target protein. Such non-complementary (eg electrostatic) interactions include repulsive charge-charge, dipole-dipole and charge-dipole interactions. Specifically, the sum of all electrostatic interactions between the entity or other entity and the target site, when the entity is bound to the target site, preferably make a neutral or favourable contribution to the enthalpy of binding.

Specific computer software is available in the art to evaluate compound deformation energy and electrostatic interaction. Examples of programs designed for such uses include: Gaussian 92, revision C (Frisch, M J, Gaussian, Inc, Pittsburgh, Pa., USA); AMBER, version 4.0 (Kollman, Pa., University of California, San Francisco, Calif., USA); QUANTA/CHARMM; and Insight II/Discover (Accelrys, Inc, Burlington, Mass., USA). These programs may be implemented, for instance, using a Silicon Graphics O2 workstation or Intel CPU based Linux cluster. Other hardware systems and software packages will be known to the person skilled in the art.

Once a compound or chemical complex has been optimally designed or selected, as described above, modifications may be made to, for example, improve or modify its binding properties. Thus, for a compound, substitutions may be made in some of its atoms or side groups. Generally, initial substitutions of this kind will be conservative, that is the replacement group will have approximately the same size, shape, hydrophobicity and charge as the original group. It should, of course, be understood that components known in the art to alter conformation should be avoided. Such substituted chemical compounds may then be analysed for efficiency of fit to a specific target site by the same computer methods described in detail above.

Another approach is the computational screening of small molecule databases for compounds or chemical complexes that can interact in whole, or in part, to a target site. In this screening, the quality of fit of such entities to the target site may be judged either by shape complementarity or by estimated interaction energy (see, for example, Meng et al, 1992).

### SUMMARY OF THE INVENTION

In a first aspect, the present invention provides a method for identifying an agent for modulating the biological activity of an Fc receptor protein, said method comprising the steps of:
(i) generating a three-dimensional structure model of high responder FcγRIIa (HR_{S88}), low responder FcγRIIa (LR_{S88}) or a portion thereof, wherein said structure model comprises the three-dimensional structure of a target site with which an agent may interact and thereby modulate the biological activity of the receptor, and
(ii) identifying a candidate agent by designing or selecting a compound or chemical complex with a three-dimensional structure enabling interaction with said target site.

In a second aspect, the present invention provides a method for screening compounds and/or chemical complexes for a candidate agent for modulating the biological activity of an Fc receptor, said method comprising the steps of:
(i) generating a three-dimensional structure model of high responder FcγRIIa (HR_{S88}), low responder FcγRIIa (LR_{S88}) or a portion thereof, wherein said structure model comprises the three-dimensional structure of a target site with which an agent may interact and thereby modulate the biological activity of the receptor, and
(ii) screening said compounds and/or chemical complexes to identify any compound(s) or chemical complex(es) having a three-dimensional structure which enables interaction with said target site.

In a third aspect, the present invention provides a method for modifying a candidate agent for modulating the biological activity of an Fc receptor, said method comprising the steps of:
(i) generating a three-dimensional structure model of high responder FcγRIIa (HR_{S88}), low responder FcγRIIa (LR_{S88}) or a portion thereof, wherein said structure model comprises the three-dimensional structure of a target site with which an agent may interact and thereby modulate the biological activity of the receptor, and
(ii) modifying the candidate agent to provide an agent with a three-dimensional structure more favourable to providing the desired level of interaction with said target site than the candidate agent.

In a fourth aspect, the present invention provides a method of designing a variant of high responder FcγRIIa (HR_{S88}) or low responder FcγRIIa (LR_{S88}) with altered biological activity, said method comprising the steps of:
(i) generating a three-dimensional structure model of HR_{S88} or LR_{S88} or a portion thereof; and
(ii) modifying the model to provide a variant of HR_{S88} or LR_{S88} with altered biological activity.

In a fifth aspect, the present invention provides a computer for producing a three-dimensional structure model of high responder FcγRIIa (HR_{S88}), low responder FcγRIIa (LR_{S88}) or a portion thereof, said structure model comprising the three-dimensional structure of a target site to which an agent may interact and thereby modulate the activity of an Fc receptor, wherein said computer comprises:
(i) a machine-readable data storage medium (eg a magnetic or optical storage medium such as a hard drive, floppy disc or a CD-ROM) comprising the atomic coordinate data of Table 3;
(ii) a working memory for storing instructions for processing said atomic coordinate data contained on the machine-readable data storage medium;
(iii) a central processing unit coupled to said working memory and to said machine-readable data storage medium for processing said atomic coordinate data to generate said three-dimensional structure model; and
(iv) a display coupled to said central processing unit for displaying a representation of said three-dimensional structure model.

In a sixth aspect, the present invention provides a machine-readable data storage medium comprising the atomic coordinate data of Table 3.

In a seventh aspect, the present invention provides a candidate agent identified in accordance with the method of the first or second aspect, an agent produced in accordance with the third aspect or a variant of high responder FcγRIIa (HR_{S88}) or low responder FcγRIIa (LR_{S88}) designed in accordance with the fourth aspect.

In an eighth aspect, the present invention provides the use of the agent or a variant of high responder FcγRIIa (HR_{S88}) or low responder FcγRIIa (LR_{S88}) of the seventh aspect in the preparation of a medicament for modulating the biological activity of an Fc receptor in a subject.

In a ninth aspect, the present invention provides a method of modulating the biological activity of an Fc receptor in a subject, said method comprising administering a medicament comprising an agent or the variant of high responder FcγRIIa (HR_{S88}) or low responder FcγRIIa (LR_{S88}) of the seventh aspect.

In a tenth aspect, the present invention provides a method of producing a medicament, wherein said method comprises:
(i) identifying an agent in accordance with the method of the first aspect, identifying a compound(s) and/or chemical complex(es) in accordance with the method of the second aspect, or modifying a candidate agent in accordance with the method of the third aspect to provide a modified agent,
(ii) chemically synthesising said agent, compound(s) and/or chemical complex(es) or modified agent,
(iii) evaluating the ability of the synthesised agent, compound(s) and/or chemical complex(es) or modified agent to treat an Fc receptor-mediated disease or condition, and
(iv) formulating the synthesised agent, compound(s) and/or chemical complex(es) or modified agent with a suitable, pharmaceutically-acceptable delivery vehicle or adjuvant to produce said medicament.

In an eleventh aspect, the present invention provides a method of treating an Fc receptor-mediated disease or condition in a subject, said method comprising administering to said subject a pharmaceutically-effective amount of an agent or a variant of high responder FcγRIIa (HR_{S88}) or low responder FcγRIIa (LR_{S88}) which binds to a surface on an Fc receptor (FcR) selected from:
(a) the surface forming the immunoglobulin-binding site;
(b) the surface forming the dimerisation interface between two HR_{S88} or two LR_{S88} monomers of a dimerised receptor;
(c) the surface forming a large groove between two HR_{S88} or two LR_{S88} monomers of a dimerised receptor (site A); and
(d) the surface forming a cavity, channel and two identical pockets adjacent to the dimerisation interface between two HR_{S88} or two LR_{S88} monomers of a dimerised receptor (site B).

### BRIEF DESCRIPTION OF THE FIGURES

FIG. 1 provides photomicrographs of crystals of FcγRIIa expressed as the HR_{S88} wild-type (panel a) and LR_{F88} mutant (panel b) glycoproteins. (a) Crystals of the HR_{S88} wild-type FcγRIIa produced by the vapour diffusion method in a 2 μl sitting drop with the protein at (4 mg/ml in 75 mM NaCl, 5 mM Tris buffer pH 7.4). The crystallisation solution also contained 30% PEG 4000 and 0.2M ammonium sulfate. The crystals were formed at 18° C. (b) Crystals of the LR_{F88} mutant FcγRIIa produced by the vapor diffusion method in a 5 μl hanging drop with the protein solution at 8 mg/ml in 0.2 ammonium acetate, 0.1 sodium citrate buffer pH 5.6 and 30% PEG 4000. The crystals nucleated at 37° C. and were grown and maintained at 18° C.

FIG. 2 shows a comparison of crystal packing (lattice) contacts of the LR_{F88} mutant (panels a and b) and HR_{S88} wild-type (panels c and d) FcγRIIa glycoproteins. (a) High resolution crystal structure of the LR_{F88} mutant FcγRIIa showing electron density (F₀-F_{c} omit contoured at 3.0 σa) maps for a key lattice contact that involves the side chains of Phe88, Trp90 and Trp113 binding to a proline (P35*) guest ligand from a symmetry related protein monomer. Refined atomic coordinates that correspond to the electron density modules are shown as ball-and-stick representations. (b) Crystal packing of the ab-plane of LR_{F88} mutant of FcγRIIa with protein monomers displayed as carbon alpha traces. Locations of the key lattice contacts, illustrated in panel a, are indicated (*). (c) The crystal structure of the HR_{S88} wild-type FcγRIIa showing electron density (Fₒ-F_{c} omit contoured at 3.0 σ) maps showing that a solvent (water) molecule is bound in an similar location where P35* was bound in crystals of LR_{F88} mutant FcγRIIa. The wild-type S88 residue is clearly located in the electron density maps. (d) Crystal packing of the ab-plane of HR_{S88} wild-type of FcγRIIa with protein monomers displayed as carbon alpha traces. The crystal packing interactions are comprehensively different to those for the LR_{F88} mutant (see panel b).

FIG. 3 provides a diagrammatic representation of the structure of the predominant twofold dimer of HR_{S88} found in the crystalline state. (a) The HR_{S88} dimer in an orientation suitable for assembly in the membrane of a cell with the IgG binding surfaces of each protein monomer at the top and the terminal polypeptide residues facing toward the plasma membrane (bottom of page). (b) The HR_{S88} dimer has been rotated by 90° to generate this side-on view of the receptor assembly.

FIG. 4 provides a molecular model for the outside-to-inside signalling or activation complex of FcγRIIa. The model was generated by rigid body superposition of the coordinates for the HR_{S88} twofold dimer (FIG. 3) and those extracted for an FcγRIII-Fc complex (PDB code 1E4K) (Sondermann et al, 2000). The dimeric form of HR_{S88} found in the crystal lattice is shown by the modelled signalling complex to be capable of binding simultaneously to two Fc (or antibody) ligand molecules. The amino-(N) and carboxyl-(C) termini of the proteins are indicated. The antigen binding portions of the antibodies (Fab) emerge from the N-termini of the two bound Fc molecules.

FIG. 5 provides solvent-accessible surface views of the dimeric form of HR_{S88}. Three orthogonal views showing: (a) the receptor dimer in an orientation that clearly shows the large solvent-filled groove formed between the two receptor monomers; (b) a side-view of the receptor dimer, and; (c) an end-on view of the receptor dimer showing the surfaces on the two monomers that can interact with two antibody (IgG) ligands. The cavity and channel that resides below the groove is visible in the centre of the receptor dimer shown in panel (c). FIGS. 5 to 7 were prepared using the Insight II program package, version 98.0 (Accelrys), and Connolly solvent-accessible surfaces are depicted (Connolly, 1983).

FIG. 6 shows a cut-away solvent-accessible surface view showing one monomer of the HR_{S88} dimer. The locations of target sites for modulating agents are labelled and include a large solvent-filled groove (site A) and a cavity with an adjacent channel (site B). Locations of the deep pockets associated with site B are also marked (B′). Solvent accessible surfaces are shaded in grey. Regions that were inaccessible or buried to the solvent probe are shaded in black and represent the interface between monomers 1 and 2.

FIG. 7 provides a cut-away surface view of one receptor monomer with mapped locations for amino acid residues. The view is shown in the same orientation and is used in conjunction with FIG. 6. Amino acids primarily forming the target sites (A and B) for modulating agents are labelled in the one letter code.

FIG. 8 provides a schematic diagram showing the interactions that form the HR_{S88} dimer interface. Amino acid residues are followed by either (A) or (B) to indicate if the particular residue is derived from either receptor monomer 1 or 2. The key accompanying the diagram defines the nature of the interactions shown. The plot was generated with standard parameters using the LIGPLOT program (Wallace et al. 1995).

FIG. 9 provides schematic representations of the chemical structures of VIB153 and VIB197.

FIG. 10 shows predicted binding modes for VIB153 as docked into target sites A and B of the HR_{S88} crystallographic dimer. (a) VIB153 docked into site A. (b) VIB153 docked into site B. Cut-away solvent-accessible surface views (as described for FIGS. 6 and 7) with the predicted orientations of the ligand (stick representations) shown in the left panels. Schematic LIGPLOTS of the predicted interactions between the ligand and protein are shown in the right panels. Designations for monomer 1 (A), monomer 2 (B) of the receptor and the ligand (C) are shown after the residue number.

FIG. 11 shows predicted binding modes for VIB197 as docked into target sites A and B of the HR_{S88} crystallographic dimer. (a) VIB197 docked into site A. (b) VIB197 docked into site B. Cut-away solvent-accessible surface views (as described for FIGS. 6 and 7) with the predicted orientations of the ligand (stick representations) shown in the left panels. Schematic LIGPLOTS of the predicted interactions between the ligand and protein are shown in the right panels.

### DETAILED DESCRIPTION OF THE INVENTION

The present applicants have determined the crystal structure of HR_{S88} wild-type of FcγRIIa which crystallised in C222₁ and have found that there are significant differences between the crystal packing observed for this receptor and that previously observed for LR_{F88}. In addition, the present applicants have elucidated from the crystal structure a novel dimeric form of the FcγRIIa receptor, one which readily accommodates two Fc portions of human immunoglobulin (eg IgG1). It is considered that this novel dimeric form is intrinsically involved in the signalling complex of FcγRIIa and, therefore, is of use in elucidating the biology and modulation of this receptor and other cell membrane-associated protein receptors. More particularly, the novel dimeric form of HR_{S88} identified by the present applicants is of use in identifying and modifying agents for modulating the biological activity of Fc receptors.

Thus, in a first aspect, the present invention provides a method for identifying an agent for modulating the biological activity of an Fc receptor protein (FcR), said method comprising the steps of:
(i) generating a three-dimensional structure model of high responder FcγRIIa (HR_{S88}), low responder FcγRIIa (LR_{S88}) or a portion thereof, wherein said structure model comprises the three-dimensional structure of a target site with which an agent may interact and thereby modulate the biological activity of the receptor, and
(ii) identifying a candidate agent by designing or selecting a compound or chemical complex with a three-dimensional structure enabling interaction with said target site.

In a preferred form, the method is for identifying a candidate agent for modulation of the interaction between the monomers of a dimer of HR_{S88} or LR_{S88}, said method comprising the steps of:
(i) generating a three-dimensional structure model of a dimer of HR_{S88} or LR_{S88} or a portion thereof in which portions of each monomer are represented, wherein said structure model comprises the three-dimensional structure of a target site with which an agent may interact and thereby modulate the interaction between the monomers; and
(ii) identifying a candidate agent by designing or selecting a compound or chemical complex with a three-dimensional structure enabling interaction with said target site.

In a second aspect, the present invention provides a method for screening compounds and/or chemical complexes for a candidate agent for modulating the biological activity of an Fc receptor protein (FcR), said method comprising the steps of:
(i) generating a three-dimensional structure model of high responder FcγRIIa (HR_{S88}), low responder FcγRIIa (LR_{S88}) or a portion thereof, wherein said structure model comprises the three-dimensional structure of a target site with which an agent may interact and thereby modulate the biological activity of the receptor, and
(ii) screening said compounds and/or chemical complexes to identify any compound(s) or chemical complex(es) having a three-dimensional structure which enables interaction with said target site.

In a preferred form, there is provided a method for screening compounds and/or chemical complexes for a candidate agent for modulation of the interaction between the monomers of a dimer of HR_{S88} or LR_{S88,} said method comprising the steps of:
(i) generating a three-dimensional structure model of a dimer of HR_{S88} or LR_{S88} or a portion thereof in which portions of each monomer are represented, wherein said structure model comprises the three-dimensional structure of a target site with which an agent may interact and thereby modulate the interaction between the monomers; and
(ii) screening said compounds and/or chemical complexes to identify any compound(s) or chemical complex(es) having a three-dimensional structure which enables interaction with said target site.

In a third aspect, the present invention provides a method for modifying a candidate agent for modulating the biological activity of an Fc receptor protein (FcR), said method comprising the steps of:
(i) generating a three-dimensional structure model of high responder FcγRIIa (HR_{S88}), low responder FcγRIIa (LR_{S88}) or a portion thereof, wherein said structure model comprises the three-dimensional structure of a target site with which an agent may interact and thereby modulate the biological activity of the receptor, and
(ii) modifying the candidate agent to provide an agent with a three-dimensional structure more favourable to providing a desired level of interaction with said target site than the candidate agent.

In a preferred form, there is provided a method for modifying a candidate agent for modulation of the interaction between the monomers of a dimer of HR_{S88} or LR_{S88} to provide an agent with improved activity, said method comprising the steps of:
(i) generating a three-dimensional structure model of a dimer of HR_{S88} or LR_{S88} or a portion thereof in which portions of each monomer are represented, wherein said structure model comprises the three-dimensional structure of a target site with which an agent may interact and thereby modulate the interaction between the monomers; and
(ii) modifying the candidate agent to provide an agent with a three-dimensional structure more favourable to providing a desired level of interaction with said target site than the candidate agent.

The methods of the first to third aspects of the invention are preferably in silico methods.

The three-dimensional structure model generated in step (i) of each of the methods of the first to third aspects comprise, at least, the three-dimensional structure of a target site to which a candidate agent or a developed agent (ie modified candidate agent) may interact (eg bind) with, preferably HR_{S88} or a portion thereof or, otherwise, a dimer of HR_{S88} or a portion thereof. The atomic coordinate data for the amino acids within the three-dimensional structure model of HR_{S88} is provided in Table 3 hereinafter. Thus, the three-dimensional structure model generated in the methods of the first to third aspects is preferably generated using at least the atomic coordinate data of Table 3.

The atomic coordinate data of Table 3 represents one of the monomers of the dimer of HR_{S88}. As would be understood by the person skilled in the art, the other monomer of the dimer can be readily generated by applying the symmetry operations of space group C222₁ to the atomic coordinates of Table 3. The appropriate symmetry operation is:

$$\begin{matrix}1 & 0 & 0 \\ 0 & - 1 & 0 \\ 0 & 0 & - 1\end{matrix} \times \begin{matrix}X \\ Y \\ Z\end{matrix} + \begin{matrix}0 \\ 155.88 \\ - 88.10\end{matrix} = \begin{matrix}XSYM \\ YSYM \\ ZSYM\end{matrix}$$

In the method of the first aspect, the step of identifying a candidate agent (ie step (ii)) may be achieved by methods described above for designing and selecting compounds or chemical complexes with three-dimensional structures that fit and interact with a target site.

The method of the first aspect may further comprise a step of assessing the deformation of energy of the candidate agent when brought from the free state to the target site-interacting state (eg bound state). Preferably, the deformation of energy is not greater than 10 kcal/mole and, more preferably, not greater than 7 kcal/mole. Additionally or alternatively to the step of assessing the deformation of the candidate agent, the method of the first aspect may comprise a step of assessing the enthalpy of the interaction (eg binding) of the candidate agent with the target site. Preferably, the candidate agent shall make a neutral or favourable contribution to the enthalpy of the interaction.

In the method of the second aspect, the step of screening compounds and/or chemical complexes to identify any compound(s) or chemical complex(es) with a three-dimensional structure enabling interaction with the target site (ie step (ii)) may be achieved by methods described above. The screened compounds and/or chemical complexes may belong to a library or database of suitable compounds and/or chemical complexes (eg ACD-SC (Available Chemicals Directory Screening Compounds), MDL Inc, San Leandro, Calif., USA).

In the method of the third aspect, the step of modifying a candidate agent (ie step (ii)) may be achieved by methods described above such as substituting one or more groups (eg functional groups) on compounds.

In the methods of the first to third aspects, the candidate agent and agent is preferably selected from small chemical entities (SCE) and monoclonal antibodies.

In the methods of the first to third aspects, the agents may modulate biological activity by, for example, binding to or mimicking the action of an FcR, disrupting cellular signal transduction through an FcR by, for example, preventing dimerisation of two FcR proteins, or enhancing cellular signal transduction or binding to an FcR by, for example, enhancing dimerisation of two FcR proteins.

In the methods of the first to third aspects, the target site is preferably a surface on the HR_{S88} or LR_{S88} selected from:
(a) the surface forming the immunoglobulin-binding site;
(b) the surface forming the dimerisation interface between two HR_{S88} or LR_{S88} monomers of a dimerised receptor;
(c) the surface forming a large groove between two HR_{S88} or LR_{S88} monomers of a dimerised receptor (hereinafter referred to as site A); and
(d) the surface forming a cavity, channel and two identical pockets adjacent to the dimerisation interface between two HR_{S88} or LR_{S88} monomers of a dimerised receptor (hereinafter referred to as site B).

As used herein, the term “interface” refers to the group of atoms and residues from separate polypeptide chains (eg monomer 1 and monomer 2 of FcγRIIa) that are in direct contact (ie hydrophobic, van der Waals or electrostatic contact) and nearby residues, not necessarily in direct contact, which may be reasonably regarded as contributing to the protein:protein interaction.

Where the target site is the immunoglobulin-binding site, preferably the surface comprises a structure defined by the conformation of amino acid residues 113-116, 129, 131, 133, 134, 155, 156 and 158-160.

Where the target site is the dimerisation interface, preferably the surface comprises a structure defined by the conformation of amino acid residues 26, 33, 54-56, 58, 102, 103, 105, 142 and 143 of one monomer of the HR_{S88} dimer (or LR_{S88} dimer) and the equivalent residues of the other monomer of the dimer.

Where the target site is site A of an HR_{S88} dimer, preferably the surface comprises a structure defined by the conformation of amino acid residues 22-24, 60, 107, 109, 110, 112, 114-118, 131, 133-138, 140 and 160 of one monomer of the HR_{S88} dimer (or the LR_{S88} dimer) and the equivalent residues of the other monomer of the dimer.

Where the target site is site B of an HR_{S88} dimer (or LR_{S88} dimer), preferably the surface comprises a structure defined by the conformation of amino acid residues 12-16, 26, 96, 100 and 105 of one monomer of the HR_{S88} dimer (or LR_{S88} dimer) and the equivalent residues of the other monomer of the dimer.

Agents which interact (eg bind) to one of the preferred target sites (a) to (d) above, may modulate the biological activity of an FcR protein, particularly FcγRIIa, by inhibiting or enhancing cellular signal transduction by the receptor or through inhibiting or enhancing binding of the receptor to the Fc portion of an immunoglobulin protein (eg IgG) or fragment thereof.

In a fourth aspect, the present invention provides a method of designing a variant of high responder FcγRIIa (HR_{S88}) or low responder FcγRIIa (LR_{S88}) with altered biological activity, said method comprising the steps of:
(i) generating a three-dimensional structure model of HR_{S88} or LR_{S88} or a portion thereof; and
(ii) modifying the model to provide a variant of HR_{S88} or LR_{S88} with altered biological activity.

By the term “variant”, we refer to any to a molecule that differs from HR_{S88} or LR_{S88} but which retains similarity in biological activity. A variant may therefore have substantial overall structural similarity with HR_{S88} or LR_{S88} or only structural similarity with one or more regions of HR_{S88} or LR_{S88} (eg a soluble HR_{S88} variant may only have structural similarity to the extracellular region of HR_{S88}). Typically, a variant of HR_{S88} or LR_{S88} will be provided by, or be the result of, the addition of one or more amino acids to the amino acid sequence of HR_{S88} or LR_{S88}, deletion of one or more amino acids from the amino acid sequence of HR_{S88} or LR_{S88} and/or substitution of one or more amino acids of the amino acid sequence of HR_{S88} or LR_{S88}. Inversion of amino acids and other mutational changes that result in the alteration of the amino acid sequence are also encompassed. The substitution of an amino acid may involve a conservative or non-conservative amino acid substitution. By conservative amino acid substitution, it is meant that an amino acid residue is replaced with another amino acid having similar characteristics and which does not substantially alter the biological function of the polypeptide. Exemplary conservative amino acid substitutions are provided in Table A below. Particular conservative substitutions envisaged are: G, A, V, I, L, M; D, E, N, Q; S, C, T; K, R, H: and P, Nα-alkylamino acids. In general, conservative amino acid substitutions will be selected on the basis that they do not have any substantial effect on (a) the structure of the peptide backbone in the region of the substitution, (b) the charge or hydrophobicity of the polypeptide at the site of substitution, and/or (c) the bulk of the side chain at the site of substitution. Where a variant is prepared by synthesis, the variant may also include an amino acid or amino acids not encoded by the genetic code, such as γ-carboxyglutamic acid and hydroxyproline. For example, D-amino acids rather than L-amino acids may be included. In one preferred embodiment of a variant according to the fourth aspect, the variant is a mimetic of HR_{S88} such as a peptido-mimetic.

In a preferred form of the method of the fourth aspect, there is provided a method of designing a variant of a dimer of HR_{S88} or LR_{S88} with altered biological activity, said method comprising the steps of:
(i) generating a three-dimensional structure model of a dimer of HR_{S88} or LR_{S88} or a portion thereof in which portions of each monomer are represented; and
(ii) modifying the model to provide a variant of the dimer of HR_{S88} or LR_{S88} with altered biological activity.

The method of the fourth aspect of the invention is preferably an in silico method.

TABLE A
Exemplary conservative amino acid substitutions

| | Conservative Substitutions |
|---|---|
| Ala | Val*, Leu, Ile |
| Arg | Lys*, Gln, Asn |
| Asn | Gln*, His, Lys, Arg, Asp |
| Asp | Glu*, Asn |
| Cys | Ser |
| Gln | Asn*, His, Lys, |
| Glu | Asp*, γ-carboxyglutamic acid (Gla) |
| Gly | Pro |
| His | Asn, Gln, Lys, Arg* |
| Ile | Leu*, Val, Met, Ala, Phe, norleucine (Nle) |
| Leu | Nle, Ile*, Val, Met, Ala, Phe |
| Lys | Arg*, Gln, Asn, ornithine (Orn) |
| Met | Leu*, Ile, Phe, Nle |
| Phe | Leu*, Val, Ile, Ala |
| Pro | Gly*, hydroxyproline (Hyp), Ser, Thr |
| Ser | Thr |
| Thr | Ser |
| Trp | Tyr |
| Tyr | Trp, Phe*, Thr, Ser |
| Val | Ile, Leu*, Met, Phe, Ala, Nle |

*indicates preferred conservative substitutions

The method of the fourth aspect provides a means for designing proteins that have altered beneficial functions by analysing the structure and interactions between individual amino acids of the protein. For example, therapeutic proteins having improved binding to Ig or immune complexes of Ig can be designed to be used as therapeutic compounds to prevent immune complex binding to cells or enhance biological responses such as cellular signal transduction upon binding of FcR to Ig or complexes thereof. Thus, recombinant soluble FcR engineered to contain improvements can be produced on the basis of the knowledge of the three-dimensional structure.

The three-dimensional structure model generated in step (i) of the method of the fourth aspect comprises, at least, the three-dimensional structure of a target site to which a candidate agent or a developed agent may interact (eg bind) with, preferably, HR_{S88} or dimer thereof. Preferably, the three-dimensional structure model generated in step (i) of the method of the fourth aspect is generated using at least the atomic coordinate data of Table 3.

A recombinant protein according to a variant of HR_{S88}, or a dimer thereof (or LR_{S88} or dimer thereof), may be prepared by any of the methods well known to the person skilled in the art. For example, where the modifications made to provide the variant involve one or more amino acid substitution, deletion and/or insertion, the recombinant protein may be prepared by firstly generating a DNA molecule encoding the variant protein by site-directed mutagenesis of a DNA molecule encoding the Fc receptor (eg HR_{S88}), and thereafter expressing the DNA molecule in a suitable host cell. A DNA molecule encoding FcγRIIa, and methods for expressing DNA molecules encoding FcγRIIa and variants thereof (including soluble variants), are disclosed in International patent application no PCT/AU87/00159 (Publication no WO 87/07277) and International patent application no PCT/AU95/00606 (Publication no WO 96/08512). The disclosures of these two International patent applications are to be regarded as incorporated herein by reference.

In the methods of the first to fourth aspects, the model may further comprise an Fc portion of a protein which binds to HR_{S88} or an immunoglobulin (eg IgG) or portion thereof. In a preferred form, the atomic coordinates for the Fc portion/immunoglobulin of the model are obtained from the coordinates for an FcγRIII-Fc complex provided in the Protein Data Bank (see PDB code 1E4K).

In a fifth aspect, the present invention provides a computer for producing a three-dimensional structure model of high responder FcγRIIa (HR_{S88}), low responder FcγRIIa (LR_{S88})or a portion thereof, said structure model comprising the three-dimensional structure of a target site to which an agent may interact and thereby modulate the activity of an Fc receptor protein (FcR), wherein said computer comprises:
(i) a machine-readable data storage medium (eg a magnetic or optical storage medium such as a hard drive, floppy disc or a CD-ROM) comprising the atomic coordinate data of Table 3;
(ii) a working memory for storing instructions for processing said atomic coordinate data contained on the machine-readable data storage medium;
(iii) a central processing unit coupled to said working memory and to said machine-readable data storage medium for processing said atomic coordinate data to generate said three-dimensional structure model; and
(iv) a display coupled to said central processing unit for displaying a representation of said three-dimensional structure model.

The computer may further comprise:
(v) means for receiving and storing atomic coordinate data for a range of chemical components and substituents, wherein the central processing unit is capable of interacting with said receiving and storing means and selects from said range of chemical components and substituents suitable chemical components and substituents to assemble a compound or chemical complex which, based upon a three-dimensional structure generated by said central processing unit, a representation of which may be provided on said display simultaneously with the representation of said three-dimensional structural model of HR_{S88}, LR_{S88} or a portion thereof, is capable of interaction with said target site; and/or
(vi) means for receiving and storing atomic coordinate data for a range of compounds and/or chemical complexes, wherein the central processing unit is capable of interacting with said receiving and storing means to generate a three-dimensional structure for a compound or chemical complex selected from the range of compounds and/or chemical complexes, provide a representation of said three-dimensional structure on said display simultaneously with the representation of said three-dimensional structural model of HR_{S88}, LR_{S88} or a portion thereof, and thereby enable an assessment of whether said selected compound or chemical complex is capable of interaction with said target site.

The atomic coordinate data for the range of chemical components and substituents and the atomic coordinate data for the range of compounds and/or chemical complexes, can be obtained from suitable databases.

In a sixth aspect, the present invention provides a machine-readable data storage medium comprising the atomic coordinate data of Table 3.

In a seventh aspect, the present invention provides a candidate agent identified in accordance with the method of the first or second aspect, an agent produced in accordance with the third aspect or a variant of HR_{S88} designed in accordance with the fourth aspect.

The candidate agent, agent or variant of the seventh aspect may be used to prepare a medicament to modulate the biological activity of FcR (in particular, an FcR selected from FcαR, FcεR, FcγR such as FcγRIIa, FcγRIIb and FcγRIIc, and mixtures thereof) in a subject. The medicament can be used for, for example, reducing IgG-mediated tissue damage; stimulating an IgG humoral immune response in an animal; and improving the therapeutic effects of an antibody that is administered to an animal to treat, by opsonisation or FcγR-dependent effector functions (eg antibody-dependent FcγR-mediated cytotoxicity, phagocytosis or release of cellular mediators), a particular disease, including, but not limited to, inflammatory diseases, autoimmune diseases, cancer or infectious disease (eg oral infections such as HIV, herpes, bacterial infections, yeast infections or parasite infections).

Preferably, the agent of the seventh aspect is selected from small chemical entities (SCE) and monoclonal antibodies.

Thus, in an eighth aspect, the present invention provides the use of the candidate agent, agent or variant of the seventh aspect in the preparation of a medicament for modulating the biological activity of FcR (particularly, FcγRIIa) in a subject.

And, in a ninth aspect, the present invention provides a method of modulating the biological activity of FcR (particularly, FcγRIIa) in a subject, said method comprising administering a medicament comprising a candidate agent, agent or variant of the seventh aspect.

The subject referred to in the eighth and ninth aspects may be a human or other animal (eg companion animals and livestock).

In producing the medicament of the present invention, the candidate agent, agent or variant of the seventh aspect may be formulated with any pharmaceutically-acceptable delivery vehicle or adjuvant for administration to the subject. Administration may be by any suitable mode including, for example, intramuscular injection, intravenous administration, nasal administration via an aerosol spray, and oral administration.

The amount of the candidate agent, agent or variant of the seventh aspect that may be administered to a subject may vary upon a number of factors including the immune status of the subject and the severity of any disease or condition being treated. However, by way of example, an agent according to the seventh aspect may be administered to a subject at a dose of about 0.001 to 10 mg/kg body weight, preferably from 0.1 to 1 mg/kg body weight.

In a tenth aspect, the present invention provides a method of producing a medicament, wherein said method comprises:
(i) identifying an agent in accordance with the method of the first aspect, identifying a compound(s) and/or chemical complex(es) in accordance with the method of the second aspect, or modifying a candidate agent in accordance with the method of the third aspect to provide a modified agent,
(ii) chemically synthesising said agent, compound(s) and/or chemical complex(es) or modified agent,
(iii) evaluating the ability of the synthesised agent, compound(s) and/or chemical complex(es) or modified agent to treat an Fc receptor-mediated disease or condition, and
(iv) formulating the synthesised agent, compound(s) and/or chemical complex(es) or modified agent with a suitable, pharmaceutically-acceptable delivery vehicle or adjuvant to produce said medicament.

In an eleventh aspect, the present invention provides a method of treating an Fc receptor-mediated disease or condition in a subject, said method comprising administering to said subject a pharmaceutically-effective amount of an agent or a variant of HR_{S88} or LR_{S88} which binds to a surface on an Fc receptor (FcR) selected from:
(a) the surface forming the immunoglobulin-binding site;
(b) the surface forming the dimerisation interface between two HR_{S88} or LR_{S88} monomers of a dimerised receptor;
(c) the surface forming a large groove between two HR_{S88} or LR_{S88} monomers of a dimerised receptor (site A); and
(d) the surface forming a cavity, channel and two identical pockets adjacent to the dimerisation interface between two HR_{S88} or LR_{S88} monomers of a dimerised receptor (site B).

Preferably, the agent or a variant of HR_{S88} or LR_{S88}, in binding to one of said surfaces on FcR, causes inhibition of binding of immunoglobulin to FcR.

Preferably, the FcR referred to in the eleventh aspect, is selected from the group consisting of FcαR, FcεR, FcγR (eg FcγRIIa, FcγRIIb and FcγRIIc) and mixtures thereof. Most preferably, the said FcR is FcγRIIa.

The FcR-mediated disease or condition which may be treated by the method of the eleventh aspect may be selected from the group consisting of; IgG-mediated tissue damage, IgE-mediated diseases or conditions, inflammation, an autoimmune disease (eg rheumatoid arthritis, systemic lupus erythematosus, immune thrombocytopenia, neutropenia, and hemolytic anaemias).

The method of the eleventh aspect may also be used to treat an FcR-mediated disease or condition wherein aggregates of antibodies are produced or where immune complexes are produced by contact of antibody with intrinsic or extrinsic antigen. Such diseases include immune complex diseases, autoimmune diseases, infectious diseases (eg Dengue virus-dengue hemorrhagic fever and measles virus infection) and vasculitities (eg polyarteritis nodosa, and systemic vasculitis).

In order that the nature of the present invention may be more clearly understood, preferred forms thereof will now be described with reference to the following non-limiting examples, in which:

Table 1 provides a summary of statistics for the X-ray data and crystallographic refinements used for structure determination of the HR_{S88} glycoprotein. Data from the HR_{S88} crystal were obtained using a MicroMax007/R-Axis IV⁺⁺ rotating anode X-ray generator system operated at 40 kV and 20 mA. Data were reduced and scaled using the DENZO and Scalepack programs from the HKL suite version 1.97 (HKL Research Inc, USA). The crystal structure was solved and refined using the CNS program package version 1.0 (Brunger et al, 1998);

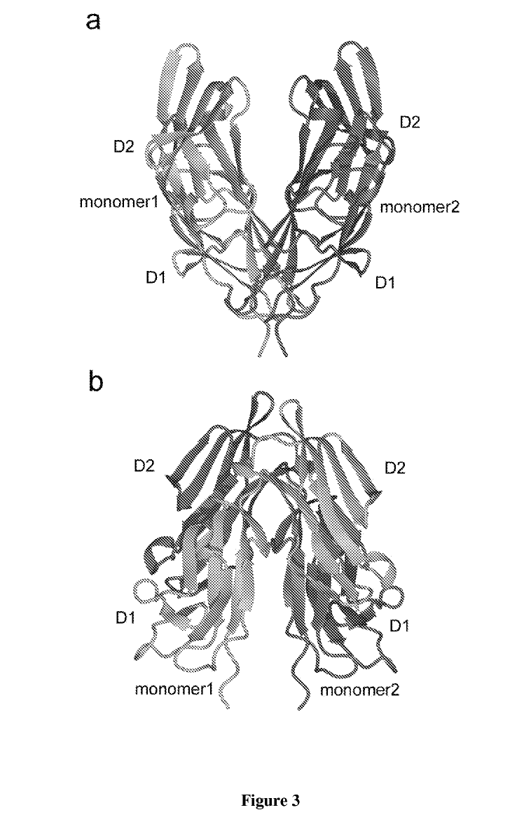

Table 2 provides the interatomic distances less than 4 Å relating the protein monomers forming the predominant crystallographic dimer of HR_{S88} wild-type FcγRIIa crystals. The dimeric receptor form from which these distances were calculated is easily generated using standard symmetry operators associated with the provided atomic coordinates (Table 3). The dimeric receptor form is illustrated in FIG. 3; and

Table 3 provides the refined atomic coordinates for the crystal structure of HR_{S88}.

Table 4 provides the atomic coordinates for the highest ranked docked orientation of the VIB 153 ligand into site A of the crystal structure of the HR_{S88} dimer.

Table 5 provides the atomic coordinates for the highest ranked docked orientation of the VIB 153 ligand into site B of the crystal structure of the HR_{S88} dimer.

Table 6 provides the atomic coordinates for the highest ranked docked orientation of the VIB 197 ligand into site A of the crystal structure of the HR_{S88} dimer.

Table 7 provides the atomic coordinates for the highest ranked docked orientation of the VIB 197 ligand into site B of HR_{S88} dimer.

### EXAMPLES

### Example 1

### Determination of the 2.3 Å Crystal Structure of the Wild-Type HR_{S88} FcγRIIa Glycoprotein

Materials and Methods

Description of Protein Preparation

Wild-type HR_{S88} FcγRIIa cDNA (Arg at position 134 and Ser at position 88) was produced by splice overlap extension PCR and expressed in SF21 insect cells using the baculovirus expression system. Briefly, SF21 cells in Gibco SF900 media (Invitrogen Australia Pty Ltd, Vic, Australia) were grown to a density of 2×10⁶ cells/ml in 10×200 ml flasks. Cells were infected by the addition of 5 ml virus stock/flask and maintained at 27° C. for 72 h. The receptor was purified supernatant by anion exchange over Q-sepharose, followed by an affinity chromatography step over heat aggregated immunoglobulin coupled sepharose, as previously described for LR_{F88} FcγRIIa (Powell et al, 1999). Purified HR_{S88} glycoprotein was dialysed into 75 mM NaCl, 5 mM Tris buffer pH 7.4 and concentrated to between 5 and 10 mg/ml using a Micosep 10K concentrator (Pall Corporation, NY, USA) and maintained at 4° C. until crystallisation experiments.

Crystals of the HR_{S88} glycoprotein were produced by the vapour diffusion method in a 2 μl sitting drop with the protein at 4 mg/ml in 75 mM NaCl, 5 mM Tris buffer pH 7.4. The crystallisation solution also contained 30% PEG 4000 and 0.2M ammonium sulfate. The crystals were formed at 18° C. A crystal was removed from the solution and subjected to X-ray diffraction analysis. Data from the HR_{S88} crystal were obtained using a MicroMax007/R-Axis IV⁺⁺ rotating anode X-ray generator system operated at 40 kV and 20 mA. Data were reduced and scaled using the DENZO and Scalepack programs from the HKL suite version 1.97 (HKL Research Inc, USA). The crystal structure was solved and refined using the CNS program package version 1.0 (Brunger et al, 1998).

Results and Discussion

Crystallographic data and refinement statistics are summarised in Table 1, while the refined atomic coordinates for the crystal structure are found in Table 3.

The structure of a crystal of the LR_{F88} mutant had been previously described (Protein Data Bank code 1FCG: Maxwell et al, 1999, and Powell et al,. 1999). LR_{F88} formed when, during cloning and amplification of the original cDNA used for expression and crystallisation of the human LR allele of FcγRIIa, a single amino acid substitution was introduced (replacing a serine for a phenylalanine at position 88 in the nucleotide sequence) by the non-proofreading Taq polymerase used for the polymerase chain reaction. The LR_{F88} glycoprotein was over-expressed in insect cells and the crystal structure determined at 2.0 A. However, it was not obvious what the effect of the F88 mutation on the crystal structure was since the LR_{F88} monomer shares a very similar overall three-dimensional structure to all structures for related Fc receptors that have been determined and deposited in the Protein Data Bank (PDB).

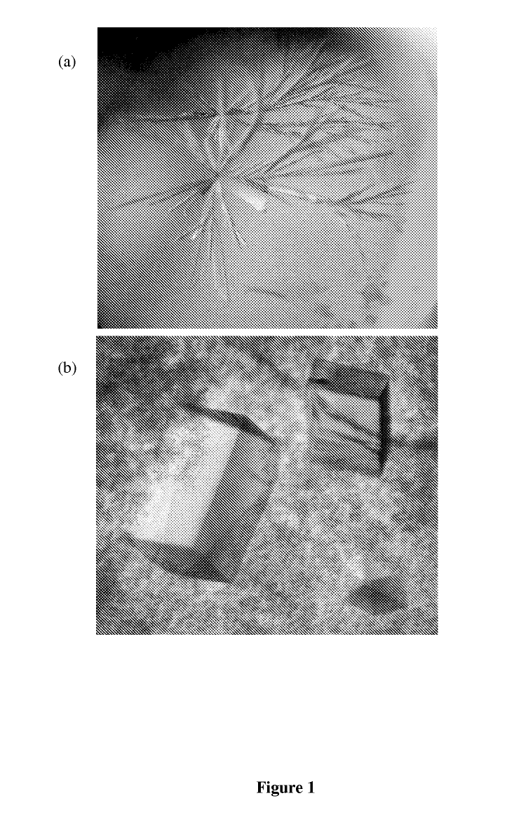

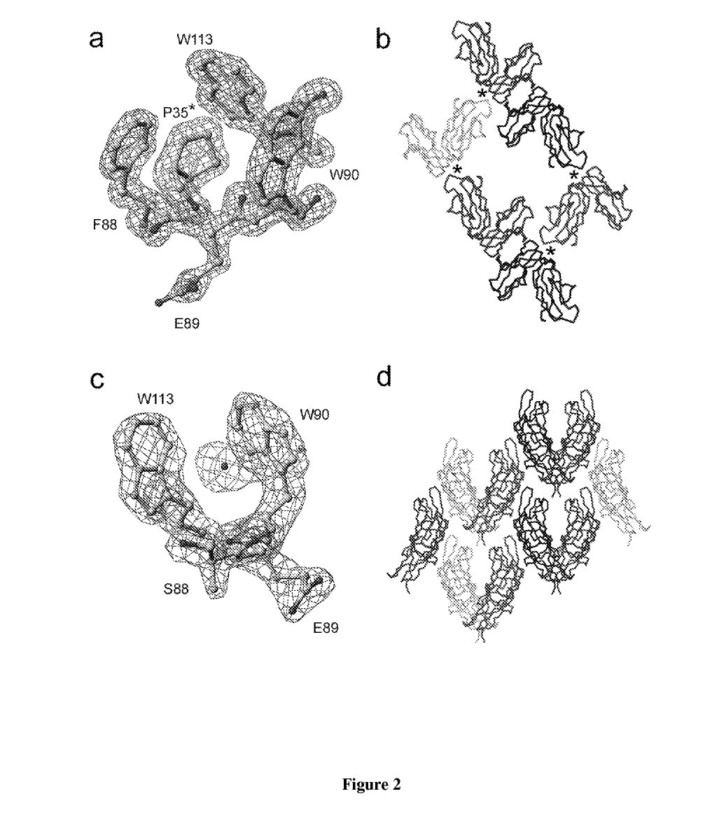

As can be seen in FIG. 1, crystals of the wild-type HR_{S88} glycoprotein form as bundles of needles with numerous branching points and growth defects while the LR_{F88} mutant are almost always single well-formed crystals. The different growth properties provided an indication that the lattice of LR_{F88} crystals was substantially more uniform by its capacity to grow reliably in three-dimensions. Moreover, as can be seen from FIG. 2, the arrangement of molecules in the HR_{S88} crystal lattice is different from that previously determined for the structure of LR_{F88}. That is, the molecules of the HR_{S88} crystal are arranged in dimers that could plausibly be present in cell membranes, the molecules in the LR_{F88} FcγRIIa crystal are not so arranged.

### Example 2

### Description of Crystallographic Dimers of the Wild-Type HR_{S88} FcγRIIa Glycoprotein and Modelling the Outside-To-Inside Cell Signalling Assembly

Methods and Materials

Description and uses of the Crystallographic Dimers of HR_{S88} FcγRIIa.

Using information associated with its C222₁ space group, the crystalline lattice of HR_{S88} (Table 1) was constructed with the provided atomic coordinates (Table 3). The following criteria were applied to identify possible cell signalling assemblies of FcγRIIa as they occur in the membrane of cells: (1) the interactions between crystallographic dimers should be numerous and chemically compatible; (2) the residues that normally are anchored in the cell membrane by a tethering polypeptide would emerge in positions that would allow the dimer to associate in the context of the membrane; and (3) the active (IgG binding) portions of the receptor should be located in a position to bind two ligands. The symmetry transformation matrix used to generate the other half (monomer 2) of the dimer from the provided atomic coordinates (Table 3) was:

$$\begin{matrix}1 & 0 & 0 \\ 0 & - 1 & 0 \\ 0 & 0 & - 1\end{matrix} \times \begin{matrix}X \\ Y \\ Z\end{matrix} + \begin{matrix}0 \\ 155.88 \\ - 88.10\end{matrix} = \begin{matrix}XSYM \\ YSYM \\ ZSYM\end{matrix}$$

Results and Discussion

Using the listed criteria (1) to (3), an FcγRIIa dimer was identified that was related by a crystallographic two-fold (ie a 180° rotation around a central axis) to form within the HR_{S88} crystals (FIG. 3). It is recognised that the residues forming the interface between the two monomers represent preferred targets for agents that modulate the biological activity of Fc receptors, and particularly FcγRIIa.

A detailed listing of the atomic contacts (with a 4 Å cutoff applied) that constitute the HR_{S88} FcγRIIa dimer interface is provided in Table 2. Modulating agents can be targeted to the interface residues by exploiting these residues and all FcγRIIa residues within a 10 Å radius of any listed interface residue. Examples of such modulating agents include small chemical entities (SCE), monoclonal antibodies, and modified soluble versions of the or other interacting molecules.

It is considered that wild type low responder FcγRIIa (LR_{S88}) would form the same crystal lattice as HR_{S88} and, consequently, would generate substantially the same three-dimensional crystal structure as HR_{S88}. In particular, it is considered that the model for the dimeric form of HR_{S88} represents a valid model for LR_{S88.} That is, since the HR_{S88} and LR_{S88} polymorphic variants of FcγRIIa differ in amino acid sequence only at position 134 (Arg versus His), located well away from the monomer 1:monomer 2 interface, it is considered that an identical or substantially similar dimer interface exists for the wild type LR_{S88} form of the receptor.

### Example 3

### Modelling the Outside-To-Inside Cell Signalling Assembly of FcγRIIa

Materials and Methods

The transformed atomic coordinates for the crystallographic twofold dimer of HR_{S88} and the deposited coordinates for the complex of a FcγRIII in complex with the Fc portion of a human Fc (PDB code 1E4K) (Sondermann et al. 2000) were used to model the outside-to-in signalling assembly of FcγRIIa. The structurally conserved residues from domain 2 of the FcγRIIa and FcγRIII receptor coordinates were used for the rigid body superposition using least squares fitting.

Results and Discussion

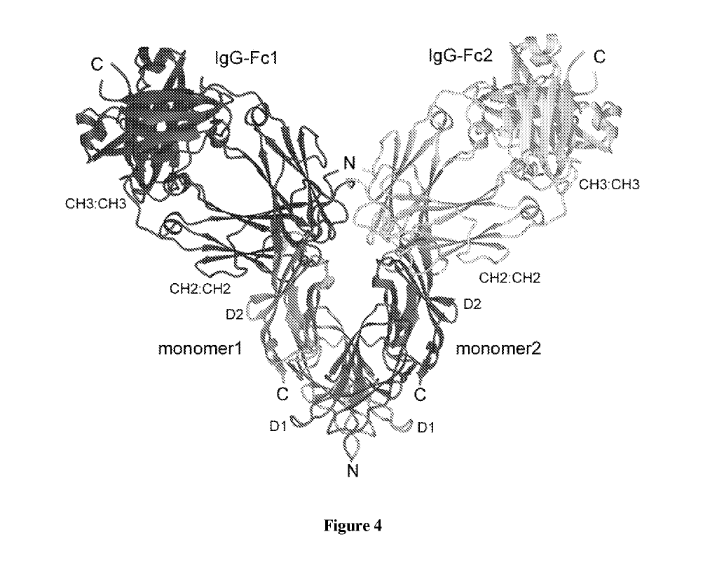

A model of the outside-to-in signalling assembly of FcγRIIa was generated and is shown in FIG. 4. Docking of the complex to the dimer of HR_{S88} demonstrated that two molecules of the Fc ligand can bind (without steric clashes) to the ordered assembly (FIG. 4). Antigen binding portions (Fab) of an intact IgG are also compatible with this model of the signalling assembly as they emerge on the same side of the modelled complex.

TABLE 1
Summary of X-ray data collection and refinement statistics

| FcγRIIa | HR_{S88} |
|---|---|
| Space group | C222₁ |
| Cell dimensions (Å) | a = 50.04 Å |
| | b = 77.94 Å |
| | c = 88.10 Å |
| Resolution range, overall (last shell) (Å) | 30-2.3 (2.38-2.3) |
| Completeness, overall (last shell) (%) | 97.2 (96.0) |
| R_{sym}, overall (last shell) | 0.10 (0.27) |
| I/σ(I), overall (last shell) | 13.2 (4.9) |
| R_{work} (R_{free}), entire resolution range | 0.206 (0.275) |

TABLE 2
List of interatomic distances less than 4.00 Å between
protein monomers in the crystallographic dimer of the
HR_{S88} FcγRIIa glycoprotein

| Monomer 1 | | Monomer 2 | Atom | |
|---|---|---|---|---|
| residue | Atom name (i) | residue | name (j) | i − j distances (Å) |
| 11LYS | NZ | 97HIS | NE2 | 3.86 |
| 26THR | CG2 | 105THR | CB | 3.81 |
| 26THR | CG2 | 105THR | OG1 | 3.57 |
| 28THR | CG2 | 104GLU | OE1 | 3.96 |
| 33ARG | CZ | 102GLU | O | 3.86 |
| 33ARG | NH2 | 102GLU | N | 3.91 |
| 33ARG | NH2 | 102GLU | C | 3.79 |
| 33ARG | NH2 | 102GLU | O | 2.72 |
| 54GLN | CB | 103GLY | O | 3.63 |
| 54GLN | CB | 143GLN | CG | 3.82 |
| 54GLN | CG | 143GLN | CG | 3.92 |
| 54GLN | OE1 | 103GLY | CA | 3.36 |
| 55PRO | N | 103GLY | O | 3.89 |
| 55PRO | CD | 102GLU | O | 3.37 |
| 55PRO | CD | 103GLY | CA | 3.73 |
| 55PRO | CD | 103GLY | C | 3.70 |
| 55PRO | CD | 103GLY | O | 3.76 |
| 55PRO | CG | 102GLU | O | 3.21 |
| 56SER | N | 103GLY | O | 3.39 |
| 56SER | CB | 103GLY | O | 3.75 |
| 56SER | CB | 104GLU | CA | 3.96 |
| 56SER | CB | 104GLU | C | 3.99 |
| 56SER | CB | 105THR | N | 3.42 |
| 56SER | CB | 105THR | CB | 3.86 |
| 56SER | CB | 105THR | OG1 | 3.18 |
| 56SER | OG | 104GLU | CA | 3.36 |
| 56SER | OG | 104GLU | CG | 3.61 |
| 56SER | OG | 104GLU | C | 3.48 |
| 56SER | OG | 105THR | N | 2.78 |
| 56SER | OG | 105THR | CA | 3.76 |
| 56SER | OG | 105THR | CB | 3.63 |
| 56SER | OG | 105THR | OG1 | 3.50 |
| 56SER | C | 143GLN | NE2 | 3.79 |
| 56SER | O | 143GLN | CG | 3.99 |
| 56SER | O | 143GLN | CD | 3.93 |
| 56SER | O | 143GLN | NE2 | 3.00 |
| 58ARG | CD | 142PRO | CB | 3.84 |
| 58ARG | CD | 142PRO | CG | 3.88 |
| 58ARG | NE | 142PRO | CG | 3.99 |
| 58ARG | NH2 | 140SER | CB | 3.96 |
| 97HIS | NE2 | 11LYS | NZ | 3.86 |
| 102GLU | N | 33ARG | NH2 | 3.91 |
| 102GLU | c | 33ARG | NH2 | 3.79 |
| 102GLU | O | 33ARG | CZ | 3.86 |
| 102GLU | O | 33ARG | NH2 | 2.72 |
| 102GLU | O | 55PRO | CD | 3.37 |
| 102GLU | O | 55PRO | CG | 3.21 |
| 103GLY | CA | 54GLN | OE1 | 3.36 |
| 103GLY | CA | 55PRO | CD | 3.73 |
| 103GLY | C | 55PRO | CD | 3.70 |
| 103GLY | O | 54GLN | CB | 3.63 |
| 103GLY | O | 55PRO | N | 3.89 |
| 103GLY | O | 55PRO | CD | 3.76 |
| 103GLY | O | 56SER | N | 3.39 |
| 103GLY | O | 56SER | CB | 3.75 |
| 104GLU | CA | 56SER | CB | 3.96 |
| 104GLU | CA | 56SER | OG | 3.36 |
| 104GLU | CG | 56SER | OG | 3.61 |
| 104GLU | OE1 | 28THR | CG2 | 3.96 |
| 104GLU | C | 56SER | CB | 3.99 |
| 104GLU | C | 56SER | OG | 3.48 |
| 105THR | N | 56SER | CB | 3.42 |
| 105THR | N | 56SER | OG | 2.78 |
| 105THR | CA | 56SER | OG | 3.76 |
| 105THR | CB | 26THR | CG2 | 3.81 |
| 105THR | CB | 56SER | CB | 3.86 |
| 105THR | CB | 56SER | OG | 3.63 |
| 105THR | OG1 | 26THR | CG2 | 3.57 |
| 105THR | OG1 | 56SER | CB | 3.18 |
| 105THR | OG1 | 56SER | OG | 3.50 |
| 140SER | CB | 58ARG | NH2 | 3.96 |
| 142PRO | CB | 58ARG | CD | 3.84 |
| 142PRO | CG | 58ARG | CD | 3.88 |
| 142PRO | CG | 58ARG | NE | 3.99 |
| 143GLN | CG | 54GLN | CB | 3.82 |
| 143GLN | CG | 54GLN | CG | 3.92 |
| 143GLN | CG | 56SER | O | 3.99 |
| 143GLN | CD | 56SER | O | 3.93 |
| 143GLN | NE2 | 56SER | C | 3.79 |
| 143GLN | NE2 | 56SER | O | 3.00 |

TABLE 3
Atomic coordinates of the HR_{S88} wild-type FcγRIIa glycoprotein
determined at 2.3 Å resolution

| | | | | | | | | | | | |
|---|---|---|---|---|---|---|---|---|---|---|---|
| ATOM | 1 | CB | ALA | A | 4 | 37.328 | 74.566 | −50.900 | 1.00 | 25.51 | A |
| ATOM | 2 | C | ALA | A | 4 | 36.177 | 76.774 | −50.697 | 1.00 | 27.86 | A |
| ATOM | 3 | O | ALA | A | 4 | 35.671 | 77.172 | −49.645 | 1.00 | 28.39 | A |
| ATOM | 4 | N | ALA | A | 4 | 38.266 | 76.334 | −49.433 | 1.00 | 27.64 | A |
| ATOM | 5 | CA | ALA | A | 4 | 37.527 | 76.070 | −50.703 | 1.00 | 29.11 | A |
| ATOM | 6 | N | PRO | A | 5 | 35.571 | 76.933 | −51.881 | 1.00 | 28.14 | A |
| ATOM | 7 | CD | PRO | A | 5 | 35.993 | 76.443 | −53.206 | 1.00 | 27.08 | A |
| ATOM | 8 | CA | PRO | A | 5 | 34.272 | 77.599 | −51.959 | 1.00 | 27.50 | A |
| ATOM | 9 | CB | PRO | A | 5 | 33.852 | 77.368 | −53.409 | 1.00 | 29.43 | A |
| ATOM | 10 | CG | PRO | A | 5 | 35.162 | 77.288 | −54.137 | 1.00 | 28.45 | A |
| ATOM | 11 | C | PRO | A | 5 | 33.283 | 76.969 | −50.991 | 1.00 | 27.61 | A |
| ATOM | 12 | O | PRO | A | 5 | 33.251 | 75.750 | −50.833 | 1.00 | 27.85 | A |
| ATOM | 13 | N | PRO | A | 6 | 32.481 | 77.794 | −50.308 | 1.00 | 26.71 | A |
| ATOM | 14 | CD | PRO | A | 6 | 32.468 | 79.265 | −50.257 | 1.00 | 24.97 | A |
| ATOM | 15 | CA | PRO | A | 6 | 31.504 | 77.224 | −49.379 | 1.00 | 25.30 | A |
| ATOM | 16 | CB | PRO | A | 6 | 30.846 | 78.458 | −48.766 | 1.00 | 26.43 | A |
| ATOM | 17 | CG | PRO | A | 6 | 31.899 | 79.520 | −48.888 | 1.00 | 26.62 | A |
| ATOM | 18 | C | PRO | A | 6 | 30.525 | 76.442 | −50.258 | 1.00 | 26.17 | A |
| ATOM | 19 | O | PRO | A | 6 | 30.410 | 76.718 | −51.452 | 1.00 | 24.57 | A |
| ATOM | 20 | N | LYS | A | 7 | 29.824 | 75.470 | −49.694 | 1.00 | 25.75 | A |
| ATOM | 21 | CA | LYS | A | 7 | 28.884 | 74.716 | −50.503 | 1.00 | 23.95 | A |
| ATOM | 22 | CB | LYS | A | 7 | 28.771 | 73.284 | −49.984 | 1.00 | 26.06 | A |
| ATOM | 23 | CG | LYS | A | 7 | 28.404 | 73.171 | −48.528 | 1.00 | 30.44 | A |
| ATOM | 24 | CD | LYS | A | 7 | 29.115 | 71.983 | −47.881 | 1.00 | 33.77 | A |
| ATOM | 25 | CE | LYS | A | 7 | 28.844 | 70.676 | −48.605 | 1.00 | 32.83 | A |
| ATOM | 26 | NZ | LYS | A | 7 | 29.564 | 69.546 | −47.950 | 1.00 | 31.06 | A |
| ATOM | 27 | C | LYS | A | 7 | 27.522 | 75.398 | −50.517 | 1.00 | 22.70 | A |
| ATOM | 28 | O | LYS | A | 7 | 27.071 | 75.951 | −49.508 | 1.00 | 25.59 | A |
| ATOM | 29 | N | ALA | A | 8 | 26.882 | 75.380 | −51.678 | 1.00 | 19.26 | A |
| ATOM | 30 | CA | ALA | A | 8 | 25.572 | 75.986 | −51.837 | 1.00 | 16.47 | A |
| ATOM | 31 | CB | ALA | A | 8 | 25.081 | 75.793 | −53.272 | 1.00 | 14.83 | A |
| ATOM | 32 | C | ALA | A | 8 | 24.599 | 75.333 | −50.857 | 1.00 | 14.90 | A |
| ATOM | 33 | O | ALA | A | 8 | 24.810 | 74.201 | −50.422 | 1.00 | 12.60 | A |
| ATOM | 34 | N | VAL | A | 9 | 23.541 | 76.053 | −50.503 | 1.00 | 12.93 | A |
| ATOM | 35 | CA | VAL | A | 9 | 22.539 | 75.515 | −49.596 | 1.00 | 14.61 | A |
| ATOM | 36 | CB | VAL | A | 9 | 22.347 | 76.417 | −48.354 | 1.00 | 14.78 | A |
| ATOM | 37 | CG1 | VAL | A | 9 | 21.267 | 75.839 | −47.465 | 1.00 | 11.37 | A |
| ATOM | 38 | CG2 | VAL | A | 9 | 23.659 | 76.529 | −47.580 | 1.00 | 11.06 | A |
| ATOM | 39 | C | VAL | A | 9 | 21.216 | 75.391 | −50.340 | 1.00 | 16.97 | A |
| ATOM | 40 | O | VAL | A | 9 | 20.711 | 76.371 | −50.902 | 1.00 | 14.30 | A |
| ATOM | 41 | N | LEU | A | 10 | 20.667 | 74.180 | −50.352 | 1.00 | 18.76 | A |
| ATOM | 42 | CA | LEU | A | 10 | 19.408 | 73.921 | −51.038 | 1.00 | 21.43 | A |
| ATOM | 43 | CB | LEU | A | 10 | 19.429 | 72.529 | −51.669 | 1.00 | 19.76 | A |
| ATOM | 44 | CG | LEU | A | 10 | 18.519 | 72.306 | −52.877 | 1.00 | 21.34 | A |
| ATOM | 45 | CD1 | LEU | A | 10 | 18.699 | 70.883 | −53.370 | 1.00 | 22.80 | A |
| ATOM | 46 | CD2 | LEU | A | 10 | 17.064 | 72.561 | −52.507 | 1.00 | 26.42 | A |
| ATOM | 47 | C | LEU | A | 10 | 18.242 | 74.046 | −50.060 | 1.00 | 24.97 | A |
| ATOM | 48 | O | LEU | A | 10 | 18.105 | 73.259 | −49.113 | 1.00 | 24.44 | A |
| ATOM | 49 | N | LYS | A | 11 | 17.409 | 75.051 | −50.307 | 1.00 | 27.78 | A |
| ATOM | 50 | CA | LYS | A | 11 | 16.251 | 75.351 | −49.476 | 1.00 | 27.26 | A |
| ATOM | 51 | CB | LYS | A | 11 | 16.116 | 76.871 | −49.342 | 1.00 | 31.83 | A |
| ATOM | 52 | CG | LYS | A | 11 | 15.169 | 77.361 | −48.256 | 1.00 | 41.29 | A |
| ATOM | 53 | CD | LYS | A | 11 | 15.150 | 78.894 | −48.217 | 1.00 | 44.24 | A |
| ATOM | 54 | CE | LYS | A | 11 | 14.359 | 79.432 | −47.032 | 1.00 | 46.33 | A |
| ATOM | 55 | NZ | LYS | A | 11 | 14.305 | 80.928 | −47.011 | 1.00 | 48.07 | A |
| ATOM | 56 | C | LYS | A | 11 | 14.989 | 74.766 | −50.109 | 1.00 | 25.90 | A |
| ATOM | 57 | O | LYS | A | 11 | 14.828 | 74.772 | −51.329 | 1.00 | 24.01 | A |
| ATOM | 58 | N | LEU | A | 12 | 14.101 | 74.258 | −49.267 | 1.00 | 22.40 | A |
| ATOM | 59 | CA | LEU | A | 12 | 12.854 | 73.674 | −49.720 | 1.00 | 20.82 | A |
| ATOM | 60 | CB | LEU | A | 12 | 12.754 | 72.241 | −49.180 | 1.00 | 23.82 | A |
| ATOM | 61 | CG | LEU | A | 12 | 11.650 | 71.274 | −49.605 | 1.00 | 25.37 | A |
| ATOM | 62 | CD1 | LEU | A | 12 | 11.528 | 71.199 | −51.110 | 1.00 | 28.91 | A |
| ATOM | 63 | CD2 | LEU | A | 12 | 11.981 | 69.910 | −49.043 | 1.00 | 25.79 | A |
| ATOM | 64 | C | LEU | A | 12 | 11.713 | 74.550 | −49.198 | 1.00 | 22.31 | A |
| ATOM | 65 | O | LEU | A | 12 | 11.632 | 74.834 | −48.000 | 1.00 | 18.88 | A |
| ATOM | 66 | N | GLU | A | 13 | 10.848 | 75.003 | −50.100 | 1.00 | 22.80 | A |
| ATOM | 67 | CA | GLU | A | 13 | 9.723 | 75.844 | −49.702 | 1.00 | 23.45 | A |
| ATOM | 68 | CB | GLU | A | 13 | 10.039 | 77.317 | −49.988 | 1.00 | 24.49 | A |
| ATOM | 69 | CG | GLU | A | 13 | 11.245 | 77.829 | −49.211 | 1.00 | 30.67 | A |
| ATOM | 70 | CD | GLU | A | 13 | 11.052 | 77.778 | −47.692 | 1.00 | 37.34 | A |
| ATOM | 71 | OE1 | GLU | A | 13 | 12.061 | 77.923 | −46.964 | 1.00 | 38.67 | A |
| ATOM | 72 | OE2 | GLU | A | 13 | 9.901 | 77.605 | −47.221 | 1.00 | 35.70 | A |
| ATOM | 73 | C | GLU | A | 13 | 8.401 | 75.451 | −50.364 | 1.00 | 21.64 | A |
| ATOM | 74 | O | GLU | A | 13 | 8.280 | 75.439 | −51.586 | 1.00 | 21.11 | A |
| ATOM | 75 | N | PRO | A | 14 | 7.404 | 75.070 | −49.552 | 1.00 | 22.25 | A |
| ATOM | 76 | CD | PRO | A | 14 | 6.054 | 74.682 | −49.994 | 1.00 | 19.93 | A |
| ATOM | 77 | CA | PRO | A | 14 | 7.520 | 75.003 | −48.090 | 1.00 | 20.14 | A |
| ATOM | 78 | CB | PRO | A | 14 | 6.098 | 74.633 | −47.651 | 1.00 | 18.12 | A |
| ATOM | 79 | CG | PRO | A | 14 | 5.560 | 73.868 | −48.828 | 1.00 | 19.88 | A |
| ATOM | 80 | C | PRO | A | 14 | 8.590 | 73.963 | −47.701 | 1.00 | 17.42 | A |
| ATOM | 81 | O | PRO | A | 14 | 9.025 | 73.166 | −48.533 | 1.00 | 16.00 | A |
| ATOM | 82 | N | PRO | A | 15 | 9.011 | 73.946 | −46.429 | 1.00 | 15.29 | A |
| ATOM | 83 | CD | PRO | A | 15 | 8.481 | 74.749 | −45.311 | 1.00 | 14.30 | A |
| ATOM | 84 | CA | PRO | A | 15 | 10.041 | 73.006 | −45.967 | 1.00 | 12.10 | A |
| ATOM | 85 | CB | PRO | A | 15 | 10.473 | 73.615 | −44.638 | 1.00 | 9.66 | A |
| ATOM | 86 | CG | PRO | A | 15 | 9.177 | 74.131 | −44.095 | 1.00 | 15.49 | A |
| ATOM | 87 | C | PRO | A | 15 | 9.681 | 71.520 | −45.846 | 1.00 | 13.16 | A |
| ATOM | 88 | O | PRO | A | 15 | 10.543 | 70.696 | −45.550 | 1.00 | 9.50 | A |
| ATOM | 89 | N | TRP | A | 16 | 8.423 | 71.172 | −46.088 | 1.00 | 15.42 | A |
| ATOM | 90 | CA | TRP | A | 16 | 7.990 | 69.782 | −45.986 | 1.00 | 16.15 | A |
| ATOM | 91 | CB | TRP | A | 16 | 6.509 | 69.689 | −46.351 | 1.00 | 16.61 | A |
| ATOM | 92 | CG | TRP | A | 16 | 5.675 | 70.758 | −45.680 | 1.00 | 17.66 | A |
| ATOM | 93 | CD2 | TRP | A | 16 | 5.564 | 71.001 | −44.273 | 1.00 | 13.83 | A |
| ATOM | 94 | CE2 | TRP | A | 16 | 4.686 | 72.097 | −44.105 | 1.00 | 13.21 | A |
| ATOM | 95 | CE3 | TRP | A | 16 | 6.120 | 70.398 | −43.136 | 1.00 | 14.38 | A |
| ATOM | 96 | CD1 | TRP | A | 16 | 4.882 | 71.692 | −46.294 | 1.00 | 14.02 | A |
| ATOM | 97 | NE1 | TRP | A | 16 | 4.286 | 72.499 | −45.353 | 1.00 | 13.81 | A |
| ATOM | 98 | CZ2 | TRP | A | 16 | 4.350 | 72.603 | −42.844 | 1.00 | 10.19 | A |
| ATOM | 99 | CZ3 | TRP | A | 16 | 5.785 | 70.902 | −41.879 | 1.00 | 14.24 | A |
| ATOM | 100 | CH2 | TRP | A | 16 | 4.905 | 71.996 | −41.747 | 1.00 | 8.98 | A |
| ATOM | 101 | C | TRP | A | 16 | 8.810 | 68.847 | −46.883 | 1.00 | 15.83 | A |
| ATOM | 102 | O | TRP | A | 16 | 8.915 | 69.065 | −48.087 | 1.00 | 18.78 | A |
| ATOM | 103 | N | ILE | A | 17 | 9.393 | 67.805 | −46.301 | 1.00 | 14.39 | A |
| ATOM | 104 | CA | ILE | A | 17 | 10.179 | 66.869 | −47.094 | 1.00 | 14.04 | A |
| ATOM | 105 | CB | ILE | A | 17 | 11.363 | 66.283 | −46.296 | 1.00 | 15.18 | A |
| ATOM | 106 | CG2 | ILE | A | 17 | 12.203 | 67.417 | −45.717 | 1.00 | 15.64 | A |
| ATOM | 107 | CG1 | ILE | A | 17 | 10.859 | 65.356 | −45.190 | 1.00 | 15.69 | A |
| ATOM | 108 | CD1 | ILE | A | 17 | 11.988 | 64.693 | −44.427 | 1.00 | 10.23 | A |
| ATOM | 109 | C | ILE | A | 17 | 9.331 | 65.729 | −47.643 | 1.00 | 12.16 | A |
| ATOM | 110 | O | ILE | A | 17 | 9.824 | 64.881 | −48.385 | 1.00 | 14.46 | A |
| ATOM | 111 | N | ASN | A | 18 | 8.056 | 65.700 | −47.275 | 1.00 | 11.03 | A |
| ATOM | 112 | CA | ASN | A | 18 | 7.154 | 64.677 | −47.789 | 1.00 | 14.19 | A |
| ATOM | 113 | CB | ASN | A | 18 | 6.934 | 63.534 | −46.769 | 1.00 | 16.50 | A |
| ATOM | 114 | CG | ASN | A | 18 | 6.568 | 64.025 | −45.374 | 1.00 | 19.11 | A |
| ATOM | 115 | OD1 | ASN | A | 18 | 6.958 | 65.119 | −44.956 | 1.00 | 19.77 | A |
| ATOM | 116 | ND2 | ASN | A | 18 | 5.838 | 63.192 | −44.633 | 1.00 | 18.62 | A |
| ATOM | 117 | C | ASN | A | 18 | 5.853 | 65.352 | −48.182 | 1.00 | 15.93 | A |
| ATOM | 118 | O | ASN | A | 18 | 5.177 | 65.976 | −47.355 | 1.00 | 13.86 | A |
| ATOM | 119 | N | VAL | A | 19 | 5.529 | 65.245 | −49.469 | 1.00 | 16.61 | A |
| ATOM | 120 | CA | VAL | A | 19 | 4.344 | 65.875 | −50.029 | 1.00 | 13.78 | A |
| ATOM | 121 | CB | VAL | A | 19 | 4.742 | 67.002 | −51.000 | 1.00 | 15.49 | A |
| ATOM | 122 | CG1 | VAL | A | 19 | 5.586 | 68.050 | −50.279 | 1.00 | 9.76 | A |
| ATOM | 123 | CG2 | VAL | A | 19 | 5.507 | 66.423 | −52.168 | 1.00 | 14.50 | A |
| ATOM | 124 | C | VAL | A | 19 | 3.421 | 64.923 | −50.775 | 1.00 | 15.61 | A |
| ATOM | 125 | O | VAL | A | 19 | 3.702 | 63.733 | −50.927 | 1.00 | 10.94 | A |
| ATOM | 126 | N | LEU | A | 20 | 2.307 | 65.471 | −51.244 | 1.00 | 17.56 | A |
| ATOM | 127 | CA | LEU | A | 20 | 1.330 | 64.689 | −51.982 | 1.00 | 18.29 | A |
| ATOM | 128 | CB | LEU | A | 20 | −0.065 | 64.926 | −51.405 | 1.00 | 16.98 | A |
| ATOM | 129 | CG | LEU | A | 20 | −0.205 | 64.535 | −49.932 | 1.00 | 22.12 | A |
| ATOM | 130 | CD1 | LEU | A | 20 | −1.545 | 65.006 | −49.403 | 1.00 | 23.03 | A |
| ATOM | 131 | CD2 | LEU | A | 20 | −0.057 | 63.026 | −49.779 | 1.00 | 18.43 | A |
| ATOM | 132 | C | LEU | A | 20 | 1.356 | 65.058 | −53.458 | 1.00 | 17.98 | A |
| ATOM | 133 | O | LEU | A | 20 | 1.887 | 66.097 | −53.844 | 1.00 | 14.60 | A |
| ATOM | 134 | N | GLN | A | 21 | 0.782 | 64.184 | −54.276 | 1.00 | 22.50 | A |
| ATOM | 135 | CA | GLN | A | 21 | 0.710 | 64.382 | −55.714 | 1.00 | 24.05 | A |
| ATOM | 136 | CB | GLN | A | 21 | −0.140 | 63.276 | −56.346 | 1.00 | 25.20 | A |
| ATOM | 137 | CG | GLN | A | 21 | 0.330 | 62.821 | −57.719 | 1.00 | 34.51 | A |
| ATOM | 138 | CD | GLN | A | 21 | 1.401 | 61.743 | −57.658 | 1.00 | 38.66 | A |
| ATOM | 139 | OE1 | GLN | A | 21 | 2.025 | 61.413 | −58.673 | 1.00 | 40.24 | A |
| ATOM | 140 | NE2 | GLN | A | 21 | 1.613 | 61.179 | −56.469 | 1.00 | 36.99 | A |
| ATOM | 141 | C | GLN | A | 21 | 0.081 | 65.742 | −56.003 | 1.00 | 24.43 | A |
| ATOM | 142 | O | GLN | A | 21 | −0.958 | 66.086 | −55.440 | 1.00 | 23.60 | A |
| ATOM | 143 | N | GLU | A | 22 | 0.735 | 66.513 | −56.868 | 1.00 | 25.07 | A |
| ATOM | 144 | CA | GLU | A | 22 | 0.274 | 67.840 | −57.275 | 1.00 | 25.59 | A |
| ATOM | 145 | CB | GLU | A | 22 | −1.238 | 67.835 | −57.541 | 1.00 | 32.18 | A |
| ATOM | 146 | CG | GLU | A | 22 | −1.700 | 66.719 | −58.466 | 1.00 | 38.99 | A |
| ATOM | 147 | CD | GLU | A | 22 | −0.601 | 66.252 | −59.407 | 1.00 | 45.17 | A |
| ATOM | 148 | OE1 | GLU | A | 22 | −0.095 | 67.077 | −60.201 | 1.00 | 47.98 | A |
| ATOM | 149 | OE2 | GLU | A | 22 | −0.239 | 65.055 | −59.343 | 1.00 | 46.41 | A |
| ATOM | 150 | C | GLU | A | 22 | 0.617 | 68.968 | −56.308 | 1.00 | 23.76 | A |
| ATOM | 151 | O | GLU | A | 22 | 0.324 | 70.129 | −56.588 | 1.00 | 23.03 | A |
| ATOM | 152 | N | ASP | A | 23 | 1.212 | 68.642 | −55.166 | 1.00 | 21.08 | A |
| ATOM | 153 | CA | ASP | A | 23 | 1.607 | 69.686 | −54.229 | 1.00 | 18.77 | A |
| ATOM | 154 | CB | ASP | A | 23 | 2.343 | 69.105 | −53.015 | 1.00 | 18.42 | A |
| ATOM | 155 | CG | ASP | A | 23 | 1.409 | 68.695 | −51.900 | 1.00 | 15.03 | A |
| ATOM | 156 | OD1 | ASP | A | 23 | 0.199 | 68.965 | −52.010 | 1.00 | 20.54 | A |
| ATOM | 157 | OD2 | ASP | A | 23 | 1.889 | 68.110 | −50.908 | 1.00 | 15.31 | A |
| ATOM | 158 | C | ASP | A | 23 | 2.579 | 70.587 | −54.982 | 1.00 | 19.17 | A |
| ATOM | 159 | O | ASP | A | 23 | 3.324 | 70.123 | −55.848 | 1.00 | 19.65 | A |
| ATOM | 160 | N | SER | A | 24 | 2.567 | 71.872 | −54.658 | 1.00 | 16.27 | A |
| ATOM | 161 | CA | SER | A | 24 | 3.473 | 72.809 | −55.294 | 1.00 | 16.64 | A |
| ATOM | 162 | CB | SER | A | 24 | 2.897 | 74.228 | −55.228 | 1.00 | 14.99 | A |
| ATOM | 163 | OG | SER | A | 24 | 3.674 | 75.124 | −56.003 | 1.00 | 22.16 | A |
| ATOM | 164 | C | SER | A | 24 | 4.785 | 72.725 | −54.518 | 1.00 | 16.42 | A |
| ATOM | 165 | O | SER | A | 24 | 4.796 | 72.826 | −53.289 | 1.00 | 16.93 | A |
| ATOM | 166 | N | VAL | A | 25 | 5.886 | 72.521 | −55.232 | 1.00 | 16.60 | A |
| ATOM | 167 | CA | VAL | A | 25 | 7.186 | 72.414 | −54.590 | 1.00 | 15.07 | A |
| ATOM | 168 | CB | VAL | A | 25 | 7.768 | 70.999 | −54.754 | 1.00 | 16.05 | A |
| ATOM | 169 | CG1 | VAL | A | 25 | 9.199 | 70.947 | −54.186 | 1.00 | 14.10 | A |
| ATOM | 170 | CG2 | VAL | A | 25 | 6.868 | 69.990 | −54.049 | 1.00 | 13.03 | A |
| ATOM | 171 | C | VAL | A | 25 | 8.185 | 73.409 | −55.152 | 1.00 | 14.67 | A |
| ATOM | 172 | O | VAL | A | 25 | 8.322 | 73.546 | −56.364 | 1.00 | 19.72 | A |
| ATOM | 173 | N | THR | A | 26 | 8.888 | 74.098 | −54.264 | 1.00 | 14.69 | A |
| ATOM | 174 | CA | THR | A | 26 | 9.880 | 75.073 | −54.688 | 1.00 | 13.85 | A |
| ATOM | 175 | CB | THR | A | 26 | 9.443 | 76.505 | −54.328 | 1.00 | 16.46 | A |
| ATOM | 176 | OG1 | THR | A | 26 | 8.163 | 76.779 | −54.915 | 1.00 | 17.62 | A |
| ATOM | 177 | CG2 | THR | A | 26 | 10.476 | 77.515 | −54.827 | 1.00 | 12.37 | A |
| ATOM | 178 | C | THR | A | 26 | 11.235 | 74.806 | −54.043 | 1.00 | 15.63 | A |
| ATOM | 179 | O | THR | A | 26 | 11.353 | 74.764 | −52.816 | 1.00 | 13.98 | A |
| ATOM | 180 | N | LEU | A | 27 | 12.247 | 74.619 | −54.887 | 1.00 | 15.51 | A |
| ATOM | 181 | CA | LEU | A | 27 | 13.616 | 74.376 | −54.440 | 1.00 | 13.60 | A |
| ATOM | 182 | CB | LEU | A | 27 | 14.214 | 73.171 | −55.178 | 1.00 | 15.22 | A |
| ATOM | 183 | CG | LEU | A | 27 | 13.571 | 71.798 | −54.939 | 1.00 | 18.97 | A |
| ATOM | 184 | CD1 | LEU | A | 27 | 14.057 | 70.812 | −55.984 | 1.00 | 17.41 | A |
| ATOM | 185 | CD2 | LEU | A | 27 | 13.912 | 71.304 | −53.536 | 1.00 | 21.22 | A |
| ATOM | 186 | C | LEU | A | 27 | 14.451 | 75.618 | −54.741 | 1.00 | 14.36 | A |
| ATOM | 187 | O | LEU | A | 27 | 14.417 | 76.146 | −55.853 | 1.00 | 13.17 | A |
| ATOM | 188 | N | THR | A | 28 | 15.195 | 76.091 | −53.752 | 1.00 | 13.92 | A |
| ATOM | 189 | CA | THR | A | 28 | 16.021 | 77.265 | −53.961 | 1.00 | 16.70 | A |
| ATOM | 190 | CB | THR | A | 28 | 15.541 | 78.442 | −53.090 | 1.00 | 15.56 | A |
| ATOM | 191 | OG1 | THR | A | 28 | 14.171 | 78.727 | −53.405 | 1.00 | 15.26 | A |
| ATOM | 192 | CG2 | THR | A | 28 | 16.384 | 79.697 | −53.369 | 1.00 | 9.94 | A |
| ATOM | 193 | C | THR | A | 28 | 17.485 | 76.963 | −53.663 | 1.00 | 20.35 | A |
| ATOM | 194 | O | THR | A | 28 | 17.814 | 76.320 | −52.657 | 1.00 | 23.49 | A |
| ATOM | 195 | N | CYS | A | 29 | 18.361 | 77.416 | −54.554 | 1.00 | 18.99 | A |
| ATOM | 196 | CA | CYS | A | 29 | 19.789 | 77.201 | −54.394 | 1.00 | 19.55 | A |
| ATOM | 197 | C | CYS | A | 29 | 20.443 | 78.488 | −53.943 | 1.00 | 19.87 | A |
| ATOM | 198 | O | CYS | A | 29 | 20.554 | 79.429 | −54.715 | 1.00 | 24.81 | A |
| ATOM | 199 | CB | CYS | A | 29 | 20.413 | 76.764 | −55.714 | 1.00 | 18.28 | A |
| ATOM | 200 | SG | CYS | A | 29 | 22.064 | 76.035 | −55.502 | 1.00 | 17.99 | A |
| ATOM | 201 | N | GLN | A | 30 | 20.883 | 78.529 | −52.694 | 1.00 | 24.09 | A |
| ATOM | 202 | CA | GLN | A | 30 | 21.529 | 79.725 | −52.160 | 1.00 | 25.73 | A |
| ATOM | 203 | CB | GLN | A | 30 | 21.099 | 79.943 | −50.708 | 1.00 | 23.71 | A |
| ATOM | 204 | CG | GLN | A | 30 | 19.591 | 80.049 | −50.535 | 1.00 | 28.89 | A |
| ATOM | 205 | CD | GLN | A | 30 | 19.175 | 80.116 | −49.080 | 1.00 | 29.93 | A |
| ATOM | 206 | OE1 | GLN | A | 30 | 19.571 | 79.273 | −48.276 | 1.00 | 26.88 | A |
| ATOM | 207 | NE2 | GLN | A | 30 | 18.368 | 81.116 | −48.735 | 1.00 | 30.72 | A |
| ATOM | 208 | C | GLN | A | 30 | 23.048 | 79.599 | −52.244 | 1.00 | 24.93 | A |
| ATOM | 209 | O | GLN | A | 30 | 23.624 | 78.604 | −51.803 | 1.00 | 23.68 | A |
| ATOM | 210 | N | GLY | A | 31 | 23.690 | 80.613 | −52.814 | 1.00 | 26.58 | A |
| ATOM | 211 | CA | GLY | A | 31 | 25.134 | 80.592 | −52.947 | 1.00 | 26.89 | A |
| ATOM | 212 | C | GLY | A | 31 | 25.642 | 81.650 | −53.905 | 1.00 | 28.08 | A |
| ATOM | 213 | O | GLY | A | 31 | 25.017 | 81.922 | −54.933 | 1.00 | 27.70 | A |
| ATOM | 214 | N | ALA | A | 32 | 26.779 | 82.248 | −53.561 | 1.00 | 28.29 | A |
| ATOM | 215 | CA | ALA | A | 32 | 27.401 | 83.286 | −54.375 | 1.00 | 27.17 | A |
| ATOM | 216 | CB | ALA | A | 32 | 28.759 | 83.663 | −53.788 | 1.00 | 21.01 | A |
| ATOM | 217 | C | ALA | A | 32 | 27.572 | 82.849 | −55.827 | 1.00 | 28.55 | A |
| ATOM | 218 | O | ALA | A | 32 | 27.822 | 81.681 | −56.117 | 1.00 | 26.69 | A |
| ATOM | 219 | N | ARG | A | 33 | 27.443 | 83.802 | −56.738 | 1.00 | 30.53 | A |
| ATOM | 220 | CA | ARG | A | 33 | 27.595 | 83.520 | −58.155 | 1.00 | 31.94 | A |
| ATOM | 221 | CB | ARG | A | 33 | 26.300 | 82.923 | −58.720 | 1.00 | 33.20 | A |
| ATOM | 222 | CG | ARG | A | 33 | 25.041 | 83.718 | −58.408 | 1.00 | 34.07 | A |
| ATOM | 223 | CD | ARG | A | 33 | 23.857 | 83.216 | −59.233 | 1.00 | 34.60 | A |
| ATOM | 224 | NE | ARG | A | 33 | 22.660 | 84.030 | −59.026 | 1.00 | 34.36 | A |
| ATOM | 225 | CZ | ARG | A | 33 | 21.644 | 84.104 | −59.882 | 1.00 | 33.13 | A |
| ATOM | 226 | NH1 | ARG | A | 33 | 21.668 | 83.417 | −61.013 | 1.00 | 33.17 | A |
| ATOM | 227 | NH2 | ARG | A | 33 | 20.602 | 84.874 | −59.608 | 1.00 | 37.21 | A |
| ATOM | 228 | C | ARG | A | 33 | 27.975 | 84.783 | −58.921 | 1.00 | 31.83 | A |
| ATOM | 229 | O | ARG | A | 33 | 27.500 | 85.877 | −58.620 | 1.00 | 33.42 | A |
| ATOM | 230 | N | SER | A | 34 | 28.848 | 84.623 | −59.906 | 1.00 | 33.79 | A |
| ATOM | 231 | CA | SER | A | 34 | 29.303 | 85.739 | −60.728 | 1.00 | 33.09 | A |
| ATOM | 232 | CB | SER | A | 34 | 30.648 | 85.393 | −61.372 | 1.00 | 33.13 | A |
| ATOM | 233 | OG | SER | A | 34 | 30.912 | 86.238 | −62.480 | 1.00 | 39.33 | A |
| ATOM | 234 | C | SER | A | 34 | 28.298 | 86.079 | −61.823 | 1.00 | 32.23 | A |
| ATOM | 235 | O | SER | A | 34 | 27.543 | 85.215 | −62.273 | 1.00 | 28.50 | A |
| ATOM | 236 | N | PRO | A | 35 | 28.269 | 87.351 | −62.258 | 1.00 | 34.28 | A |
| ATOM | 237 | CD | PRO | A | 35 | 28.947 | 88.519 | −61.670 | 1.00 | 35.30 | A |
| ATOM | 238 | CA | PRO | A | 35 | 27.343 | 87.770 | −63.315 | 1.00 | 34.60 | A |
| ATOM | 239 | CB | PRO | A | 35 | 27.498 | 89.290 | −63.332 | 1.00 | 34.88 | A |
| ATOM | 240 | CG | PRO | A | 35 | 27.949 | 89.614 | −61.932 | 1.00 | 34.80 | A |
| ATOM | 241 | C | PRO | A | 35 | 27.807 | 87.138 | −64.626 | 1.00 | 36.69 | A |
| ATOM | 242 | O | PRO | A | 35 | 27.043 | 87.019 | −65.582 | 1.00 | 39.09 | A |
| ATOM | 243 | N | GLU | A | 36 | 29.077 | 86.741 | −64.648 | 1.00 | 36.95 | A |
| ATOM | 244 | CA | GLU | A | 36 | 29.696 | 86.116 | −65.811 | 1.00 | 37.99 | A |
| ATOM | 245 | CB | GLU | A | 36 | 31.213 | 86.059 | −65.615 | 1.00 | 43.07 | A |
| ATOM | 246 | CG | GLU | A | 36 | 31.968 | 85.306 | −66.704 | 1.00 | 49.48 | A |
| ATOM | 247 | CD | GLU | A | 36 | 31.796 | 85.919 | −68.086 | 1.00 | 52.72 | A |
| ATOM | 248 | OE1 | GLU | A | 36 | 32.464 | 85.444 | −69.031 | 1.00 | 54.76 | A |
| ATOM | 249 | OE2 | GLU | A | 36 | 30.995 | 86.870 | −68.233 | 1.00 | 53.49 | A |
| ATOM | 250 | C | GLU | A | 36 | 29.151 | 84.708 | −66.052 | 1.00 | 36.38 | A |
| ATOM | 251 | O | GLU | A | 36 | 28.871 | 84.331 | −67.187 | 1.00 | 35.87 | A |
| ATOM | 252 | N | SER | A | 37 | 29.014 | 83.935 | −64.980 | 1.00 | 32.12 | A |
| ATOM | 253 | CA | SER | A | 37 | 28.488 | 82.576 | −65.066 | 1.00 | 30.16 | A |
| ATOM | 254 | CB | SER | A | 37 | 29.621 | 81.559 | −64.918 | 1.00 | 31.67 | A |
| ATOM | 255 | OG | SER | A | 37 | 29.160 | 80.247 | −65.180 | 1.00 | 35.27 | A |
| ATOM | 256 | C | SER | A | 37 | 27.491 | 82.430 | −63.923 | 1.00 | 27.63 | A |
| ATOM | 257 | O | SER | A | 37 | 27.783 | 81.800 | −62.908 | 1.00 | 26.72 | A |
| ATOM | 258 | N | ASP | A | 38 | 26.311 | 83.021 | −64.100 | 1.00 | 25.93 | A |
| ATOM | 259 | CA | ASP | A | 38 | 25.278 | 83.017 | −63.068 | 1.00 | 23.68 | A |
| ATOM | 260 | CB | ASP | A | 38 | 24.588 | 84.385 | −63.039 | 1.00 | 20.47 | A |
| ATOM | 261 | CG | ASP | A | 38 | 23.881 | 84.722 | −64.349 | 1.00 | 25.63 | A |
| ATOM | 262 | OD1 | ASP | A | 38 | 23.323 | 85.843 | −64.444 | 1.00 | 26.06 | A |
| ATOM | 263 | OD2 | ASP | A | 38 | 23.873 | 83.882 | −65.279 | 1.00 | 19.34 | A |
| ATOM | 264 | C | ASP | A | 38 | 24.221 | 81.920 | −63.157 | 1.00 | 21.17 | A |
| ATOM | 265 | O | ASP | A | 38 | 23.155 | 82.037 | −62.554 | 1.00 | 20.85 | A |
| ATOM | 266 | N | SER | A | 39 | 24.506 | 80.857 | −63.897 | 1.00 | 19.47 | A |
| ATOM | 267 | CA | SER | A | 39 | 23.542 | 79.770 | −64.023 | 1.00 | 20.30 | A |
| ATOM | 268 | CB | SER | A | 39 | 23.769 | 79.000 | −65.326 | 1.00 | 20.35 | A |
| ATOM | 269 | OG | SER | A | 39 | 23.515 | 79.808 | −66.459 | 1.00 | 22.65 | A |
| ATOM | 270 | C | SER | A | 39 | 23.641 | 78.800 | −62.848 | 1.00 | 17.90 | A |
| ATOM | 271 | O | SER | A | 39 | 24.722 | 78.565 | −62.309 | 1.00 | 15.69 | A |
| ATOM | 272 | N | ILE | A | 40 | 22.506 | 78.248 | −62.445 | 1.00 | 15.17 | A |
| ATOM | 273 | CA | ILE | A | 40 | 22.496 | 77.283 | −61.357 | 1.00 | 15.55 | A |
| ATOM | 274 | CB | ILE | A | 40 | 21.230 | 77.407 | −60.482 | 1.00 | 20.40 | A |
| ATOM | 275 | CG2 | ILE | A | 40 | 21.264 | 76.363 | −59.371 | 1.00 | 20.05 | A |
| ATOM | 276 | CG1 | ILE | A | 40 | 21.111 | 78.826 | −59.915 | 1.00 | 22.56 | A |
| ATOM | 277 | CD1 | ILE | A | 40 | 22.296 | 79.270 | −59.097 | 1.00 | 22.27 | A |
| ATOM | 278 | C | ILE | A | 40 | 22.476 | 75.906 | −62.002 | 1.00 | 16.04 | A |
| ATOM | 279 | O | ILE | A | 40 | 21.792 | 75.699 | −63.002 | 1.00 | 15.16 | A |
| ATOM | 280 | N | GLN | A | 41 | 23.245 | 74.975 | −61.450 | 1.00 | 15.62 | A |
| ATOM | 281 | CA | GLN | A | 41 | 23.261 | 73.615 | −61.967 | 1.00 | 16.61 | A |
| ATOM | 282 | CB | GLN | A | 41 | 24.693 | 73.058 | −62.003 | 1.00 | 16.64 | A |
| ATOM | 283 | CG | GLN | A | 41 | 25.674 | 73.914 | −62.809 | 1.00 | 19.50 | A |
| ATOM | 284 | CD | GLN | A | 41 | 27.067 | 73.301 | −62.914 | 1.00 | 22.36 | A |
| ATOM | 285 | OE1 | GLN | A | 41 | 27.623 | 72.811 | −61.932 | 1.00 | 23.40 | A |
| ATOM | 286 | NE2 | GLN | A | 41 | 27.641 | 73.345 | −64.109 | 1.00 | 23.57 | A |
| ATOM | 287 | C | GLN | A | 41 | 22.393 | 72.801 | −61.010 | 1.00 | 18.11 | A |
| ATOM | 288 | O | GLN | A | 41 | 22.739 | 72.638 | −59.833 | 1.00 | 19.73 | A |
| ATOM | 289 | N | TRP | A | 42 | 21.251 | 72.326 | −61.503 | 1.00 | 17.02 | A |
| ATOM | 290 | CA | TRP | A | 42 | 20.336 | 71.533 | −60.682 | 1.00 | 16.00 | A |
| ATOM | 291 | CB | TRP | A | 42 | 18.875 | 71.917 | −60.941 | 1.00 | 14.16 | A |
| ATOM | 292 | CG | TRP | A | 42 | 18.451 | 73.235 | −60.354 | 1.00 | 16.21 | A |
| ATOM | 293 | CD2 | TRP | A | 42 | 18.110 | 73.495 | −58.987 | 1.00 | 14.23 | A |
| ATOM | 294 | CE2 | TRP | A | 42 | 17.795 | 74.867 | −58.890 | 1.00 | 15.32 | A |
| ATOM | 295 | CE3 | TRP | A | 42 | 18.041 | 72.701 | −57.834 | 1.00 | 14.86 | A |
| ATOM | 296 | CD1 | TRP | A | 42 | 18.331 | 74.425 | −61.012 | 1.00 | 13.74 | A |
| ATOM | 297 | NE1 | TRP | A | 42 | 17.937 | 75.409 | −60.140 | 1.00 | 17.53 | A |
| ATOM | 298 | CZ2 | TRP | A | 42 | 17.415 | 75.470 | −57.680 | 1.00 | 18.39 | A |
| ATOM | 299 | CZ3 | TRP | A | 42 | 17.664 | 73.297 | −56.630 | 1.00 | 17.51 | A |
| ATOM | 300 | CH2 | TRP | A | 42 | 17.355 | 74.672 | −56.566 | 1.00 | 18.33 | A |
| ATOM | 301 | C | TRP | A | 42 | 20.498 | 70.053 | −60.978 | 1.00 | 16.63 | A |
| ATOM | 302 | O | TRP | A | 42 | 20.592 | 69.651 | −62.142 | 1.00 | 17.82 | A |
| ATOM | 303 | N | PHE | A | 43 | 20.519 | 69.241 | −59.924 | 1.00 | 14.81 | A |
| ATOM | 304 | CA | PHE | A | 43 | 20.656 | 67.802 | −60.092 | 1.00 | 16.18 | A |
| ATOM | 305 | CB | PHE | A | 43 | 22.017 | 67.319 | −59.575 | 1.00 | 10.32 | A |
| ATOM | 306 | CG | PHE | A | 43 | 23.182 | 67.995 | −60.225 | 1.00 | 15.58 | A |
| ATOM | 307 | CD1 | PHE | A | 43 | 23.665 | 69.208 | −59.732 | 1.00 | 12.91 | A |
| ATOM | 308 | CD2 | PHE | A | 43 | 23.789 | 67.435 | −61.350 | 1.00 | 15.51 | A |
| ATOM | 309 | CE1 | PHE | A | 43 | 24.738 | 69.859 | −60.351 | 1.00 | 12.95 | A |
| ATOM | 310 | CE2 | PHE | A | 43 | 24.863 | 68.077 | −61.978 | 1.00 | 13.23 | A |
| ATOM | 311 | CZ | PHE | A | 43 | 25.339 | 69.292 | −61.477 | 1.00 | 12.53 | A |
| ATOM | 312 | C | PHE | A | 43 | 19.559 | 67.018 | −59.393 | 1.00 | 13.64 | A |
| ATOM | 313 | O | PHE | A | 43 | 19.213 | 67.302 | −58.248 | 1.00 | 15.64 | A |
| ATOM | 314 | N | HIS | A | 44 | 19.004 | 66.042 | −60.102 | 1.00 | 12.56 | A |
| ATOM | 315 | CA | HIS | A | 44 | 17.983 | 65.165 | −59.548 | 1.00 | 15.77 | A |
| ATOM | 316 | CB | HIS | A | 44 | 16.709 | 65.167 | −60.391 | 1.00 | 18.87 | A |
| ATOM | 317 | CG | HIS | A | 44 | 15.668 | 64.220 | −59.886 | 1.00 | 24.37 | A |
| ATOM | 318 | CD2 | HIS | A | 44 | 15.489 | 63.664 | −58.663 | 1.00 | 28.28 | A |
| ATOM | 319 | ND1 | HIS | A | 44 | 14.655 | 63.732 | −60.680 | 1.00 | 30.28 | A |
| ATOM | 320 | CE1 | HIS | A | 44 | 13.897 | 62.914 | −59.969 | 1.00 | 32.44 | A |
| ATOM | 321 | NE2 | HIS | A | 44 | 14.382 | 62.855 | −58.742 | 1.00 | 27.30 | A |
| ATOM | 322 | C | HIS | A | 44 | 18.627 | 63.791 | −59.615 | 1.00 | 14.40 | A |
| ATOM | 323 | O | HIS | A | 44 | 18.861 | 63.275 | −60.710 | 1.00 | 15.05 | A |
| ATOM | 324 | N | ASN | A | 45 | 18.913 | 63.213 | −58.450 | 1.00 | 11.69 | A |
| ATOM | 325 | CA | ASN | A | 45 | 19.585 | 61.920 | −58.353 | 1.00 | 12.79 | A |
| ATOM | 326 | CB | ASN | A | 45 | 18.723 | 60.791 | −58.925 | 1.00 | 12.97 | A |
| ATOM | 327 | CG | ASN | A | 45 | 17.574 | 60.414 | −58.008 | 1.00 | 18.46 | A |
| ATOM | 328 | OD1 | ASN | A | 45 | 17.641 | 60.613 | −56.794 | 1.00 | 13.77 | A |
| ATOM | 329 | ND2 | ASN | A | 45 | 16.519 | 59.853 | −58.584 | 1.00 | 17.79 | A |
| ATOM | 330 | C | ASN | A | 45 | 20.939 | 61.939 | −59.072 | 1.00 | 15.56 | A |
| ATOM | 331 | O | ASN | A | 45 | 21.354 | 60.931 | −59.649 | 1.00 | 15.61 | A |
| ATOM | 332 | N | GLY | A | 46 | 21.621 | 63.085 | −59.035 | 1.00 | 12.60 | A |
| ATOM | 333 | CA | GLY | A | 46 | 22.923 | 63.192 | −59.673 | 1.00 | 14.26 | A |
| ATOM | 334 | C | GLY | A | 46 | 22.904 | 63.539 | −61.154 | 1.00 | 15.79 | A |
| ATOM | 335 | O | GLY | A | 46 | 23.946 | 63.822 | −61.740 | 1.00 | 19.20 | A |
| ATOM | 336 | N | ASN | A | 47 | 21.723 | 63.529 | −61.757 | 1.00 | 16.39 | A |
| ATOM | 337 | CA | ASN | A | 47 | 21.573 | 63.846 | −63.178 | 1.00 | 18.08 | A |
| ATOM | 338 | CB | ASN | A | 47 | 20.528 | 62.931 | −63.808 | 1.00 | 19.30 | A |
| ATOM | 339 | CG | ASN | A | 47 | 20.912 | 61.474 | −63.728 | 1.00 | 22.03 | A |
| ATOM | 340 | OD1 | ASN | A | 47 | 20.048 | 60.595 | −63.688 | 1.00 | 23.67 | A |
| ATOM | 341 | ND2 | ASN | A | 47 | 22.214 | 61.205 | −63.714 | 1.00 | 19.79 | A |
| ATOM | 342 | C | ASN | A | 47 | 21.139 | 65.289 | −63.368 | 1.00 | 17.62 | A |
| ATOM | 343 | O | ASN | A | 47 | 20.159 | 65.733 | −62.768 | 1.00 | 18.05 | A |
| ATOM | 344 | N | LEU | A | 48 | 21.864 | 66.011 | −64.212 | 1.00 | 14.29 | A |
| ATOM | 345 | CA | LEU | A | 48 | 21.562 | 67.414 | −64.484 | 1.00 | 16.84 | A |
| ATOM | 346 | CB | LEU | A | 48 | 22.535 | 67.959 | −65.535 | 1.00 | 17.32 | A |
| ATOM | 347 | CG | LEU | A | 48 | 22.300 | 69.401 | −66.008 | 1.00 | 20.09 | A |
| ATOM | 348 | CD1 | LEU | A | 48 | 22.776 | 70.385 | −64.940 | 1.00 | 13.66 | A |
| ATOM | 349 | CD2 | LEU | A | 48 | 23.045 | 69.634 | −67.312 | 1.00 | 21.04 | A |
| ATOM | 350 | C | LEU | A | 48 | 20.128 | 67.642 | −64.978 | 1.00 | 16.59 | A |
| ATOM | 351 | O | LEU | A | 48 | 19.584 | 66.837 | −65.724 | 1.00 | 15.03 | A |
| ATOM | 352 | N | ILE | A | 49 | 19.518 | 68.738 | −64.537 | 1.00 | 17.73 | A |
| ATOM | 353 | CA | ILE | A | 49 | 18.177 | 69.106 | −64.984 | 1.00 | 19.75 | A |
| ATOM | 354 | CB | ILE | A | 49 | 17.279 | 69.542 | −63.814 | 1.00 | 21.33 | A |
| ATOM | 355 | CG2 | ILE | A | 49 | 15.864 | 69.794 | −64.319 | 1.00 | 19.91 | A |
| ATOM | 356 | CG1 | ILE | A | 49 | 17.274 | 68.465 | −62.729 | 1.00 | 20.03 | A |
| ATOM | 357 | CD1 | ILE | A | 49 | 16.511 | 68.864 | −61.477 | 1.00 | 18.59 | A |
| ATOM | 358 | C | ILE | A | 49 | 18.509 | 70.316 | −65.856 | 1.00 | 18.83 | A |
| ATOM | 359 | O | ILE | A | 49 | 18.521 | 71.456 | −65.385 | 1.00 | 18.60 | A |
| ATOM | 360 | N | PRO | A | 50 | 18.781 | 70.070 | −67.148 | 1.00 | 20.76 | A |
| ATOM | 361 | CD | PRO | A | 50 | 18.357 | 68.793 | −67.756 | 1.00 | 19.30 | A |
| ATOM | 362 | CA | PRO | A | 50 | 19.151 | 71.031 | −68.194 | 1.00 | 19.11 | A |
| ATOM | 363 | CB | PRO | A | 50 | 19.199 | 70.166 | −69.450 | 1.00 | 19.08 | A |
| ATOM | 364 | CG | PRO | A | 50 | 18.104 | 69.185 | −69.193 | 1.00 | 24.57 | A |
| ATOM | 365 | C | PRO | A | 50 | 18.337 | 72.297 | −68.401 | 1.00 | 18.44 | A |
| ATOM | 366 | O | PRO | A | 50 | 18.908 | 73.385 | −68.472 | 1.00 | 20.54 | A |
| ATOM | 367 | N | THR | A | 51 | 17.021 | 72.172 | −68.498 | 1.00 | 17.10 | A |
| ATOM | 368 | CA | THR | A | 51 | 16.173 | 73.337 | −68.739 | 1.00 | 22.04 | A |
| ATOM | 369 | CB | THR | A | 51 | 14.829 | 72.910 | −69.323 | 1.00 | 23.30 | A |
| ATOM | 370 | OG1 | THR | A | 51 | 14.201 | 71.978 | −68.436 | 1.00 | 27.29 | A |
| ATOM | 371 | CG2 | THR | A | 51 | 15.035 | 72.252 | −70.675 | 1.00 | 25.33 | A |
| ATOM | 372 | C | THR | A | 51 | 15.904 | 74.274 | −67.557 | 1.00 | 21.05 | A |
| ATOM | 373 | O | THR | A | 51 | 15.304 | 75.342 | −67.736 | 1.00 | 19.71 | A |
| ATOM | 374 | N | HIS | A | 52 | 16.338 | 73.889 | −66.360 | 1.00 | 15.46 | A |
| ATOM | 375 | CA | HIS | A | 52 | 16.142 | 74.739 | −65.185 | 1.00 | 15.44 | A |
| ATOM | 376 | CB | HIS | A | 52 | 15.396 | 73.973 | −64.097 | 1.00 | 15.87 | A |
| ATOM | 377 | CG | HIS | A | 52 | 14.005 | 73.599 | −64.493 | 1.00 | 17.50 | A |
| ATOM | 378 | CD2 | HIS | A | 52 | 13.488 | 72.425 | −64.925 | 1.00 | 14.80 | A |
| ATOM | 379 | ND1 | HIS | A | 52 | 12.979 | 74.518 | −64.550 | 1.00 | 15.69 | A |
| ATOM | 380 | CE1 | HIS | A | 52 | 11.888 | 73.925 | −65.002 | 1.00 | 19.96 | A |
| ATOM | 381 | NE2 | HIS | A | 52 | 12.170 | 72.655 | −65.238 | 1.00 | 22.13 | A |
| ATOM | 382 | C | HIS | A | 52 | 17.492 | 75.207 | −64.680 | 1.00 | 15.52 | A |
| ATOM | 383 | O | HIS | A | 52 | 18.287 | 74.414 | −64.173 | 1.00 | 17.01 | A |
| ATOM | 384 | N | THR | A | 53 | 17.748 | 76.502 | −64.818 | 1.00 | 13.36 | A |
| ATOM | 385 | CA | THR | A | 53 | 19.023 | 77.058 | −64.410 | 1.00 | 14.40 | A |
| ATOM | 386 | CB | THR | A | 53 | 19.871 | 77.389 | −65.655 | 1.00 | 17.69 | A |
| ATOM | 387 | OG1 | THR | A | 53 | 19.217 | 78.409 | −66.422 | 1.00 | 16.73 | A |
| ATOM | 388 | CG2 | THR | A | 53 | 20.036 | 76.141 | −66.536 | 1.00 | 13.46 | A |
| ATOM | 389 | C | THR | A | 53 | 18.906 | 78.306 | −63.549 | 1.00 | 16.04 | A |
| ATOM | 390 | O | THR | A | 53 | 19.841 | 79.103 | −63.485 | 1.00 | 18.61 | A |
| ATOM | 391 | N | GLN | A | 54 | 17.767 | 78.481 | −62.887 | 1.00 | 15.88 | A |
| ATOM | 392 | CA | GLN | A | 54 | 17.568 | 79.652 | −62.037 | 1.00 | 18.40 | A |
| ATOM | 393 | CB | GLN | A | 54 | 16.188 | 80.259 | −62.286 | 1.00 | 22.88 | A |
| ATOM | 394 | CG | GLN | A | 54 | 15.960 | 80.749 | −63.709 | 1.00 | 31.13 | A |
| ATOM | 395 | CD | GLN | A | 54 | 16.931 | 81.843 | −64.114 | 1.00 | 35.31 | A |
| ATOM | 396 | OE1 | GLN | A | 54 | 17.115 | 82.824 | −63.392 | 1.00 | 36.98 | A |
| ATOM | 397 | NE2 | GLN | A | 54 | 17.553 | 81.683 | −65.281 | 1.00 | 42.07 | A |
| ATOM | 398 | C | GLN | A | 54 | 17.714 | 79.315 | −60.556 | 1.00 | 18.27 | A |
| ATOM | 399 | O | GLN | A | 54 | 17.628 | 78.148 | −60.156 | 1.00 | 17.81 | A |
| ATOM | 400 | N | PRO | A | 55 | 17.952 | 80.337 | −59.719 | 1.00 | 18.23 | A |
| ATOM | 401 | CD | PRO | A | 55 | 18.252 | 81.740 | −60.067 | 1.00 | 17.34 | A |
| ATOM | 402 | CA | PRO | A | 55 | 18.105 | 80.116 | −58.277 | 1.00 | 16.18 | A |
| ATOM | 403 | CB | PRO | A | 55 | 18.130 | 81.535 | −57.721 | 1.00 | 15.55 | A |
| ATOM | 404 | CG | PRO | A | 55 | 18.902 | 82.268 | −58.789 | 1.00 | 14.32 | A |
| ATOM | 405 | C | PRO | A | 55 | 16.987 | 79.262 | −57.686 | 1.00 | 15.87 | A |
| ATOM | 406 | O | PRO | A | 55 | 17.223 | 78.472 | −56.773 | 1.00 | 17.38 | A |
| ATOM | 407 | N | SER | A | 56 | 15.773 | 79.418 | −58.206 | 1.00 | 15.98 | A |
| ATOM | 408 | CA | SER | A | 56 | 14.635 | 78.634 | −57.725 | 1.00 | 15.75 | A |
| ATOM | 409 | CB | SER | A | 56 | 13.491 | 79.548 | −57.261 | 1.00 | 13.79 | A |
| ATOM | 410 | OG | SER | A | 56 | 13.849 | 80.267 | −56.094 | 1.00 | 14.31 | A |
| ATOM | 411 | C | SER | A | 56 | 14.106 | 77.694 | −58.799 | 1.00 | 17.57 | A |
| ATOM | 412 | O | SER | A | 56 | 13.982 | 78.072 | −59.967 | 1.00 | 21.26 | A |
| ATOM | 413 | N | TYR | A | 57 | 13.800 | 76.468 | −58.387 | 1.00 | 16.19 | A |
| ATOM | 414 | CA | TYR | A | 57 | 13.254 | 75.444 | −59.271 | 1.00 | 19.00 | A |
| ATOM | 415 | CB | TYR | A | 57 | 14.194 | 74.229 | −59.294 | 1.00 | 19.44 | A |
| ATOM | 416 | CG | TYR | A | 57 | 13.669 | 73.058 | −60.079 | 1.00 | 20.80 | A |
| ATOM | 417 | CD1 | TYR | A | 57 | 12.895 | 73.253 | −61.219 | 1.00 | 23.22 | A |
| ATOM | 418 | CE1 | TYR | A | 57 | 12.404 | 72.178 | −61.943 | 1.00 | 25.34 | A |
| ATOM | 419 | CD2 | TYR | A | 57 | 13.945 | 71.751 | −59.683 | 1.00 | 23.28 | A |
| ATOM | 420 | CE2 | TYR | A | 57 | 13.462 | 70.667 | −60.404 | 1.00 | 24.44 | A |
| ATOM | 421 | CZ | TYR | A | 57 | 12.689 | 70.888 | −61.533 | 1.00 | 26.83 | A |
| ATOM | 422 | OH | TYR | A | 57 | 12.181 | 69.824 | −62.247 | 1.00 | 30.72 | A |
| ATOM | 423 | C | TYR | A | 57 | 11.857 | 75.071 | −58.734 | 1.00 | 17.79 | A |
| ATOM | 424 | O | TYR | A | 57 | 11.721 | 74.528 | −57.638 | 1.00 | 16.09 | A |
| ATOM | 425 | N | ARG | A | 58 | 10.833 | 75.372 | −59.524 | 1.00 | 18.09 | A |
| ATOM | 426 | CA | ARG | A | 58 | 9.435 | 75.153 | −59.151 | 1.00 | 22.44 | A |
| ATOM | 427 | CB | ARG | A | 58 | 8.696 | 76.485 | −59.314 | 1.00 | 23.61 | A |
| ATOM | 428 | CG | ARG | A | 58 | 7.280 | 76.532 | −58.789 | 1.00 | 33.50 | A |
| ATOM | 429 | CD | ARG | A | 58 | 6.636 | 77.866 | −59.162 | 1.00 | 34.31 | A |
| ATOM | 430 | NE | ARG | A | 58 | 5.316 | 78.047 | −58.559 | 1.00 | 36.63 | A |
| ATOM | 431 | CZ | ARG | A | 58 | 5.114 | 78.307 | −57.272 | 1.00 | 35.63 | A |
| ATOM | 432 | NH1 | ARG | A | 58 | 6.150 | 78.420 | −56.451 | 1.00 | 32.49 | A |
| ATOM | 433 | NH2 | ARG | A | 58 | 3.877 | 78.443 | −56.803 | 1.00 | 34.09 | A |
| ATOM | 434 | C | ARG | A | 58 | 8.722 | 74.060 | −59.962 | 1.00 | 20.65 | A |
| ATOM | 435 | O | ARG | A | 58 | 8.906 | 73.955 | −61.172 | 1.00 | 22.15 | A |
| ATOM | 436 | N | PHE | A | 59 | 7.909 | 73.247 | −59.291 | 1.00 | 21.15 | A |
| ATOM | 437 | CA | PHE | A | 59 | 7.165 | 72.187 | −59.970 | 1.00 | 18.84 | A |
| ATOM | 438 | CB | PHE | A | 59 | 8.109 | 71.058 | −60.418 | 1.00 | 20.68 | A |
| ATOM | 439 | CG | PHE | A | 59 | 8.831 | 70.369 | −59.284 | 1.00 | 18.66 | A |
| ATOM | 440 | CD1 | PHE | A | 59 | 10.093 | 70.796 | −58.881 | 1.00 | 21.19 | A |
| ATOM | 441 | CD2 | PHE | A | 59 | 8.244 | 69.299 | −58.615 | 1.00 | 17.58 | A |
| ATOM | 442 | CE1 | PHE | A | 59 | 10.762 | 70.166 | −57.828 | 1.00 | 21.70 | A |
| ATOM | 443 | CE2 | PHE | A | 59 | 8.900 | 68.662 | −57.562 | 1.00 | 18.95 | A |
| ATOM | 444 | CZ | PHE | A | 59 | 10.162 | 69.095 | −57.167 | 1.00 | 18.27 | A |
| ATOM | 445 | C | PHE | A | 59 | 6.046 | 71.576 | −59.130 | 1.00 | 19.33 | A |
| ATOM | 446 | O | PHE | A | 59 | 5.970 | 71.791 | −57.917 | 1.00 | 20.07 | A |
| ATOM | 447 | N | LYS | A | 60 | 5.182 | 70.813 | −59.800 | 1.00 | 20.76 | A |
| ATOM | 448 | CA | LYS | A | 60 | 4.069 | 70.119 | −59.161 | 1.00 | 22.83 | A |
| ATOM | 449 | CB | LYS | A | 60 | 2.807 | 70.202 | −60.019 | 1.00 | 24.97 | A |
| ATOM | 450 | CG | LYS | A | 60 | 2.065 | 71.525 | −59.919 | 1.00 | 28.94 | A |
| ATOM | 451 | CD | LYS | A | 60 | 0.603 | 71.296 | −59.534 | 1.00 | 34.46 | A |
| ATOM | 452 | CE | LYS | A | 60 | 0.105 | 70.346 | −60.509 | 1.00 | 36.54 | A |
| ATOM | 453 | NZ | LYS | A | 60 | −1.494 | 70.007 | −60.066 | 1.00 | 37.79 | A |
| ATOM | 454 | C | LYS | A | 60 | 4.476 | 68.664 | −58.984 | 1.00 | 23.01 | A |
| ATOM | 455 | O | LYS | A | 60 | 4.636 | 67.927 | −59.957 | 1.00 | 25.26 | A |
| ATOM | 456 | N | ALA | A | 61 | 4.643 | 68.262 | −57.730 | 1.00 | 24.13 | A |
| ATOM | 457 | CA | ALA | A | 61 | 5.078 | 66.912 | −57.387 | 1.00 | 26.61 | A |
| ATOM | 458 | CB | ALA | A | 61 | 5.015 | 66.724 | −55.867 | 1.00 | 22.53 | A |
| ATOM | 459 | C | ALA | A | 61 | 4.365 | 65.749 | −58.074 | 1.00 | 26.01 | A |
| ATOM | 460 | O | ALA | A | 61 | 3.142 | 65.655 | −58.067 | 1.00 | 24.84 | A |
| ATOM | 461 | N | ASN | A | 62 | 5.166 | 64.874 | −58.670 | 1.00 | 30.63 | A |
| ATOM | 462 | CA | ASN | A | 62 | 4.696 | 63.657 | −59.331 | 1.00 | 34.30 | A |
| ATOM | 463 | CB | ASN | A | 62 | 5.129 | 63.594 | −60.796 | 1.00 | 37.21 | A |
| ATOM | 464 | CG | ASN | A | 62 | 4.623 | 64.753 | −61.608 | 1.00 | 41.94 | A |
| ATOM | 465 | OD1 | ASN | A | 62 | 3.418 | 65.012 | −61.662 | 1.00 | 45.50 | A |
| ATOM | 466 | ND2 | ASN | A | 62 | 5.541 | 65.461 | −62.259 | 1.00 | 43.53 | A |
| ATOM | 467 | C | ASN | A | 62 | 5.457 | 62.582 | −58.586 | 1.00 | 35.79 | A |
| ATOM | 468 | O | ASN | A | 62 | 6.325 | 62.894 | −57.767 | 1.00 | 35.52 | A |
| ATOM | 469 | N | ASN | A | 63 | 5.154 | 61.319 | −58.857 | 1.00 | 38.50 | A |
| ATOM | 470 | CA | ASN | A | 63 | 5.886 | 60.257 | −58.189 | 1.00 | 40.03 | A |
| ATOM | 471 | CB | ASN | A | 63 | 5.255 | 58.893 | −58.473 | 1.00 | 44.80 | A |
| ATOM | 472 | CG | ASN | A | 63 | 5.454 | 58.446 | −59.909 | 1.00 | 51.46 | A |
| ATOM | 473 | OD1 | ASN | A | 63 | 4.874 | 59.014 | −60.840 | 1.00 | 54.68 | A |
| ATOM | 474 | ND2 | ASN | A | 63 | 6.287 | 57.424 | −60.098 | 1.00 | 52.24 | A |
| ATOM | 475 | C | ASN | A | 63 | 7.306 | 60.303 | −58.752 | 1.00 | 39.00 | A |
| ATOM | 476 | O | ASN | A | 63 | 8.276 | 60.031 | −58.050 | 1.00 | 39.32 | A |
| ATOM | 477 | N | ASN | A | 64 | 7.418 | 60.681 | −60.022 | 1.00 | 37.34 | A |
| ATOM | 478 | CA | ASN | A | 64 | 8.712 | 60.758 | −60.693 | 1.00 | 37.85 | A |
| ATOM | 479 | CB | ASN | A | 64 | 8.510 | 60.917 | −62.203 | 1.00 | 42.39 | A |
| ATOM | 480 | CG | ASN | A | 64 | 7.579 | 62.063 | −62.544 | 1.00 | 50.58 | A |
| ATOM | 481 | OD1 | ASN | A | 64 | 7.846 | 63.221 | −62.203 | 1.00 | 52.85 | A |
| ATOM | 482 | ND2 | ASN | A | 64 | 6.473 | 61.747 | −63.219 | 1.00 | 54.77 | A |
| ATOM | 483 | C | ASN | A | 64 | 9.612 | 61.881 | −60.180 | 1.00 | 32.56 | A |
| ATOM | 484 | O | ASN | A | 64 | 10.767 | 61.996 | −60.598 | 1.00 | 27.88 | A |
| ATOM | 485 | N | ASP | A | 65 | 9.088 | 62.716 | −59.289 | 1.00 | 27.14 | A |
| ATOM | 486 | CA | ASP | A | 65 | 9.884 | 63.809 | −58.740 | 1.00 | 23.11 | A |
| ATOM | 487 | CB | ASP | A | 65 | 9.021 | 65.053 | −58.498 | 1.00 | 22.42 | A |
| ATOM | 488 | CG | ASP | A | 65 | 8.587 | 65.720 | −59.780 | 1.00 | 20.05 | A |
| ATOM | 489 | OD1 | ASP | A | 65 | 9.444 | 65.939 | −60.665 | 1.00 | 23.22 | A |
| ATOM | 490 | OD2 | ASP | A | 65 | 7.389 | 66.038 | −59.897 | 1.00 | 22.06 | A |
| ATOM | 491 | C | ASP | A | 65 | 10.565 | 63.404 | −57.438 | 1.00 | 18.68 | A |
| ATOM | 492 | O | ASP | A | 65 | 11.367 | 64.154 | −56.896 | 1.00 | 20.40 | A |
| ATOM | 493 | N | SER | A | 66 | 10.236 | 62.221 | −56.931 | 1.00 | 16.92 | A |
| ATOM | 494 | CA | SER | A | 66 | 10.848 | 61.736 | −55.700 | 1.00 | 15.22 | A |
| ATOM | 495 | CB | SER | A | 66 | 10.219 | 60.407 | −55.281 | 1.00 | 14.33 | A |
| ATOM | 496 | OG | SER | A | 66 | 8.870 | 60.580 | −54.893 | 1.00 | 15.27 | A |
| ATOM | 497 | C | SER | A | 66 | 12.341 | 61.534 | −55.935 | 1.00 | 13.74 | A |
| ATOM | 498 | O | SER | A | 66 | 12.771 | 61.340 | −57.072 | 1.00 | 14.60 | A |
| ATOM | 499 | N | GLY | A | 67 | 13.123 | 61.571 | −54.860 | 1.00 | 13.08 | A |
| ATOM | 500 | CA | GLY | A | 67 | 14.560 | 61.379 | −54.977 | 1.00 | 8.93 | A |
| ATOM | 501 | C | GLY | A | 67 | 15.344 | 62.473 | −54.275 | 1.00 | 12.12 | A |
| ATOM | 502 | O | GLY | A | 67 | 14.798 | 63.220 | −53.457 | 1.00 | 8.57 | A |
| ATOM | 503 | N | GLU | A | 68 | 16.632 | 62.569 | −54.586 | 1.00 | 13.75 | A |
| ATOM | 504 | CA | GLU | A | 68 | 17.468 | 63.588 | −53.970 | 1.00 | 16.34 | A |
| ATOM | 505 | CB | GLU | A | 68 | 18.828 | 63.042 | −53.558 | 1.00 | 16.00 | A |
| ATOM | 506 | CG | GLU | A | 68 | 18.850 | 61.663 | −52.977 | 1.00 | 22.51 | A |
| ATOM | 507 | CD | GLU | A | 68 | 20.207 | 61.358 | −52.386 | 1.00 | 18.07 | A |
| ATOM | 508 | OE1 | GLU | A | 68 | 21.213 | 61.837 | −52.956 | 1.00 | 16.43 | A |
| ATOM | 509 | OE2 | GLU | A | 68 | 20.264 | 60.644 | −51.366 | 1.00 | 17.90 | A |
| ATOM | 510 | C | GLU | A | 68 | 17.742 | 64.728 | −54.926 | 1.00 | 16.21 | A |
| ATOM | 511 | O | GLU | A | 68 | 17.839 | 64.548 | −56.147 | 1.00 | 14.10 | A |
| ATOM | 512 | N | TYR | A | 69 | 17.895 | 65.911 | −54.354 | 1.00 | 16.04 | A |
| ATOM | 513 | CA | TYR | A | 69 | 18.211 | 67.068 | −55.152 | 1.00 | 15.52 | A |
| ATOM | 514 | CB | TYR | A | 69 | 17.053 | 68.064 | −55.122 | 1.00 | 13.07 | A |
| ATOM | 515 | CG | TYR | A | 69 | 15.849 | 67.560 | −55.878 | 1.00 | 16.47 | A |
| ATOM | 516 | CD1 | TYR | A | 69 | 14.902 | 66.744 | −55.260 | 1.00 | 16.93 | A |
| ATOM | 517 | CE1 | TYR | A | 69 | 13.826 | 66.217 | −55.979 | 1.00 | 16.69 | A |
| ATOM | 518 | CD2 | TYR | A | 69 | 15.688 | 67.846 | −57.236 | 1.00 | 16.67 | A |
| ATOM | 519 | CE2 | TYR | A | 69 | 14.619 | 67.324 | −57.964 | 1.00 | 15.22 | A |
| ATOM | 520 | CZ | TYR | A | 69 | 13.695 | 66.507 | −57.331 | 1.00 | 16.84 | A |
| ATOM | 521 | OH | TYR | A | 69 | 12.667 | 65.946 | −58.060 | 1.00 | 15.69 | A |
| ATOM | 522 | C | TYR | A | 69 | 19.487 | 67.701 | −54.635 | 1.00 | 13.06 | A |
| ATOM | 523 | O | TYR | A | 69 | 19.755 | 67.698 | −53.433 | 1.00 | 15.71 | A |
| ATOM | 524 | N | THR | A | 70 | 20.296 | 68.192 | −55.561 | 1.00 | 11.91 | A |
| ATOM | 525 | CA | THR | A | 70 | 21.530 | 68.877 | −55.221 | 1.00 | 11.47 | A |
| ATOM | 526 | CB | THR | A | 70 | 22.770 | 67.960 | −55.343 | 1.00 | 12.38 | A |
| ATOM | 527 | OG1 | THR | A | 70 | 22.693 | 67.199 | −56.556 | 1.00 | 12.16 | A |
| ATOM | 528 | CG2 | THR | A | 70 | 22.865 | 67.018 | −54.141 | 1.00 | 11.66 | A |
| ATOM | 529 | C | THR | A | 70 | 21.636 | 70.018 | −56.215 | 1.00 | 13.74 | A |
| ATOM | 530 | O | THR | A | 70 | 21.039 | 69.962 | −57.295 | 1.00 | 16.11 | A |
| ATOM | 531 | N | CYS | A | 71 | 22.377 | 71.057 | −55.850 | 1.00 | 12.95 | A |
| ATOM | 532 | CA | CYS | A | 71 | 22.545 | 72.212 | −56.723 | 1.00 | 12.99 | A |
| ATOM | 533 | C | CYS | A | 71 | 23.901 | 72.856 | −56.476 | 1.00 | 13.24 | A |
| ATOM | 534 | O | CYS | A | 71 | 24.508 | 72.664 | −55.421 | 1.00 | 13.34 | A |
| ATOM | 535 | CB | CYS | A | 71 | 21.435 | 73.231 | −56.464 | 1.00 | 12.68 | A |
| ATOM | 536 | SG | CYS | A | 71 | 21.602 | 74.150 | −54.897 | 1.00 | 15.59 | A |
| ATOM | 537 | N | GLN | A | 72 | 24.379 | 73.612 | −57.456 | 1.00 | 12.72 | A |
| ATOM | 538 | CA | GLN | A | 72 | 25.666 | 74.282 | −57.334 | 1.00 | 13.89 | A |
| ATOM | 539 | CB | GLN | A | 72 | 26.784 | 73.344 | −57.806 | 1.00 | 11.97 | A |
| ATOM | 540 | CG | GLN | A | 72 | 28.175 | 73.949 | −57.738 | 1.00 | 19.05 | A |
| ATOM | 541 | CD | GLN | A | 72 | 29.277 | 72.900 | −57.720 | 1.00 | 22.99 | A |
| ATOM | 542 | OE1 | GLN | A | 72 | 29.099 | 71.789 | −58.219 | 1.00 | 22.39 | A |
| ATOM | 543 | NE2 | GLN | A | 72 | 30.431 | 73.258 | −57.153 | 1.00 | 21.90 | A |
| ATOM | 544 | C | GLN | A | 72 | 25.673 | 75.568 | −58.155 | 1.00 | 15.86 | A |
| ATOM | 545 | O | GLN | A | 72 | 24.919 | 75.705 | −59.123 | 1.00 | 15.43 | A |
| ATOM | 546 | N | THR | A | 73 | 26.508 | 76.519 | −57.752 | 1.00 | 16.73 | A |
| ATOM | 547 | CA | THR | A | 73 | 26.624 | 77.783 | −58.472 | 1.00 | 18.91 | A |
| ATOM | 548 | CB | THR | A | 73 | 26.224 | 78.987 | −57.606 | 1.00 | 17.41 | A |
| ATOM | 549 | OG1 | THR | A | 73 | 27.151 | 79.125 | −56.522 | 1.00 | 19.74 | A |
| ATOM | 550 | CG2 | THR | A | 73 | 24.816 | 78.801 | −57.053 | 1.00 | 14.98 | A |
| ATOM | 551 | C | THR | A | 73 | 28.082 | 77.939 | −58.861 | 1.00 | 17.98 | A |
| ATOM | 552 | O | THR | A | 73 | 28.933 | 77.171 | −58.419 | 1.00 | 20.03 | A |
| ATOM | 553 | N | GLY | A | 74 | 28.373 | 78.937 | −59.678 | 1.00 | 17.08 | A |
| ATOM | 554 | CA | GLY | A | 74 | 29.741 | 79.140 | −60.111 | 1.00 | 20.82 | A |
| ATOM | 555 | C | GLY | A | 74 | 30.781 | 79.359 | −59.026 | 1.00 | 21.44 | A |
| ATOM | 556 | O | GLY | A | 74 | 31.934 | 78.985 | −59.211 | 1.00 | 21.79 | A |
| ATOM | 557 | N | GLN | A | 75 | 30.394 | 79.944 | −57.895 | 1.00 | 23.16 | A |
| ATOM | 558 | CA | GLN | A | 75 | 31.360 | 80.213 | −56.830 | 1.00 | 23.55 | A |
| ATOM | 559 | CB | GLN | A | 75 | 31.355 | 81.713 | −56.501 | 1.00 | 23.39 | A |
| ATOM | 560 | CG | GLN | A | 75 | 31.773 | 82.586 | −57.685 | 1.00 | 19.49 | A |
| ATOM | 561 | CD | GLN | A | 75 | 31.835 | 84.066 | −57.355 | 1.00 | 21.27 | A |
| ATOM | 562 | OE1 | GLN | A | 75 | 30.831 | 84.684 | −57.007 | 1.00 | 20.84 | A |
| ATOM | 563 | NE2 | GLN | A | 75 | 33.021 | 84.643 | −57.472 | 1.00 | 22.04 | A |
| ATOM | 564 | C | GLN | A | 75 | 31.218 | 79.397 | −55.541 | 1.00 | 24.98 | A |
| ATOM | 565 | O | GLN | A | 75 | 31.786 | 79.762 | −54.509 | 1.00 | 25.85 | A |
| ATOM | 566 | N | THR | A | 76 | 30.474 | 78.295 | −55.598 | 1.00 | 22.90 | A |
| ATOM | 567 | CA | THR | A | 76 | 30.300 | 77.437 | −54.431 | 1.00 | 20.07 | A |
| ATOM | 568 | CB | THR | A | 76 | 28.890 | 77.562 | −53.826 | 1.00 | 20.62 | A |
| ATOM | 569 | OG1 | THR | A | 76 | 27.927 | 77.031 | −54.747 | 1.00 | 22.96 | A |
| ATOM | 570 | CG2 | THR | A | 76 | 28.562 | 79.012 | −53.535 | 1.00 | 20.96 | A |
| ATOM | 571 | C | THR | A | 76 | 30.499 | 75.980 | −54.820 | 1.00 | 19.79 | A |
| ATOM | 572 | O | THR | A | 76 | 30.639 | 75.653 | −56.001 | 1.00 | 16.94 | A |
| ATOM | 573 | N | SER | A | 77 | 30.511 | 75.106 | −53.817 | 1.00 | 19.32 | A |
| ATOM | 574 | CA | SER | A | 77 | 30.668 | 73.673 | −54.051 | 1.00 | 17.43 | A |
| ATOM | 575 | CB | SER | A | 77 | 31.486 | 73.043 | −52.921 | 1.00 | 22.53 | A |
| ATOM | 576 | OG | SER | A | 77 | 32.688 | 73.761 | −52.706 | 1.00 | 24.60 | A |
| ATOM | 577 | C | SER | A | 77 | 29.276 | 73.043 | −54.097 | 1.00 | 16.57 | A |
| ATOM | 578 | O | SER | A | 77 | 28.288 | 73.683 | −53.721 | 1.00 | 17.07 | A |
| ATOM | 579 | N | LEU | A | 78 | 29.197 | 71.798 | −54.559 | 1.00 | 14.19 | A |
| ATOM | 580 | CA | LEU | A | 78 | 27.918 | 71.090 | −54.650 | 1.00 | 15.15 | A |
| ATOM | 581 | CB | LEU | A | 78 | 28.132 | 69.674 | −55.202 | 1.00 | 12.95 | A |
| ATOM | 582 | CG | LEU | A | 78 | 26.904 | 68.783 | −55.425 | 1.00 | 12.82 | A |
| ATOM | 583 | CD1 | LEU | A | 78 | 26.030 | 69.355 | −56.530 | 1.00 | 10.22 | A |
| ATOM | 584 | CD2 | LEU | A | 78 | 27.361 | 67.369 | −55.789 | 1.00 | 10.27 | A |
| ATOM | 585 | C | LEU | A | 78 | 27.276 | 71.019 | −53.270 | 1.00 | 15.00 | A |
| ATOM | 586 | O | LEU | A | 78 | 27.926 | 70.656 | −52.293 | 1.00 | 17.53 | A |
| ATOM | 587 | N | SER | A | 79 | 25.999 | 71.367 | −53.195 | 1.00 | 16.57 | A |
| ATOM | 588 | CA | SER | A | 79 | 25.266 | 71.363 | −51.927 | 1.00 | 16.66 | A |
| ATOM | 589 | CB | SER | A | 79 | 23.911 | 72.036 | −52.105 | 1.00 | 15.43 | A |
| ATOM | 590 | OG | SER | A | 79 | 23.031 | 71.162 | −52.799 | 1.00 | 13.56 | A |
| ATOM | 591 | C | SER | A | 79 | 25.009 | 69.956 | −51.409 | 1.00 | 14.64 | A |
| ATOM | 592 | O | SER | A | 79 | 25.076 | 68.990 | −52.164 | 1.00 | 14.09 | A |
| ATOM | 593 | N | ASP | A | 80 | 24.727 | 69.852 | −50.113 | 1.00 | 15.69 | A |
| ATOM | 594 | CA | ASP | A | 80 | 24.392 | 68.568 | −49.515 | 1.00 | 15.97 | A |
| ATOM | 595 | CB | ASP | A | 80 | 24.260 | 68.662 | −47.987 | 1.00 | 18.12 | A |
| ATOM | 596 | CG | ASP | A | 80 | 25.601 | 68.829 | −47.288 | 1.00 | 19.75 | A |
| ATOM | 597 | OD1 | ASP | A | 80 | 26.552 | 68.101 | −47.629 | 1.00 | 18.25 | A |
| ATOM | 598 | OD2 | ASP | A | 80 | 25.704 | 69.685 | −46.386 | 1.00 | 23.89 | A |
| ATOM | 599 | C | ASP | A | 80 | 23.028 | 68.279 | −50.124 | 1.00 | 17.05 | A |
| ATOM | 600 | O | ASP | A | 80 | 22.274 | 69.203 | −50.452 | 1.00 | 18.29 | A |
| ATOM | 601 | N | PRO | A | 81 | 22.687 | 67.000 | −50.283 | 1.00 | 14.58 | A |
| ATOM | 602 | CD | PRO | A | 81 | 23.515 | 65.806 | −50.040 | 1.00 | 15.78 | A |
| ATOM | 603 | CA | PRO | A | 81 | 21.399 | 66.637 | −50.870 | 1.00 | 15.60 | A |
| ATOM | 604 | CB | PRO | A | 81 | 21.579 | 65.157 | −51.196 | 1.00 | 16.51 | A |
| ATOM | 605 | CG | PRO | A | 81 | 22.491 | 64.690 | −50.105 | 1.00 | 17.60 | A |
| ATOM | 606 | C | PRO | A | 81 | 20.160 | 66.882 | −50.022 | 1.00 | 16.72 | A |
| ATOM | 607 | O | PRO | A | 81 | 20.221 | 66.990 | −48.802 | 1.00 | 17.75 | A |
| ATOM | 608 | N | VAL | A | 82 | 19.027 | 66.981 | −50.700 | 1.00 | 16.38 | A |
| ATOM | 609 | CA | VAL | A | 82 | 17.750 | 67.153 | −50.036 | 1.00 | 17.54 | A |
| ATOM | 610 | CB | VAL | A | 82 | 17.107 | 68.498 | −50.388 | 1.00 | 17.35 | A |
| ATOM | 611 | CG1 | VAL | A | 82 | 15.749 | 68.609 | −49.733 | 1.00 | 16.46 | A |
| ATOM | 612 | CG2 | VAL | A | 82 | 18.008 | 69.628 | −49.930 | 1.00 | 21.27 | A |
| ATOM | 613 | C | VAL | A | 82 | 16.901 | 66.008 | −50.576 | 1.00 | 16.45 | A |
| ATOM | 614 | O | VAL | A | 82 | 16.979 | 65.683 | −51.760 | 1.00 | 14.59 | A |
| ATOM | 615 | N | HIS | A | 83 | 16.111 | 65.387 | −49.708 | 1.00 | 16.29 | A |
| ATOM | 616 | CA | HIS | A | 83 | 15.272 | 64.267 | −50.108 | 1.00 | 15.77 | A |
| ATOM | 617 | CB | HIS | A | 83 | 15.510 | 63.090 | −49.166 | 1.00 | 18.16 | A |
| ATOM | 618 | CG | HIS | A | 83 | 16.958 | 62.802 | −48.927 | 1.00 | 22.63 | A |
| ATOM | 619 | CD2 | HIS | A | 83 | 17.780 | 63.159 | −47.912 | 1.00 | 22.27 | A |
| ATOM | 620 | ND1 | HIS | A | 83 | 17.740 | 62.113 | −49.828 | 1.00 | 24.95 | A |
| ATOM | 621 | CE1 | HIS | A | 83 | 18.980 | 62.059 | −49.379 | 1.00 | 24.85 | A |
| ATOM | 622 | NE2 | HIS | A | 83 | 19.031 | 62.687 | −48.220 | 1.00 | 26.99 | A |
| ATOM | 623 | C | HIS | A | 83 | 13.794 | 64.628 | −50.112 | 1.00 | 17.02 | A |
| ATOM | 624 | O | HIS | A | 83 | 13.229 | 65.028 | −49.097 | 1.00 | 18.30 | A |
| ATOM | 625 | N | LEU | A | 84 | 13.171 | 64.481 | −51.271 | 1.00 | 17.43 | A |
| ATOM | 626 | CA | LEU | A | 84 | 11.757 | 64.765 | −51.409 | 1.00 | 16.78 | A |
| ATOM | 627 | CB | LEU | A | 84 | 11.519 | 65.713 | −52.590 | 1.00 | 19.88 | A |
| ATOM | 628 | CG | LEU | A | 84 | 10.077 | 66.184 | −52.805 | 1.00 | 16.91 | A |
| ATOM | 629 | CD1 | LEU | A | 84 | 9.669 | 67.132 | −51.662 | 1.00 | 17.14 | A |
| ATOM | 630 | CD2 | LEU | A | 84 | 9.976 | 66.876 | −54.142 | 1.00 | 14.22 | A |
| ATOM | 631 | C | LEU | A | 84 | 11.042 | 63.444 | −51.660 | 1.00 | 16.64 | A |
| ATOM | 632 | O | LEU | A | 84 | 11.457 | 62.658 | −52.513 | 1.00 | 17.61 | A |
| ATOM | 633 | N | THR | A | 85 | 9.982 | 63.190 | −50.902 | 1.00 | 17.87 | A |
| ATOM | 634 | CA | THR | A | 85 | 9.201 | 61.969 | −51.072 | 1.00 | 18.31 | A |
| ATOM | 635 | CB | THR | A | 85 | 9.146 | 61.138 | −49.769 | 1.00 | 20.82 | A |
| ATOM | 636 | OG1 | THR | A | 85 | 8.742 | 61.977 | −48.680 | 1.00 | 28.34 | A |
| ATOM | 637 | CG2 | THR | A | 85 | 10.511 | 60.538 | −49.462 | 1.00 | 16.45 | A |
| ATOM | 638 | C | THR | A | 85 | 7.788 | 62.358 | −51.481 | 1.00 | 18.34 | A |
| ATOM | 639 | O | THR | A | 85 | 7.077 | 63.018 | −50.724 | 1.00 | 23.02 | A |
| ATOM | 640 | N | VAL | A | 86 | 7.395 | 61.976 | −52.693 | 1.00 | 15.85 | A |
| ATOM | 641 | CA | VAL | A | 86 | 6.067 | 62.287 | −53.194 | 1.00 | 16.53 | A |
| ATOM | 642 | CB | VAL | A | 86 | 6.110 | 62.679 | −54.693 | 1.00 | 18.47 | A |
| ATOM | 643 | CG1 | VAL | A | 86 | 4.713 | 63.033 | −55.180 | 1.00 | 18.91 | A |
| ATOM | 644 | CG2 | VAL | A | 86 | 7.035 | 63.871 | −54.893 | 1.00 | 11.80 | A |
| ATOM | 645 | C | VAL | A | 86 | 5.174 | 61.066 | −52.990 | 1.00 | 19.52 | A |
| ATOM | 646 | O | VAL | A | 86 | 5.441 | 59.982 | −53.515 | 1.00 | 17.30 | A |
| ATOM | 647 | N | LEU | A | 87 | 4.120 | 61.243 | −52.204 | 1.00 | 19.62 | A |
| ATOM | 648 | CA | LEU | A | 87 | 3.210 | 60.151 | −51.916 | 1.00 | 21.96 | A |
| ATOM | 649 | CB | LEU | A | 87 | 2.969 | 60.054 | −50.408 | 1.00 | 21.97 | A |
| ATOM | 650 | CG | LEU | A | 87 | 4.205 | 59.793 | −49.544 | 1.00 | 25.15 | A |
| ATOM | 651 | CD1 | LEU | A | 87 | 3.829 | 59.803 | −48.061 | 1.00 | 25.95 | A |
| ATOM | 652 | CD2 | LEU | A | 87 | 4.813 | 58.457 | −49.934 | 1.00 | 26.37 | A |
| ATOM | 653 | C | LEU | A | 87 | 1.880 | 60.310 | −52.626 | 1.00 | 23.08 | A |
| ATOM | 654 | O | LEU | A | 87 | 1.446 | 61.426 | −52.923 | 1.00 | 21.16 | A |
| ATOM | 655 | N | SER | A | 88 | 1.247 | 59.176 | −52.906 | 1.00 | 24.18 | A |
| ATOM | 656 | CA | SER | A | 88 | −0.059 | 59.153 | −53.551 | 1.00 | 27.67 | A |
| ATOM | 657 | CB | SER | A | 88 | −0.083 | 58.122 | −54.687 | 1.00 | 26.65 | A |
| ATOM | 658 | OG | SER | A | 88 | 0.290 | 58.731 | −55.909 | 1.00 | 27.52 | A |
| ATOM | 659 | C | SER | A | 88 | −1.070 | 58.784 | −52.473 | 1.00 | 27.07 | A |
| ATOM | 660 | O | SER | A | 88 | −1.581 | 57.662 | −52.431 | 1.00 | 28.13 | A |
| ATOM | 661 | N | GLU | A | 89 | −1.337 | 59.743 | −51.593 | 1.00 | 24.66 | A |
| ATOM | 662 | CA | GLU | A | 89 | −2.261 | 59.539 | −50.486 | 1.00 | 23.10 | A |
| ATOM | 663 | CB | GLU | A | 89 | −1.484 | 59.351 | −49.185 | 1.00 | 23.29 | A |
| ATOM | 664 | CG | GLU | A | 89 | −1.628 | 57.993 | −48.536 | 1.00 | 30.68 | A |
| ATOM | 665 | CD | GLU | A | 89 | −0.502 | 57.066 | −48.900 | 1.00 | 30.87 | A |
| ATOM | 666 | OE1 | GLU | A | 89 | 0.651 | 57.547 | −48.932 | 1.00 | 31.67 | A |
| ATOM | 667 | OE2 | GLU | A | 89 | −0.761 | 55.865 | −49.137 | 1.00 | 30.36 | A |
| ATOM | 668 | C | GLU | A | 89 | −3.188 | 60.733 | −50.319 | 1.00 | 19.89 | A |
| ATOM | 669 | O | GLU | A | 89 | −2.960 | 61.801 | −50.887 | 1.00 | 18.62 | A |
| ATOM | 670 | N | TRP | A | 90 | −4.236 | 60.544 | −49.529 | 1.00 | 15.67 | A |
| ATOM | 671 | CA | TRP | A | 90 | −5.178 | 61.611 | −49.263 | 1.00 | 16.08 | A |
| ATOM | 672 | CB | TRP | A | 90 | −6.516 | 61.051 | −48.793 | 1.00 | 15.10 | A |
| ATOM | 673 | CG | TRP | A | 90 | −7.451 | 60.669 | −49.885 | 1.00 | 17.07 | A |
| ATOM | 674 | CD2 | TRP | A | 90 | −8.352 | 61.540 | −50.568 | 1.00 | 15.35 | A |
| ATOM | 675 | CE2 | TRP | A | 90 | −9.067 | 60.755 | −51.501 | 1.00 | 17.21 | A |
| ATOM | 676 | CE3 | TRP | A | 90 | −8.625 | 62.909 | −50.484 | 1.00 | 16.70 | A |
| ATOM | 677 | CD1 | TRP | A | 90 | −7.643 | 59.422 | −50.414 | 1.00 | 15.95 | A |
| ATOM | 678 | NE1 | TRP | A | 90 | −8.616 | 59.467 | −51.387 | 1.00 | 17.51 | A |
| ATOM | 679 | CZ2 | TRP | A | 90 | −10.041 | 61.297 | −52.346 | 1.00 | 23.02 | A |
| ATOM | 680 | CZ3 | TRP | A | 90 | −9.595 | 63.450 | −51.325 | 1.00 | 22.97 | A |
| ATOM | 681 | CH2 | TRP | A | 90 | −10.291 | 62.643 | −52.244 | 1.00 | 23.23 | A |
| ATOM | 682 | C | TRP | A | 90 | −4.608 | 62.474 | −48.155 | 1.00 | 15.99 | A |
| ATOM | 683 | O | TRP | A | 90 | −4.805 | 63.691 | −48.133 | 1.00 | 16.97 | A |
| ATOM | 684 | N | LEU | A | 91 | −3.891 | 61.824 | −47.243 | 1.00 | 13.90 | A |
| ATOM | 685 | CA | LEU | A | 91 | −3.316 | 62.492 | −46.087 | 1.00 | 14.63 | A |
| ATOM | 686 | CB | LEU | A | 91 | −4.088 | 62.061 | −44.844 | 1.00 | 15.52 | A |
| ATOM | 687 | CG | LEU | A | 91 | −4.691 | 63.119 | −43.935 | 1.00 | 18.41 | A |
| ATOM | 688 | CD1 | LEU | A | 91 | −5.553 | 64.075 | −44.745 | 1.00 | 19.32 | A |
| ATOM | 689 | CD2 | LEU | A | 91 | −5.505 | 62.418 | −42.854 | 1.00 | 18.97 | A |
| ATOM | 690 | C | LEU | A | 91 | −1.837 | 62.195 | −45.861 | 1.00 | 12.15 | A |
| ATOM | 691 | O | LEU | A | 91 | −1.394 | 61.055 | −45.995 | 1.00 | 11.86 | A |
| ATOM | 692 | N | VAL | A | 92 | −1.081 | 63.223 | −45.495 | 1.00 | 10.61 | A |
| ATOM | 693 | CA | VAL | A | 92 | 0.336 | 63.049 | −45.205 | 1.00 | 10.75 | A |
| ATOM | 694 | CB | VAL | A | 92 | 1.221 | 63.468 | −46.405 | 1.00 | 10.93 | A |
| ATOM | 695 | CG1 | VAL | A | 92 | 1.024 | 64.951 | −46.719 | 1.00 | 8.77 | A |
| ATOM | 696 | CG2 | VAL | A | 92 | 2.682 | 63.170 | −46.094 | 1.00 | 7.61 | A |
| ATOM | 697 | C | VAL | A | 92 | 0.731 | 63.871 | −43.972 | 1.00 | 12.18 | A |
| ATOM | 698 | O | VAL | A | 92 | 0.317 | 65.020 | −43.820 | 1.00 | 15.59 | A |
| ATOM | 699 | N | LEU | A | 93 | 1.499 | 63.263 | −43.075 | 1.00 | 11.17 | A |
| ATOM | 700 | CA | LEU | A | 93 | 1.970 | 63.950 | −41.872 | 1.00 | 13.67 | A |
| ATOM | 701 | CB | LEU | A | 93 | 2.188 | 62.942 | −40.739 | 1.00 | 13.39 | A |
| ATOM | 702 | CG | LEU | A | 93 | 2.573 | 63.472 | −39.350 | 1.00 | 17.16 | A |
| ATOM | 703 | CD1 | LEU | A | 93 | 4.020 | 63.960 | −39.360 | 1.00 | 15.74 | A |
| ATOM | 704 | CD2 | LEU | A | 93 | 1.622 | 64.592 | −38.942 | 1.00 | 12.84 | A |
| ATOM | 705 | C | LEU | A | 93 | 3.297 | 64.590 | −42.278 | 1.00 | 11.61 | A |
| ATOM | 706 | O | LEU | A | 93 | 4.306 | 63.904 | −42.412 | 1.00 | 14.80 | A |
| ATOM | 707 | N | GLN | A | 94 | 3.299 | 65.901 | −42.473 | 1.00 | 8.42 | A |
| ATOM | 708 | CA | GLN | A | 94 | 4.506 | 66.584 | −42.928 | 1.00 | 12.60 | A |
| ATOM | 709 | CB | GLN | A | 94 | 4.114 | 67.751 | −43.849 | 1.00 | 10.69 | A |
| ATOM | 710 | CG | GLN | A | 94 | 3.258 | 67.325 | −45.045 | 1.00 | 12.02 | A |
| ATOM | 711 | CD | GLN | A | 94 | 3.136 | 68.400 | −46.117 | 1.00 | 13.46 | A |
| ATOM | 712 | OE1 | GLN | A | 94 | 2.524 | 69.444 | −45.904 | 1.00 | 7.99 | A |
| ATOM | 713 | NE2 | GLN | A | 94 | 3.727 | 68.141 | −47.282 | 1.00 | 13.54 | A |
| ATOM | 714 | C | GLN | A | 94 | 5.479 | 67.079 | −41.859 | 1.00 | 14.27 | A |
| ATOM | 715 | O | GLN | A | 94 | 5.080 | 67.508 | −40.775 | 1.00 | 14.76 | A |
| ATOM | 716 | N | THR | A | 95 | 6.767 | 67.016 | −42.190 | 1.00 | 13.46 | A |
| ATOM | 717 | CA | THR | A | 95 | 7.829 | 67.470 | −41.301 | 1.00 | 11.01 | A |
| ATOM | 718 | CB | THR | A | 95 | 8.378 | 66.306 | −40.447 | 1.00 | 13.23 | A |
| ATOM | 719 | OG1 | THR | A | 95 | 9.440 | 66.786 | −39.615 | 1.00 | 12.09 | A |
| ATOM | 720 | CG2 | THR | A | 95 | 8.897 | 65.175 | −41.333 | 1.00 | 11.57 | A |
| ATOM | 721 | C | THR | A | 95 | 8.975 | 68.064 | −42.128 | 1.00 | 12.90 | A |
| ATOM | 722 | O | THR | A | 95 | 9.274 | 67.584 | −43.226 | 1.00 | 13.73 | A |
| ATOM | 723 | N | PRO | A | 96 | 9.624 | 69.126 | −41.619 | 1.00 | 9.74 | A |
| ATOM | 724 | CD | PRO | A | 96 | 9.300 | 69.889 | −40.399 | 1.00 | 11.26 | A |
| ATOM | 725 | CA | PRO | A | 96 | 10.732 | 69.747 | −42.345 | 1.00 | 11.35 | A |
| ATOM | 726 | CB | PRO | A | 96 | 10.823 | 71.131 | −41.707 | 1.00 | 14.34 | A |
| ATOM | 727 | CG | PRO | A | 96 | 10.479 | 70.848 | −40.278 | 1.00 | 8.79 | A |
| ATOM | 728 | C | PRO | A | 96 | 12.024 | 68.939 | −42.193 | 1.00 | 14.00 | A |
| ATOM | 729 | O | PRO | A | 96 | 12.975 | 69.115 | −42.964 | 1.00 | 16.33 | A |
| ATOM | 730 | N | HIS | A | 97 | 12.050 | 68.055 | −41.196 | 1.00 | 12.09 | A |
| ATOM | 731 | CA | HIS | A | 97 | 13.212 | 67.206 | −40.936 | 1.00 | 12.06 | A |
| ATOM | 732 | CB | HIS | A | 97 | 14.216 | 67.932 | −40.037 | 1.00 | 10.92 | A |
| ATOM | 733 | CG | HIS | A | 97 | 14.783 | 69.177 | −40.639 | 1.00 | 15.58 | A |
| ATOM | 734 | CD2 | HIS | A | 97 | 14.540 | 70.484 | −40.380 | 1.00 | 17.58 | A |
| ATOM | 735 | ND1 | HIS | A | 97 | 15.712 | 69.154 | −41.657 | 1.00 | 15.37 | A |
| ATOM | 736 | CE1 | HIS | A | 97 | 16.018 | 70.393 | −41.997 | 1.00 | 15.73 | A |
| ATOM | 737 | NE2 | HIS | A | 97 | 15.321 | 71.218 | −41.238 | 1.00 | 18.56 | A |
| ATOM | 738 | C | HIS | A | 97 | 12.826 | 65.895 | −40.248 | 1.00 | 12.83 | A |
| ATOM | 739 | O | HIS | A | 97 | 11.785 | 65.804 | −39.588 | 1.00 | 13.44 | A |
| ATOM | 740 | N | LEU | A | 98 | 13.684 | 64.892 | −40.395 | 1.00 | 11.58 | A |
| ATOM | 741 | CA | LEU | A | 98 | 13.483 | 63.590 | −39.767 | 1.00 | 12.55 | A |
| ATOM | 742 | CB | LEU | A | 98 | 14.039 | 62.468 | −40.643 | 1.00 | 14.37 | A |
| ATOM | 743 | CG | LEU | A | 98 | 13.332 | 62.097 | −41.945 | 1.00 | 19.19 | A |
| ATOM | 744 | CD1 | LEU | A | 98 | 14.151 | 61.019 | −42.657 | 1.00 | 16.61 | A |
| ATOM | 745 | CD2 | LEU | A | 98 | 11.920 | 61.607 | −41.649 | 1.00 | 14.21 | A |
| ATOM | 746 | C | LEU | A | 98 | 14.254 | 63.589 | −38.457 | 1.00 | 15.41 | A |
| ATOM | 747 | O | LEU | A | 98 | 13.985 | 62.785 | −37.558 | 1.00 | 14.42 | A |
| ATOM | 748 | N | GLU | A | 99 | 15.223 | 64.496 | −38.371 | 1.00 | 15.30 | A |
| ATOM | 749 | CA | GLU | A | 99 | 16.085 | 64.626 | −37.205 | 1.00 | 18.38 | A |
| ATOM | 750 | CB | GLU | A | 99 | 17.516 | 64.242 | −37.598 | 1.00 | 23.63 | A |
| ATOM | 751 | CG | GLU | A | 99 | 18.600 | 64.506 | −36.552 | 1.00 | 35.76 | A |
| ATOM | 752 | CD | GLU | A | 99 | 18.822 | 63.337 | −35.603 | 1.00 | 42.72 | A |
| ATOM | 753 | OE1 | GLU | A | 99 | 18.853 | 62.178 | −36.076 | 1.00 | 46.27 | A |
| ATOM | 754 | OE2 | GLU | A | 99 | 18.984 | 63.579 | −34.385 | 1.00 | 46.86 | A |
| ATOM | 755 | C | GLU | A | 99 | 16.066 | 66.049 | −36.664 | 1.00 | 18.80 | A |
| ATOM | 756 | O | GLU | A | 99 | 16.188 | 67.011 | −37.421 | 1.00 | 21.42 | A |
| ATOM | 757 | N | PHE | A | 100 | 15.895 | 66.182 | −35.354 | 1.00 | 18.17 | A |
| ATOM | 758 | CA | PHE | A | 100 | 15.899 | 67.491 | −34.717 | 1.00 | 15.63 | A |
| ATOM | 759 | CB | PHE | A | 100 | 14.508 | 67.870 | −34.198 | 1.00 | 15.34 | A |
| ATOM | 760 | CG | PHE | A | 100 | 13.474 | 68.060 | −35.278 | 1.00 | 17.78 | A |
| ATOM | 761 | CD1 | PHE | A | 100 | 12.882 | 66.961 | −35.903 | 1.00 | 15.71 | A |
| ATOM | 762 | CD2 | PHE | A | 100 | 13.076 | 69.339 | −35.657 | 1.00 | 15.73 | A |
| ATOM | 763 | CE1 | PHE | A | 100 | 11.910 | 67.139 | −36.883 | 1.00 | 16.50 | A |
| ATOM | 764 | CE2 | PHE | A | 100 | 12.104 | 69.526 | −36.637 | 1.00 | 13.91 | A |
| ATOM | 765 | CZ | PHE | A | 100 | 11.521 | 68.425 | −37.250 | 1.00 | 13.57 | A |
| ATOM | 766 | C | PHE | A | 100 | 16.859 | 67.430 | −33.534 | 1.00 | 20.93 | A |
| ATOM | 767 | O | PHE | A | 100 | 17.114 | 66.351 | −32.996 | 1.00 | 19.46 | A |
| ATOM | 768 | N | GLN | A | 101 | 17.394 | 68.584 | −33.146 | 1.00 | 20.68 | A |
| ATOM | 769 | CA | GLN | A | 101 | 18.303 | 68.685 | −32.003 | 1.00 | 25.11 | A |
| ATOM | 770 | CB | GLN | A | 101 | 19.198 | 69.923 | −32.127 | 1.00 | 31.77 | A |
| ATOM | 771 | CG | GLN | A | 101 | 20.189 | 69.878 | −33.270 | 1.00 | 40.20 | A |
| ATOM | 772 | CD | GLN | A | 101 | 21.205 | 68.774 | −33.098 | 1.00 | 43.53 | A |
| ATOM | 773 | OE1 | GLN | A | 101 | 21.899 | 68.703 | −32.078 | 1.00 | 47.42 | A |
| ATOM | 774 | NE2 | GLN | A | 101 | 21.303 | 67.903 | −34.096 | 1.00 | 45.03 | A |
| ATOM | 775 | C | GLN | A | 101 | 17.448 | 68.837 | −30.754 | 1.00 | 21.87 | A |
| ATOM | 776 | O | GLN | A | 101 | 16.352 | 69.389 | −30.819 | 1.00 | 20.61 | A |
| ATOM | 777 | N | GLU | A | 102 | 17.937 | 68.365 | −29.616 | 1.00 | 19.70 | A |
| ATOM | 778 | CA | GLU | A | 102 | 17.160 | 68.501 | −28.394 | 1.00 | 21.63 | A |
| ATOM | 779 | CB | GLU | A | 102 | 17.839 | 67.765 | −27.242 | 1.00 | 25.91 | A |
| ATOM | 780 | CG | GLU | A | 102 | 17.007 | 67.763 | −25.973 | 1.00 | 36.76 | A |
| ATOM | 781 | CD | GLU | A | 102 | 17.353 | 66.622 | −25.041 | 1.00 | 41.14 | A |
| ATOM | 782 | OE1 | GLU | A | 102 | 18.553 | 66.436 | −24.735 | 1.00 | 45.54 | A |
| ATOM | 783 | OE2 | GLU | A | 102 | 16.418 | 65.914 | −24.610 | 1.00 | 44.05 | A |
| ATOM | 784 | C | GLU | A | 102 | 16.977 | 69.981 | −28.052 | 1.00 | 21.26 | A |
| ATOM | 785 | O | GLU | A | 102 | 17.917 | 70.779 | −28.149 | 1.00 | 20.71 | A |
| ATOM | 786 | N | GLY | A | 103 | 15.758 | 70.351 | −27.675 | 1.00 | 17.98 | A |
| ATOM | 787 | CA | GLY | A | 103 | 15.482 | 71.737 | −27.343 | 1.00 | 19.21 | A |
| ATOM | 788 | C | GLY | A | 103 | 14.955 | 72.517 | −28.532 | 1.00 | 18.31 | A |
| ATOM | 789 | O | GLY | A | 103 | 14.534 | 73.673 | −28.403 | 1.00 | 17.90 | A |
| ATOM | 790 | N | GLU | A | 104 | 14.970 | 71.874 | −29.696 | 1.00 | 18.09 | A |
| ATOM | 791 | CA | GLU | A | 104 | 14.491 | 72.488 | −30.930 | 1.00 | 16.83 | A |
| ATOM | 792 | CB | GLU | A | 104 | 15.188 | 71.820 | −32.112 | 1.00 | 20.19 | A |
| ATOM | 793 | CG | GLU | A | 104 | 14.991 | 72.498 | −33.447 | 1.00 | 25.11 | A |
| ATOM | 794 | CD | GLU | A | 104 | 15.869 | 71.890 | −34.522 | 1.00 | 28.89 | A |
| ATOM | 795 | OE1 | GLU | A | 104 | 15.820 | 72.370 | −35.679 | 1.00 | 31.83 | A |
| ATOM | 796 | OE2 | GLU | A | 104 | 16.609 | 70.933 | −34.207 | 1.00 | 23.18 | A |
| ATOM | 797 | C | GLU | A | 104 | 12.963 | 72.369 | −31.071 | 1.00 | 17.03 | A |
| ATOM | 798 | O | GLU | A | 104 | 12.328 | 71.500 | −30.464 | 1.00 | 14.61 | A |
| ATOM | 799 | N | THR | A | 105 | 12.372 | 73.258 | −31.862 | 1.00 | 15.12 | A |
| ATOM | 800 | CA | THR | A | 105 | 10.927 | 73.243 | −32.081 | 1.00 | 16.68 | A |
| ATOM | 801 | CB | THR | A | 105 | 10.368 | 74.655 | −32.370 | 1.00 | 18.31 | A |
| ATOM | 802 | OG1 | THR | A | 105 | 10.447 | 75.461 | −31.190 | 1.00 | 23.00 | A |
| ATOM | 803 | CG2 | THR | A | 105 | 8.914 | 74.567 | −32.801 | 1.00 | 23.09 | A |
| ATOM | 804 | C | THR | A | 105 | 10.549 | 72.365 | −33.265 | 1.00 | 16.18 | A |
| ATOM | 805 | O | THR | A | 105 | 11.089 | 72.515 | −34.359 | 1.00 | 15.58 | A |
| ATOM | 806 | N | ILE | A | 106 | 9.618 | 71.444 | −33.045 | 1.00 | 15.46 | A |
| ATOM | 807 | CA | ILE | A | 106 | 9.167 | 70.580 | −34.120 | 1.00 | 13.35 | A |
| ATOM | 808 | CB | ILE | A | 106 | 9.038 | 69.113 | −33.663 | 1.00 | 12.83 | A |
| ATOM | 809 | CG2 | ILE | A | 106 | 8.379 | 68.286 | −34.763 | 1.00 | 10.81 | A |
| ATOM | 810 | CG1 | ILE | A | 106 | 10.422 | 68.540 | −33.342 | 1.00 | 12.40 | A |
| ATOM | 811 | CD1 | ILE | A | 106 | 10.372 | 67.145 | −32.750 | 1.00 | 9.58 | A |
| ATOM | 812 | C | ILE | A | 106 | 7.807 | 71.059 | −34.608 | 1.00 | 13.62 | A |
| ATOM | 813 | O | ILE | A | 106 | 6.859 | 71.177 | −33.835 | 1.00 | 9.77 | A |
| ATOM | 814 | N | MET | A | 107 | 7.721 | 71.356 | −35.897 | 1.00 | 15.83 | A |
| ATOM | 815 | CA | MET | A | 107 | 6.460 | 71.789 | −36.474 | 1.00 | 15.67 | A |
| ATOM | 816 | CB | MET | A | 107 | 6.618 | 73.154 | −37.148 | 1.00 | 14.40 | A |
| ATOM | 817 | CG | MET | A | 107 | 6.858 | 74.291 | −36.163 | 1.00 | 21.53 | A |
| ATOM | 818 | SD | MET | A | 107 | 7.069 | 75.909 | −36.957 | 1.00 | 25.50 | A |
| ATOM | 819 | CE | MET | A | 107 | 5.371 | 76.360 | −37.256 | 1.00 | 23.60 | A |
| ATOM | 820 | C | MET | A | 107 | 6.003 | 70.741 | −37.478 | 1.00 | 13.71 | A |
| ATOM | 821 | O | MET | A | 107 | 6.751 | 70.358 | −38.386 | 1.00 | 7.12 | A |
| ATOM | 822 | N | LEU | A | 108 | 4.772 | 70.278 | −37.300 | 1.00 | 11.63 | A |
| ATOM | 823 | CA | LEU | A | 108 | 4.194 | 69.269 | −38.177 | 1.00 | 14.40 | A |
| ATOM | 824 | CB | LEU | A | 108 | 3.974 | 67.954 | −37.412 | 1.00 | 11.74 | A |
| ATOM | 825 | CG | LEU | A | 108 | 5.187 | 67.356 | −36.688 | 1.00 | 17.18 | A |
| ATOM | 826 | CD1 | LEU | A | 108 | 4.768 | 66.076 | −35.965 | 1.00 | 15.43 | A |
| ATOM | 827 | CD2 | LEU | A | 108 | 6.312 | 67.077 | −37.697 | 1.00 | 11.89 | A |
| ATOM | 828 | C | LEU | A | 108 | 2.863 | 69.740 | −38.734 | 1.00 | 14.47 | A |
| ATOM | 829 | O | LEU | A | 108 | 2.195 | 70.592 | −38.148 | 1.00 | 11.64 | A |
| ATOM | 830 | N | ARG | A | 109 | 2.485 | 69.177 | −39.875 | 1.00 | 14.05 | A |
| ATOM | 831 | CA | ARG | A | 109 | 1.223 | 69.506 | −40.506 | 1.00 | 14.99 | A |
| ATOM | 832 | CB | ARG | A | 109 | 1.414 | 70.503 | −41.658 | 1.00 | 14.00 | A |
| ATOM | 833 | CG | ARG | A | 109 | 0.090 | 70.908 | −42.306 | 1.00 | 16.17 | A |
| ATOM | 834 | CD | ARG | A | 109 | 0.204 | 72.064 | −43.304 | 1.00 | 15.17 | A |
| ATOM | 835 | NE | ARG | A | 109 | 0.819 | 71.670 | −44.570 | 1.00 | 19.63 | A |
| ATOM | 836 | CZ | ARG | A | 109 | 0.638 | 72.318 | −45.718 | 1.00 | 19.47 | A |
| ATOM | 837 | NH1 | ARG | A | 109 | 1.237 | 71.900 | −46.825 | 1.00 | 16.40 | A |
| ATOM | 838 | NH2 | ARG | A | 109 | −0.156 | 73.383 | −45.764 | 1.00 | 22.03 | A |
| ATOM | 839 | C | ARG | A | 109 | 0.578 | 68.245 | −41.050 | 1.00 | 14.10 | A |
| ATOM | 840 | O | ARG | A | 109 | 1.254 | 67.394 | −41.626 | 1.00 | 10.83 | A |
| ATOM | 841 | N | CYS | A | 110 | −0.723 | 68.108 | −40.829 | 1.00 | 14.44 | A |
| ATOM | 842 | CA | CYS | A | 110 | −1.457 | 66.970 | −41.365 | 1.00 | 15.09 | A |
| ATOM | 843 | C | CYS | A | 110 | −1.979 | 67.566 | −42.673 | 1.00 | 14.85 | A |
| ATOM | 844 | O | CYS | A | 110 | −2.884 | 68.397 | −42.657 | 1.00 | 14.18 | A |
| ATOM | 845 | CB | CYS | A | 110 | −2.619 | 66.591 | −40.443 | 1.00 | 15.85 | A |
| ATOM | 846 | SG | CYS | A | 110 | −3.447 | 65.020 | −40.871 | 1.00 | 17.44 | A |
| ATOM | 847 | N | HIS | A | 111 | −1.394 | 67.165 | −43.797 | 1.00 | 13.61 | A |
| ATOM | 848 | CA | HIS | A | 111 | −1.794 | 67.726 | −45.086 | 1.00 | 15.85 | A |
| ATOM | 849 | CB | HIS | A | 111 | −0.552 | 68.095 | −45.907 | 1.00 | 16.31 | A |
| ATOM | 850 | CG | HIS | A | 111 | −0.871 | 68.760 | −47.209 | 1.00 | 20.85 | A |
| ATOM | 851 | CD2 | HIS | A | 111 | −1.742 | 69.751 | −47.513 | 1.00 | 18.11 | A |
| ATOM | 852 | ND1 | HIS | A | 111 | −0.251 | 68.419 | −48.393 | 1.00 | 25.08 | A |
| ATOM | 853 | CE1 | HIS | A | 111 | −0.726 | 69.171 | −49.369 | 1.00 | 22.54 | A |
| ATOM | 854 | NE2 | HIS | A | 111 | −1.633 | 69.987 | −48.862 | 1.00 | 22.73 | A |
| ATOM | 855 | C | HIS | A | 111 | −2.708 | 66.859 | −45.946 | 1.00 | 12.53 | A |
| ATOM | 856 | O | HIS | A | 111 | −2.415 | 65.695 | −46.200 | 1.00 | 13.13 | A |
| ATOM | 857 | N | SER | A | 112 | −3.810 | 67.448 | −46.400 | 1.00 | 10.79 | A |
| ATOM | 858 | CA | SER | A | 112 | −4.761 | 66.740 | −47.249 | 1.00 | 16.48 | A |
| ATOM | 859 | CB | SER | A | 112 | −6.188 | 67.205 | −46.944 | 1.00 | 15.74 | A |
| ATOM | 860 | OG | SER | A | 112 | −6.331 | 68.597 | −47.174 | 1.00 | 17.69 | A |
| ATOM | 861 | C | SER | A | 112 | −4.436 | 66.996 | −48.729 | 1.00 | 17.59 | A |
| ATOM | 862 | O | SER | A | 112 | −3.835 | 68.014 | −49.085 | 1.00 | 16.95 | A |
| ATOM | 863 | N | TRP | A | 113 | −4.840 | 66.072 | −49.589 | 1.00 | 17.22 | A |
| ATOM | 864 | CA | TRP | A | 113 | −4.589 | 66.205 | −51.017 | 1.00 | 18.24 | A |
| ATOM | 865 | CB | TRP | A | 113 | −5.031 | 64.930 | −51.723 | 1.00 | 18.21 | A |
| ATOM | 866 | CG | TRP | A | 113 | −4.816 | 64.936 | −53.201 | 1.00 | 26.27 | A |
| ATOM | 867 | CD2 | TRP | A | 113 | −5.831 | 64.874 | −54.210 | 1.00 | 28.30 | A |
| ATOM | 868 | CE2 | TRP | A | 113 | −5.176 | 64.852 | −55.461 | 1.00 | 28.85 | A |
| ATOM | 869 | CE3 | TRP | A | 113 | −7.231 | 64.832 | −54.177 | 1.00 | 31.03 | A |
| ATOM | 870 | CD1 | TRP | A | 113 | −3.620 | 64.955 | −53.859 | 1.00 | 25.77 | A |
| ATOM | 871 | NE1 | TRP | A | 113 | −3.828 | 64.903 | −55.220 | 1.00 | 29.20 | A |
| ATOM | 872 | CZ2 | TRP | A | 113 | −5.875 | 64.788 | −56.672 | 1.00 | 27.26 | A |
| ATOM | 873 | CZ3 | TRP | A | 113 | −7.926 | 64.768 | −55.383 | 1.00 | 31.90 | A |
| ATOM | 874 | CH2 | TRP | A | 113 | −7.244 | 64.746 | −56.613 | 1.00 | 30.26 | A |
| ATOM | 875 | C | TRP | A | 113 | −5.304 | 67.423 | −51.620 | 1.00 | 19.18 | A |
| ATOM | 876 | O | TRP | A | 113 | −6.496 | 67.644 | −51.388 | 1.00 | 17.92 | A |
| ATOM | 877 | N | LYS | A | 114 | −4.561 | 68.211 | −52.391 | 1.00 | 19.66 | A |
| ATOM | 878 | CA | LYS | A | 114 | −5.098 | 69.408 | −53.032 | 1.00 | 23.95 | A |
| ATOM | 879 | CB | LYS | A | 114 | −6.195 | 69.035 | −54.034 | 1.00 | 26.44 | A |
| ATOM | 880 | CG | LYS | A | 114 | −5.712 | 68.138 | −55.164 | 1.00 | 33.00 | A |
| ATOM | 881 | CD | LYS | A | 114 | −6.344 | 68.509 | −56.499 | 1.00 | 36.46 | A |
| ATOM | 882 | CE | LYS | A | 114 | −5.874 | 69.883 | −56.962 | 1.00 | 39.24 | A |
| ATOM | 883 | NZ | LYS | A | 114 | −6.301 | 70.186 | −58.357 | 1.00 | 43.68 | A |
| ATOM | 884 | C | LYS | A | 114 | −5.646 | 70.417 | −52.027 | 1.00 | 24.48 | A |
| ATOM | 885 | O | LYS | A | 114 | −6.450 | 71.282 | −52.374 | 1.00 | 23.07 | A |
| ATOM | 886 | N | ASP | A | 115 | −5.199 | 70.303 | −50.781 | 1.00 | 26.44 | A |
| ATOM | 887 | CA | ASP | A | 115 | −5.627 | 71.203 | −49.719 | 1.00 | 25.64 | A |
| ATOM | 888 | CB | ASP | A | 115 | −5.146 | 72.626 | −50.000 | 1.00 | 24.47 | A |
| ATOM | 889 | CG | ASP | A | 115 | −3.657 | 72.785 | −49.770 | 1.00 | 27.91 | A |
| ATOM | 890 | OD1 | ASP | A | 115 | −3.204 | 72.509 | −48.640 | 1.00 | 27.91 | A |
| ATOM | 891 | OD2 | ASP | A | 115 | −2.939 | 73.176 | −50.711 | 1.00 | 25.83 | A |
| ATOM | 892 | C | ASP | A | 115 | −7.126 | 71.211 | −49.478 | 1.00 | 26.09 | A |
| ATOM | 893 | O | ASP | A | 115 | −7.702 | 72.261 | −49.186 | 1.00 | 26.70 | A |
| ATOM | 894 | N | LYS | A | 116 | −7.756 | 70.047 | −49.610 | 1.00 | 22.56 | A |
| ATOM | 895 | CA | LYS | A | 116 | −9.184 | 69.941 | −49.357 | 1.00 | 24.65 | A |
| ATOM | 896 | CB | LYS | A | 116 | −9.711 | 68.563 | −49.773 | 1.00 | 24.20 | A |
| ATOM | 897 | CG | LYS | A | 116 | −9.837 | 68.377 | −51.274 | 1.00 | 25.09 | A |
| ATOM | 898 | CD | LYS | A | 116 | −10.185 | 66.946 | −51.628 | 1.00 | 28.57 | A |
| ATOM | 899 | CE | LYS | A | 116 | −10.376 | 66.789 | −53.130 | 1.00 | 31.31 | A |
| ATOM | 900 | NZ | LYS | A | 116 | −11.532 | 67.584 | −53.633 | 1.00 | 30.73 | A |
| ATOM | 901 | C | LYS | A | 116 | −9.384 | 70.142 | −47.858 | 1.00 | 25.28 | A |
| ATOM | 902 | O | LYS | A | 116 | −8.599 | 69.647 | −47.047 | 1.00 | 22.83 | A |
| ATOM | 903 | N | PRO | A | 117 | −10.431 | 70.889 | −47.473 | 1.00 | 27.81 | A |
| ATOM | 904 | CD | PRO | A | 117 | −11.421 | 71.536 | −48.356 | 1.00 | 30.50 | A |
| ATOM | 905 | CA | PRO | A | 117 | −10.735 | 71.161 | −46.064 | 1.00 | 27.35 | A |
| ATOM | 906 | CB | PRO | A | 117 | −12.154 | 71.714 | −46.132 | 1.00 | 28.79 | A |
| ATOM | 907 | CG | PRO | A | 117 | −12.116 | 72.505 | −47.415 | 1.00 | 29.10 | A |
| ATOM | 908 | C | PRO | A | 117 | −10.615 | 69.931 | −45.156 | 1.00 | 24.99 | A |
| ATOM | 909 | O | PRO | A | 117 | −11.239 | 68.897 | −45.395 | 1.00 | 22.26 | A |
| ATOM | 910 | N | LEU | A | 118 | −9.801 | 70.064 | −44.114 | 1.00 | 22.83 | A |
| ATOM | 911 | CA | LEU | A | 118 | −9.564 | 68.986 | −43.161 | 1.00 | 21.31 | A |
| ATOM | 912 | CB | LEU | A | 118 | −8.062 | 68.680 | −43.124 | 1.00 | 21.60 | A |
| ATOM | 913 | CG | LEU | A | 118 | −7.514 | 67.500 | −42.314 | 1.00 | 22.97 | A |
| ATOM | 914 | CD1 | LEU | A | 118 | −8.077 | 66.172 | −42.838 | 1.00 | 19.52 | A |
| ATOM | 915 | CD2 | LEU | A | 118 | −5.995 | 67.516 | −42.404 | 1.00 | 15.85 | A |
| ATOM | 916 | C | LEU | A | 118 | −10.050 | 69.441 | −41.788 | 1.00 | 19.25 | A |
| ATOM | 917 | O | LEU | A | 118 | −9.759 | 70.561 | −41.371 | 1.00 | 20.52 | A |
| ATOM | 918 | N | VAL | A | 119 | −10.798 | 68.586 | −41.094 | 1.00 | 19.63 | A |
| ATOM | 919 | CA | VAL | A | 119 | −11.322 | 68.920 | −39.763 | 1.00 | 18.77 | A |
| ATOM | 920 | CB | VAL | A | 119 | −12.798 | 69.380 | −39.822 | 1.00 | 18.33 | A |
| ATOM | 921 | CG1 | VAL | A | 119 | −12.932 | 70.605 | −40.713 | 1.00 | 20.19 | A |
| ATOM | 922 | CG2 | VAL | A | 119 | −13.676 | 68.247 | −40.315 | 1.00 | 16.12 | A |
| ATOM | 923 | C | VAL | A | 119 | −11.251 | 67.742 | −38.799 | 1.00 | 18.39 | A |
| ATOM | 924 | O | VAL | A | 119 | −11.196 | 66.587 | −39.224 | 1.00 | 19.05 | A |
| ATOM | 925 | N | LYS | A | 120 | −11.268 | 68.043 | −37.500 | 1.00 | 15.53 | A |
| ATOM | 926 | CA | LYS | A | 120 | −11.204 | 67.018 | −36.459 | 1.00 | 12.90 | A |
| ATOM | 927 | CB | LYS | A | 120 | −12.449 | 66.130 | −36.527 | 1.00 | 12.34 | A |
| ATOM | 928 | CG | LYS | A | 120 | −13.745 | 66.917 | −36.329 | 1.00 | 17.22 | A |
| ATOM | 929 | CD | LYS | A | 120 | −14.984 | 66.034 | −36.394 | 1.00 | 20.25 | A |
| ATOM | 930 | CE | LYS | A | 120 | −16.259 | 66.883 | −36.397 | 1.00 | 24.61 | A |
| ATOM | 931 | NZ | LYS | A | 120 | −17.510 | 66.060 | −36.366 | 1.00 | 25.37 | A |
| ATOM | 932 | C | LYS | A | 120 | −9.935 | 66.199 | −36.642 | 1.00 | 11.27 | A |
| ATOM | 933 | O | LYS | A | 120 | −9.961 | 64.974 | −36.785 | 1.00 | 12.71 | A |
| ATOM | 934 | N | VAL | A | 121 | −8.817 | 66.910 | −36.632 | 1.00 | 14.00 | A |
| ATOM | 935 | CA | VAL | A | 121 | −7.496 | 66.326 | −36.823 | 1.00 | 11.51 | A |
| ATOM | 936 | CB | VAL | A | 121 | −6.550 | 67.359 | −37.486 | 1.00 | 9.69 | A |
| ATOM | 937 | CG1 | VAL | A | 121 | −5.153 | 66.780 | −37.626 | 1.00 | 6.50 | A |
| ATOM | 938 | CG2 | VAL | A | 121 | −7.107 | 67.774 | −38.857 | 1.00 | 8.78 | A |
| ATOM | 939 | C | VAL | A | 121 | −6.842 | 65.837 | −35.535 | 1.00 | 12.34 | A |
| ATOM | 940 | O | VAL | A | 121 | −6.824 | 66.545 | −34.529 | 1.00 | 9.85 | A |
| ATOM | 941 | N | THR | A | 122 | −6.294 | 64.624 | −35.584 | 1.00 | 12.28 | A |
| ATOM | 942 | CA | THR | A | 122 | −5.608 | 64.046 | −34.438 | 1.00 | 11.07 | A |
| ATOM | 943 | CB | THR | A | 122 | −6.274 | 62.721 | −33.959 | 1.00 | 13.43 | A |
| ATOM | 944 | OG1 | THR | A | 122 | −7.693 | 62.891 | −33.831 | 1.00 | 16.38 | A |
| ATOM | 945 | CG2 | THR | A | 122 | −5.705 | 62.304 | −32.620 | 1.00 | 7.72 | A |
| ATOM | 946 | C | THR | A | 122 | −4.172 | 63.705 | −34.840 | 1.00 | 11.90 | A |
| ATOM | 947 | O | THR | A | 122 | −3.950 | 63.055 | −35.858 | 1.00 | 11.67 | A |
| ATOM | 948 | N | PHE | A | 123 | −3.199 | 64.149 | −34.050 | 1.00 | 12.53 | A |
| ATOM | 949 | CA | PHE | A | 123 | −1.799 | 63.824 | −34.316 | 1.00 | 14.96 | A |
| ATOM | 950 | CB | PHE | A | 123 | −0.900 | 65.050 | −34.103 | 1.00 | 11.60 | A |
| ATOM | 951 | CG | PHE | A | 123 | −1.066 | 66.121 | −35.144 | 1.00 | 14.65 | A |
| ATOM | 952 | CD1 | PHE | A | 123 | −2.077 | 67.072 | −35.033 | 1.00 | 12.53 | A |
| ATOM | 953 | CD2 | PHE | A | 123 | −0.204 | 66.181 | −36.237 | 1.00 | 14.43 | A |
| ATOM | 954 | CE1 | PHE | A | 123 | −2.229 | 68.070 | −35.991 | 1.00 | 14.25 | A |
| ATOM | 955 | CE2 | PHE | A | 123 | −0.345 | 67.175 | −37.201 | 1.00 | 19.79 | A |
| ATOM | 956 | CZ | PHE | A | 123 | −1.362 | 68.124 | −37.076 | 1.00 | 16.58 | A |
| ATOM | 957 | C | PHE | A | 123 | −1.373 | 62.711 | −33.345 | 1.00 | 15.11 | A |
| ATOM | 958 | O | PHE | A | 123 | −1.662 | 62.794 | −32.154 | 1.00 | 19.69 | A |
| ATOM | 959 | N | PHE | A | 124 | −0.692 | 61.677 | −33.840 | 1.00 | 14.48 | A |
| ATOM | 960 | CA | PHE | A | 124 | −0.251 | 60.578 | −32.969 | 1.00 | 12.17 | A |
| ATOM | 961 | CB | PHE | A | 124 | −0.813 | 59.220 | −33.418 | 1.00 | 11.56 | A |
| ATOM | 962 | CG | PHE | A | 124 | −2.319 | 59.142 | −33.501 | 1.00 | 12.90 | A |
| ATOM | 963 | CD1 | PHE | A | 124 | −3.006 | 59.711 | −34.571 | 1.00 | 10.13 | A |
| ATOM | 964 | CD2 | PHE | A | 124 | −3.043 | 58.435 | −32.541 | 1.00 | 10.53 | A |
| ATOM | 965 | CE1 | PHE | A | 124 | −4.388 | 59.574 | −34.689 | 1.00 | 10.22 | A |
| ATOM | 966 | CE2 | PHE | A | 124 | −4.428 | 58.289 | −32.646 | 1.00 | 9.47 | A |
| ATOM | 967 | CZ | PHE | A | 124 | −5.102 | 58.858 | −33.721 | 1.00 | 13.36 | A |
| ATOM | 968 | C | PHE | A | 124 | 1.263 | 60.407 | −32.938 | 1.00 | 13.00 | A |
| ATOM | 969 | O | PHE | A | 124 | 1.951 | 60.656 | −33.928 | 1.00 | 11.00 | A |
| ATOM | 970 | N | GLN | A | 125 | 1.762 | 59.945 | −31.793 | 1.00 | 14.02 | A |
| ATOM | 971 | CA | GLN | A | 125 | 3.177 | 59.652 | −31.599 | 1.00 | 11.92 | A |
| ATOM | 972 | CB | GLN | A | 125 | 3.799 | 60.550 | −30.526 | 1.00 | 11.13 | A |
| ATOM | 973 | CG | GLN | A | 125 | 5.331 | 60.441 | −30.471 | 1.00 | 13.73 | A |
| ATOM | 974 | CD | GLN | A | 125 | 5.918 | 60.836 | −29.125 | 1.00 | 16.37 | A |
| ATOM | 975 | OE1 | GLN | A | 125 | 5.365 | 61.683 | −28.417 | 1.00 | 13.84 | A |
| ATOM | 976 | NE2 | GLN | A | 125 | 7.056 | 60.232 | −28.771 | 1.00 | 10.34 | A |
| ATOM | 977 | C | GLN | A | 125 | 3.184 | 58.202 | −31.112 | 1.00 | 10.94 | A |
| ATOM | 978 | O | GLN | A | 125 | 2.651 | 57.905 | −30.045 | 1.00 | 15.94 | A |
| ATOM | 979 | N | ASN | A | 126 | 3.774 | 57.302 | −31.889 | 1.00 | 12.52 | A |
| ATOM | 980 | CA | ASN | A | 126 | 3.804 | 55.881 | −31.529 | 1.00 | 14.10 | A |
| ATOM | 981 | CB | ASN | A | 126 | 4.726 | 55.632 | −30.332 | 1.00 | 10.14 | A |
| ATOM | 982 | CG | ASN | A | 126 | 6.167 | 55.943 | −30.639 | 1.00 | 10.60 | A |
| ATOM | 983 | OD1 | ASN | A | 126 | 6.651 | 55.659 | −31.733 | 1.00 | 14.02 | A |
| ATOM | 984 | ND2 | ASN | A | 126 | 6.869 | 56.523 | −29.672 | 1.00 | 12.27 | A |
| ATOM | 985 | C | ASN | A | 126 | 2.413 | 55.334 | −31.203 | 1.00 | 13.15 | A |
| ATOM | 986 | O | ASN | A | 126 | 2.266 | 54.474 | −30.334 | 1.00 | 15.57 | A |
| ATOM | 987 | N | GLY | A | 127 | 1.395 | 55.848 | −31.888 | 1.00 | 13.11 | A |
| ATOM | 988 | CA | GLY | A | 127 | 0.039 | 55.371 | −31.665 | 1.00 | 10.62 | A |
| ATOM | 989 | C | GLY | A | 127 | −0.767 | 56.113 | −30.615 | 1.00 | 11.54 | A |
| ATOM | 990 | O | GLY | A | 127 | −1.979 | 55.920 | −30.525 | 1.00 | 10.34 | A |
| ATOM | 991 | N | LYS | A | 128 | −0.106 | 56.956 | −29.825 | 1.00 | 11.68 | A |
| ATOM | 992 | CA | LYS | A | 128 | −0.777 | 57.719 | −28.774 | 1.00 | 15.74 | A |
| ATOM | 993 | CB | LYS | A | 128 | 0.098 | 57.803 | −27.511 | 1.00 | 11.67 | A |
| ATOM | 994 | CG | LYS | A | 128 | 0.305 | 56.485 | −26.775 | 1.00 | 14.35 | A |
| ATOM | 995 | CD | LYS | A | 128 | 1.204 | 56.687 | −25.565 | 1.00 | 15.99 | A |
| ATOM | 996 | CE | LYS | A | 128 | 1.262 | 55.439 | −24.688 | 1.00 | 18.25 | A |
| ATOM | 997 | NZ | LYS | A | 128 | −0.042 | 55.180 | −24.015 | 1.00 | 22.23 | A |
| ATOM | 998 | C | LYS | A | 128 | −1.109 | 59.138 | −29.219 | 1.00 | 16.21 | A |
| ATOM | 999 | O | LYS | A | 128 | −0.232 | 59.871 | −29.680 | 1.00 | 14.98 | A |
| ATOM | 1000 | N | SER | A | 129 | −2.371 | 59.524 | −29.054 | 1.00 | 16.12 | A |
| ATOM | 1001 | CA | SER | A | 129 | −2.815 | 60.862 | −29.421 | 1.00 | 19.68 | A |
| ATOM | 1002 | CB | SER | A | 129 | −4.324 | 61.005 | −29.231 | 1.00 | 17.34 | A |
| ATOM | 1003 | OG | SER | A | 129 | −4.723 | 62.330 | −29.539 | 1.00 | 22.40 | A |
| ATOM | 1004 | C | SER | A | 129 | −2.112 | 61.919 | −28.579 | 1.00 | 21.10 | A |
| ATOM | 1005 | O | SER | A | 129 | −2.135 | 61.869 | −27.347 | 1.00 | 20.39 | A |
| ATOM | 1006 | N | GLN | A | 130 | −1.498 | 62.880 | −29.254 | 1.00 | 20.36 | A |
| ATOM | 1007 | CA | GLN | A | 130 | −0.789 | 63.947 | −28.578 | 1.00 | 22.55 | A |
| ATOM | 1008 | CB | GLN | A | 130 | 0.566 | 64.177 | −29.245 | 1.00 | 22.08 | A |
| ATOM | 1009 | CG | GLN | A | 130 | 1.496 | 62.982 | −29.203 | 1.00 | 19.22 | A |
| ATOM | 1010 | CD | GLN | A | 130 | 1.847 | 62.567 | −27.790 | 1.00 | 19.25 | A |
| ATOM | 1011 | OE1 | GLN | A | 130 | 2.309 | 63.383 | −26.988 | 1.00 | 16.42 | A |
| ATOM | 1012 | NE2 | GLN | A | 130 | 1.639 | 61.289 | −27.477 | 1.00 | 15.96 | A |
| ATOM | 1013 | C | GLN | A | 130 | −1.605 | 65.232 | −28.626 | 1.00 | 24.17 | A |
| ATOM | 1014 | O | GLN | A | 130 | −1.416 | 66.131 | −27.804 | 1.00 | 24.77 | A |
| ATOM | 1015 | N | LYS | A | 131 | −2.514 | 65.314 | −29.591 | 1.00 | 22.40 | A |
| ATOM | 1016 | CA | LYS | A | 131 | −3.336 | 66.503 | −29.741 | 1.00 | 21.44 | A |
| ATOM | 1017 | CB | LYS | A | 131 | −2.481 | 67.671 | −30.256 | 1.00 | 22.38 | A |
| ATOM | 1018 | CG | LYS | A | 131 | −3.302 | 68.869 | −30.725 | 1.00 | 23.95 | A |
| ATOM | 1019 | CD | LYS | A | 131 | −2.448 | 69.952 | −31.366 | 1.00 | 26.96 | A |
| ATOM | 1020 | CE | LYS | A | 131 | −3.330 | 71.027 | −32.000 | 1.00 | 27.92 | A |
| ATOM | 1021 | NZ | LYS | A | 131 | −2.567 | 71.987 | −32.852 | 1.00 | 26.37 | A |
| ATOM | 1022 | C | LYS | A | 131 | −4.515 | 66.301 | −30.685 | 1.00 | 19.30 | A |
| ATOM | 1023 | O | LYS | A | 131 | −4.412 | 65.602 | −31.693 | 1.00 | 19.51 | A |
| ATOM | 1024 | N | PHE | A | 132 | −5.637 | 66.923 | −30.343 | 1.00 | 18.99 | A |
| ATOM | 1025 | CA | PHE | A | 132 | −6.836 | 66.862 | −31.165 | 1.00 | 17.71 | A |
| ATOM | 1026 | CB | PHE | A | 132 | −7.932 | 66.005 | −30.533 | 1.00 | 12.30 | A |
| ATOM | 1027 | CG | PHE | A | 132 | −9.253 | 66.103 | −31.265 | 1.00 | 19.05 | A |
| ATOM | 1028 | CD1 | PHE | A | 132 | −9.506 | 65.315 | −32.387 | 1.00 | 15.10 | A |
| ATOM | 1029 | CD2 | PHE | A | 132 | −10.207 | 67.048 | −30.885 | 1.00 | 15.71 | A |
| ATOM | 1030 | CE1 | PHE | A | 132 | −10.684 | 65.470 | −33.123 | 1.00 | 16.69 | A |
| ATOM | 1031 | CE2 | PHE | A | 132 | −11.386 | 67.212 | −31.613 | 1.00 | 15.07 | A |
| ATOM | 1032 | CZ | PHE | A | 132 | −11.625 | 66.421 | −32.737 | 1.00 | 13.16 | A |
| ATOM | 1033 | C | PHE | A | 132 | −7.387 | 68.266 | −31.312 | 1.00 | 19.25 | A |
| ATOM | 1034 | O | PHE | A | 132 | −7.538 | 68.981 | −30.322 | 1.00 | 20.96 | A |
| ATOM | 1035 | N | SER | A | 133 | −7.710 | 68.655 | −32.539 | 1.00 | 19.74 | A |
| ATOM | 1036 | CA | SER | A | 133 | −8.274 | 69.978 | −32.777 | 1.00 | 20.43 | A |
| ATOM | 1037 | CB | SER | A | 133 | −7.178 | 70.957 | −33.207 | 1.00 | 19.82 | A |
| ATOM | 1038 | OG | SER | A | 133 | −7.732 | 72.081 | −33.861 | 1.00 | 17.06 | A |
| ATOM | 1039 | C | SER | A | 133 | −9.351 | 69.908 | −33.848 | 1.00 | 20.20 | A |
| ATOM | 1040 | O | SER | A | 133 | −9.193 | 69.211 | −34.854 | 1.00 | 22.15 | A |
| ATOM | 1041 | N | ARG | A | 134 | −10.446 | 70.628 | −33.633 | 1.00 | 18.23 | A |
| ATOM | 1042 | CA | ARG | A | 134 | −11.534 | 70.643 | −34.602 | 1.00 | 20.44 | A |
| ATOM | 1043 | CB | ARG | A | 134 | −12.759 | 71.365 | −34.033 | 1.00 | 19.88 | A |
| ATOM | 1044 | CG | ARG | A | 134 | −13.610 | 70.534 | −33.088 | 1.00 | 24.62 | A |
| ATOM | 1045 | CD | ARG | A | 134 | −14.730 | 71.382 | −32.480 | 1.00 | 27.17 | A |
| ATOM | 1046 | NE | ARG | A | 134 | −15.778 | 71.757 | −33.434 | 1.00 | 25.50 | A |
| ATOM | 1047 | CZ | ARG | A | 134 | −16.741 | 70.940 | −33.857 | 1.00 | 26.60 | A |
| ATOM | 1048 | NH1 | ARG | A | 134 | −17.650 | 71.374 | −34.720 | 1.00 | 24.47 | A |
| ATOM | 1049 | NH2 | ARG | A | 134 | −16.800 | 69.688 | −33.418 | 1.00 | 28.75 | A |
| ATOM | 1050 | C | ARG | A | 134 | −11.154 | 71.301 | −35.924 | 1.00 | 19.61 | A |
| ATOM | 1051 | O | ARG | A | 134 | −11.629 | 70.883 | −36.975 | 1.00 | 18.29 | A |
| ATOM | 1052 | N | LEU | A | 135 | −10.294 | 72.316 | −35.874 | 1.00 | 21.59 | A |
| ATOM | 1053 | CA | LEU | A | 135 | −9.905 | 73.041 | −37.083 | 1.00 | 22.38 | A |
| ATOM | 1054 | CB | LEU | A | 135 | −10.581 | 74.418 | −37.087 | 1.00 | 24.69 | A |
| ATOM | 1055 | CG | LEU | A | 135 | −12.111 | 74.464 | −37.004 | 1.00 | 26.93 | A |
| ATOM | 1056 | CD1 | LEU | A | 135 | −12.576 | 75.899 | −36.792 | 1.00 | 24.73 | A |
| ATOM | 1057 | CD2 | LEU | A | 135 | −12.706 | 73.883 | −38.279 | 1.00 | 25.69 | A |
| ATOM | 1058 | C | LEU | A | 135 | −8.411 | 73.252 | −37.328 | 1.00 | 24.36 | A |
| ATOM | 1059 | O | LEU | A | 135 | −8.027 | 73.710 | −38.403 | 1.00 | 24.81 | A |
| ATOM | 1060 | N | ASP | A | 136 | −7.562 | 72.940 | −36.357 | 1.00 | 23.03 | A |
| ATOM | 1061 | CA | ASP | A | 136 | −6.132 | 73.166 | −36.552 | 1.00 | 24.39 | A |
| ATOM | 1062 | CB | ASP | A | 136 | −5.518 | 73.717 | −35.256 | 1.00 | 25.93 | A |
| ATOM | 1063 | CG | ASP | A | 136 | −4.054 | 74.096 | −35.410 | 1.00 | 26.74 | A |
| ATOM | 1064 | OD1 | ASP | A | 136 | −3.623 | 74.415 | −36.539 | 1.00 | 25.88 | A |
| ATOM | 1065 | OD2 | ASP | A | 136 | −3.334 | 74.091 | −34.389 | 1.00 | 27.65 | A |
| ATOM | 1066 | C | ASP | A | 136 | −5.365 | 71.930 | −37.032 | 1.00 | 23.84 | A |
| ATOM | 1067 | O | ASP | A | 136 | −5.147 | 70.979 | −36.274 | 1.00 | 25.39 | A |
| ATOM | 1068 | N | PRO | A | 137 | −4.947 | 71.933 | −38.312 | 1.00 | 20.68 | A |
| ATOM | 1069 | CD | PRO | A | 137 | −5.273 | 72.964 | −39.316 | 1.00 | 18.17 | A |
| ATOM | 1070 | CA | PRO | A | 137 | −4.199 | 70.833 | −38.936 | 1.00 | 17.35 | A |
| ATOM | 1071 | CB | PRO | A | 137 | −4.520 | 71.013 | −40.410 | 1.00 | 15.18 | A |
| ATOM | 1072 | CG | PRO | A | 137 | −4.472 | 72.502 | −40.536 | 1.00 | 17.24 | A |
| ATOM | 1073 | C | PRO | A | 137 | −2.699 | 70.926 | −38.677 | 1.00 | 14.74 | A |
| ATOM | 1074 | O | PRO | A | 137 | −1.905 | 70.233 | −39.318 | 1.00 | 14.32 | A |
| ATOM | 1075 | N | THR | A | 138 | −2.310 | 71.803 | −37.757 | 1.00 | 15.92 | A |
| ATOM | 1076 | CA | THR | A | 138 | −0.899 | 71.975 | −37.436 | 1.00 | 17.43 | A |
| ATOM | 1077 | CB | THR | A | 138 | −0.454 | 73.441 | −37.581 | 1.00 | 16.61 | A |
| ATOM | 1078 | OG1 | THR | A | 138 | −1.074 | 74.226 | −36.555 | 1.00 | 20.98 | A |
| ATOM | 1079 | CG2 | THR | A | 138 | −0.848 | 73.986 | −38.949 | 1.00 | 9.13 | A |
| ATOM | 1080 | C | THR | A | 138 | −0.619 | 71.532 | −36.010 | 1.00 | 18.18 | A |
| ATOM | 1081 | O | THR | A | 138 | −1.498 | 71.569 | −35.146 | 1.00 | 18.15 | A |
| ATOM | 1082 | N | PHE | A | 139 | 0.623 | 71.133 | −35.769 | 1.00 | 17.53 | A |
| ATOM | 1083 | CA | PHE | A | 139 | 1.039 | 70.660 | −34.464 | 1.00 | 16.58 | A |
| ATOM | 1084 | CB | PHE | A | 139 | 0.911 | 69.130 | −34.435 | 1.00 | 16.50 | A |
| ATOM | 1085 | CG | PHE | A | 139 | 1.269 | 68.506 | −33.121 | 1.00 | 15.47 | A |
| ATOM | 1086 | CD1 | PHE | A | 139 | 0.777 | 69.024 | −31.929 | 1.00 | 16.19 | A |
| ATOM | 1087 | CD2 | PHE | A | 139 | 2.078 | 67.375 | −33.079 | 1.00 | 15.06 | A |
| ATOM | 1088 | CE1 | PHE | A | 139 | 1.086 | 68.421 | −30.708 | 1.00 | 15.97 | A |
| ATOM | 1089 | CE2 | PHE | A | 139 | 2.391 | 66.767 | −31.865 | 1.00 | 14.72 | A |
| ATOM | 1090 | CZ | PHE | A | 139 | 1.893 | 67.291 | −30.680 | 1.00 | 15.09 | A |
| ATOM | 1091 | C | PHE | A | 139 | 2.484 | 71.099 | −34.217 | 1.00 | 19.57 | A |
| ATOM | 1092 | O | PHE | A | 139 | 3.373 | 70.842 | −35.028 | 1.00 | 20.33 | A |
| ATOM | 1093 | N | SER | A | 140 | 2.710 | 71.784 | −33.104 | 1.00 | 19.95 | A |
| ATOM | 1094 | CA | SER | A | 140 | 4.048 | 72.246 | −32.772 | 1.00 | 21.43 | A |
| ATOM | 1095 | CB | SER | A | 140 | 4.110 | 73.773 | −32.797 | 1.00 | 19.27 | A |
| ATOM | 1096 | OG | SER | A | 140 | 4.001 | 74.263 | −34.120 | 1.00 | 29.67 | A |
| ATOM | 1097 | C | SER | A | 140 | 4.483 | 71.757 | −31.403 | 1.00 | 22.54 | A |
| ATOM | 1098 | O | SER | A | 140 | 3.713 | 71.785 | −30.444 | 1.00 | 23.21 | A |
| ATOM | 1099 | N | ILE | A | 141 | 5.719 | 71.287 | −31.323 | 1.00 | 20.96 | A |
| ATOM | 1100 | CA | ILE | A | 141 | 6.275 | 70.836 | −30.061 | 1.00 | 21.83 | A |
| ATOM | 1101 | CB | ILE | A | 141 | 6.841 | 69.403 | −30.163 | 1.00 | 21.57 | A |
| ATOM | 1102 | CG2 | ILE | A | 141 | 7.575 | 69.041 | −28.877 | 1.00 | 19.32 | A |
| ATOM | 1103 | CG1 | ILE | A | 141 | 5.701 | 68.414 | −30.443 | 1.00 | 22.66 | A |
| ATOM | 1104 | CD1 | ILE | A | 141 | 6.163 | 66.997 | −30.780 | 1.00 | 17.77 | A |
| ATOM | 1105 | C | ILE | A | 141 | 7.407 | 71.809 | −29.749 | 1.00 | 22.95 | A |
| ATOM | 1106 | O | ILE | A | 141 | 8.451 | 71.789 | −30.404 | 1.00 | 21.65 | A |
| ATOM | 1107 | N | PRO | A | 142 | 7.198 | 72.703 | −28.768 | 1.00 | 23.79 | A |
| ATOM | 1108 | CD | PRO | A | 142 | 5.984 | 72.905 | −27.961 | 1.00 | 21.59 | A |
| ATOM | 1109 | CA | PRO | A | 142 | 8.234 | 73.673 | −28.401 | 1.00 | 26.75 | A |
| ATOM | 1110 | CB | PRO | A | 142 | 7.521 | 74.594 | −27.402 | 1.00 | 25.49 | A |
| ATOM | 1111 | CG | PRO | A | 142 | 6.052 | 74.379 | −27.680 | 1.00 | 24.33 | A |
| ATOM | 1112 | C | PRO | A | 142 | 9.377 | 72.911 | −27.739 | 1.00 | 28.74 | A |
| ATOM | 1113 | O | PRO | A | 142 | 9.135 | 72.040 | −26.909 | 1.00 | 34.61 | A |
| ATOM | 1114 | N | GLN | A | 143 | 10.613 | 73.227 | −28.103 | 1.00 | 30.75 | A |
| ATOM | 1115 | CA | GLN | A | 143 | 11.762 | 72.545 | −27.507 | 1.00 | 32.19 | A |
| ATOM | 1116 | CB | GLN | A | 143 | 12.096 | 73.168 | −26.147 | 1.00 | 32.70 | A |
| ATOM | 1117 | CG | GLN | A | 143 | 12.511 | 74.639 | −26.193 | 1.00 | 31.45 | A |
| ATOM | 1118 | CD | GLN | A | 143 | 11.327 | 75.584 | −26.276 | 1.00 | 33.00 | A |
| ATOM | 1119 | OE1 | GLN | A | 143 | 10.348 | 75.428 | −25.548 | 1.00 | 35.24 | A |
| ATOM | 1120 | NE2 | GLN | A | 143 | 11.418 | 76.581 | −27.154 | 1.00 | 32.90 | A |
| ATOM | 1121 | C | GLN | A | 143 | 11.548 | 71.034 | −27.328 | 1.00 | 31.26 | A |
| ATOM | 1122 | O | GLN | A | 143 | 11.171 | 70.568 | −26.250 | 1.00 | 31.29 | A |
| ATOM | 1123 | N | ALA | A | 144 | 11.807 | 70.271 | −28.382 | 1.00 | 27.26 | A |
| ATOM | 1124 | CA | ALA | A | 144 | 11.638 | 68.825 | −28.334 | 1.00 | 26.72 | A |
| ATOM | 1125 | CB | ALA | A | 144 | 11.566 | 68.269 | −29.751 | 1.00 | 23.73 | A |
| ATOM | 1126 | C | ALA | A | 144 | 12.755 | 68.127 | −27.562 | 1.00 | 27.14 | A |
| ATOM | 1127 | O | ALA | A | 144 | 13.877 | 68.622 | −27.477 | 1.00 | 28.39 | A |
| ATOM | 1128 | N | ASN | A | 145 | 12.444 | 66.973 | −26.987 | 1.00 | 27.44 | A |
| ATOM | 1129 | CA | ASN | A | 145 | 13.449 | 66.218 | −26.258 | 1.00 | 27.66 | A |
| ATOM | 1130 | CB | ASN | A | 145 | 13.356 | 66.493 | −24.751 | 1.00 | 27.80 | A |
| ATOM | 1131 | CG | ASN | A | 145 | 12.014 | 66.128 | −24.167 | 1.00 | 31.23 | A |
| ATOM | 1132 | OD1 | ASN | A | 145 | 11.572 | 64.984 | −24.271 | 1.00 | 31.16 | A |
| ATOM | 1133 | ND2 | ASN | A | 145 | 11.360 | 67.098 | −23.529 | 1.00 | 27.07 | A |
| ATOM | 1134 | C | ASN | A | 145 | 13.301 | 64.732 | −26.564 | 1.00 | 29.05 | A |
| ATOM | 1135 | O | ASN | A | 145 | 12.314 | 64.312 | −27.171 | 1.00 | 26.80 | A |
| ATOM | 1136 | N | HIS | A | 146 | 14.295 | 63.947 | −26.162 | 1.00 | 30.69 | A |
| ATOM | 1137 | CA | HIS | A | 146 | 14.302 | 62.509 | −26.415 | 1.00 | 32.92 | A |
| ATOM | 1138 | CB | HIS | A | 146 | 15.247 | 61.810 | −25.435 | 1.00 | 38.82 | A |
| ATOM | 1139 | CG | HIS | A | 146 | 16.691 | 61.932 | −25.807 | 1.00 | 44.79 | A |
| ATOM | 1140 | CD2 | HIS | A | 146 | 17.688 | 61.015 | −25.825 | 1.00 | 48.64 | A |
| ATOM | 1141 | ND1 | HIS | A | 146 | 17.254 | 63.119 | −26.224 | 1.00 | 47.95 | A |
| ATOM | 1142 | CE1 | HIS | A | 146 | 18.536 | 62.928 | −26.485 | 1.00 | 49.41 | A |
| ATOM | 1143 | NE2 | HIS | A | 146 | 18.824 | 61.660 | −26.250 | 1.00 | 50.75 | A |
| ATOM | 1144 | C | HIS | A | 146 | 12.942 | 61.833 | −26.375 | 1.00 | 30.36 | A |
| ATOM | 1145 | O | HIS | A | 146 | 12.645 | 60.991 | −27.217 | 1.00 | 29.00 | A |
| ATOM | 1146 | N | SER | A | 147 | 12.112 | 62.208 | −25.409 | 1.00 | 28.17 | A |
| ATOM | 1147 | CA | SER | A | 147 | 10.795 | 61.602 | −25.271 | 1.00 | 27.11 | A |
| ATOM | 1148 | CB | SER | A | 147 | 10.143 | 62.061 | −23.967 | 1.00 | 24.99 | A |
| ATOM | 1149 | OG | SER | A | 147 | 9.810 | 63.434 | −24.021 | 1.00 | 31.10 | A |
| ATOM | 1150 | C | SER | A | 147 | 9.841 | 61.868 | −26.441 | 1.00 | 24.50 | A |
| ATOM | 1151 | O | SER | A | 147 | 8.809 | 61.208 | −26.553 | 1.00 | 23.25 | A |
| ATOM | 1152 | N | HIS | A | 148 | 10.176 | 62.824 | −27.306 | 1.00 | 22.22 | A |
| ATOM | 1153 | CA | HIS | A | 148 | 9.321 | 63.142 | −28.452 | 1.00 | 21.23 | A |
| ATOM | 1154 | CB | HIS | A | 148 | 9.362 | 64.645 | −28.774 | 1.00 | 20.85 | A |
| ATOM | 1155 | CG | HIS | A | 148 | 8.738 | 65.503 | −27.717 | 1.00 | 24.24 | A |
| ATOM | 1156 | CD2 | HIS | A | 148 | 7.443 | 65.825 | −27.483 | 1.00 | 22.14 | A |
| ATOM | 1157 | ND1 | HIS | A | 148 | 9.469 | 66.095 | −26.709 | 1.00 | 22.36 | A |
| ATOM | 1158 | CE1 | HIS | A | 148 | 8.650 | 66.741 | −25.898 | 1.00 | 23.31 | A |
| ATOM | 1159 | NE2 | HIS | A | 148 | 7.415 | 66.592 | −26.345 | 1.00 | 22.83 | A |
| ATOM | 1160 | C | HIS | A | 148 | 9.684 | 62.340 | −29.701 | 1.00 | 20.35 | A |
| ATOM | 1161 | O | HIS | A | 148 | 8.982 | 62.415 | −30.709 | 1.00 | 17.82 | A |
| ATOM | 1162 | N | SER | A | 149 | 10.779 | 61.581 | −29.637 | 1.00 | 18.54 | A |
| ATOM | 1163 | CA | SER | A | 149 | 11.198 | 60.746 | −30.767 | 1.00 | 18.81 | A |
| ATOM | 1164 | CB | SER | A | 149 | 12.552 | 60.073 | −30.488 | 1.00 | 18.67 | A |
| ATOM | 1165 | OG | SER | A | 149 | 13.636 | 60.979 | −30.620 | 1.00 | 13.31 | A |
| ATOM | 1166 | C | SER | A | 149 | 10.154 | 59.659 | −30.971 | 1.00 | 21.07 | A |
| ATOM | 1167 | O | SER | A | 149 | 9.586 | 59.149 | −29.998 | 1.00 | 24.17 | A |
| ATOM | 1168 | N | GLY | A | 150 | 9.895 | 59.302 | −32.226 | 1.00 | 16.57 | A |
| ATOM | 1169 | CA | GLY | A | 150 | 8.919 | 58.264 | −32.487 | 1.00 | 15.96 | A |
| ATOM | 1170 | C | GLY | A | 150 | 8.277 | 58.341 | −33.856 | 1.00 | 16.19 | A |
| ATOM | 1171 | O | GLY | A | 150 | 8.663 | 59.157 | −34.688 | 1.00 | 13.71 | A |
| ATOM | 1172 | N | ASP | A | 151 | 7.295 | 57.472 | −34.082 | 1.00 | 17.34 | A |
| ATOM | 1173 | CA | ASP | A | 151 | 6.564 | 57.421 | −35.345 | 1.00 | 17.06 | A |
| ATOM | 1174 | CB | ASP | A | 151 | 6.063 | 56.001 | −35.615 | 1.00 | 18.28 | A |
| ATOM | 1175 | CG | ASP | A | 151 | 7.058 | 55.168 | −36.405 | 1.00 | 19.21 | A |
| ATOM | 1176 | OD1 | ASP | A | 151 | 8.272 | 55.467 | −36.366 | 1.00 | 17.94 | A |
| ATOM | 1177 | OD2 | ASP | A | 151 | 6.621 | 54.200 | −37.057 | 1.00 | 21.37 | A |
| ATOM | 1178 | C | ASP | A | 151 | 5.384 | 58.359 | −35.260 | 1.00 | 17.25 | A |
| ATOM | 1179 | O | ASP | A | 151 | 4.550 | 58.236 | −34.365 | 1.00 | 20.13 | A |
| ATOM | 1180 | N | TYR | A | 152 | 5.314 | 59.298 | −36.196 | 1.00 | 14.73 | A |
| ATOM | 1181 | CA | TYR | A | 152 | 4.232 | 60.262 | −36.212 | 1.00 | 14.30 | A |
| ATOM | 1182 | CB | TYR | A | 152 | 4.804 | 61.683 | −36.231 | 1.00 | 11.54 | A |
| ATOM | 1183 | CG | TYR | A | 152 | 5.329 | 62.160 | −34.898 | 1.00 | 12.47 | A |
| ATOM | 1184 | CD1 | TYR | A | 152 | 6.524 | 61.667 | −34.375 | 1.00 | 12.31 | A |
| ATOM | 1185 | CE1 | TYR | A | 152 | 6.989 | 62.084 | −33.130 | 1.00 | 12.64 | A |
| ATOM | 1186 | CD2 | TYR | A | 152 | 4.610 | 63.091 | −34.142 | 1.00 | 14.13 | A |
| ATOM | 1187 | CE2 | TYR | A | 152 | 5.062 | 63.516 | −32.896 | 1.00 | 14.51 | A |
| ATOM | 1188 | CZ | TYR | A | 152 | 6.249 | 63.008 | −32.394 | 1.00 | 15.75 | A |
| ATOM | 1189 | OH | TYR | A | 152 | 6.678 | 63.406 | −31.153 | 1.00 | 11.77 | A |
| ATOM | 1190 | C | TYR | A | 152 | 3.295 | 60.081 | −37.402 | 1.00 | 15.27 | A |
| ATOM | 1191 | O | TYR | A | 152 | 3.733 | 59.823 | −38.525 | 1.00 | 16.13 | A |
| ATOM | 1192 | N | HIS | A | 153 | 1.998 | 60.208 | −37.153 | 1.00 | 14.34 | A |
| ATOM | 1193 | CA | HIS | A | 153 | 1.018 | 60.109 | −38.228 | 1.00 | 15.41 | A |
| ATOM | 1194 | CB | HIS | A | 153 | 0.698 | 58.640 | −38.552 | 1.00 | 15.84 | A |
| ATOM | 1195 | CG | HIS | A | 153 | −0.326 | 58.019 | −37.653 | 1.00 | 13.70 | A |
| ATOM | 1196 | CD2 | HIS | A | 153 | −1.633 | 57.732 | −37.861 | 1.00 | 14.39 | A |
| ATOM | 1197 | ND1 | HIS | A | 153 | −0.038 | 57.591 | −36.375 | 1.00 | 15.08 | A |
| ATOM | 1198 | CE1 | HIS | A | 153 | −1.123 | 57.063 | −35.836 | 1.00 | 12.68 | A |
| ATOM | 1199 | NE2 | HIS | A | 153 | −2.103 | 57.135 | −36.718 | 1.00 | 17.11 | A |
| ATOM | 1200 | C | HIS | A | 153 | −0.237 | 60.857 | −37.794 | 1.00 | 14.86 | A |
| ATOM | 1201 | O | HIS | A | 153 | −0.328 | 61.291 | −36.652 | 1.00 | 16.11 | A |
| ATOM | 1202 | N | CYS | A | 154 | −1.199 | 61.023 | −38.693 | 1.00 | 16.61 | A |
| ATOM | 1203 | CA | CYS | A | 154 | −2.420 | 61.729 | −38.328 | 1.00 | 15.60 | A |
| ATOM | 1204 | C | CYS | A | 154 | −3.693 | 61.162 | −38.959 | 1.00 | 15.10 | A |
| ATOM | 1205 | O | CYS | A | 154 | −3.642 | 60.371 | −39.899 | 1.00 | 16.12 | A |
| ATOM | 1206 | CB | CYS | A | 154 | −2.289 | 63.221 | −38.678 | 1.00 | 15.38 | A |
| ATOM | 1207 | SG | CYS | A | 154 | −2.024 | 63.623 | −40.441 | 1.00 | 14.84 | A |
| ATOM | 1208 | N | THR | A | 155 | −4.831 | 61.553 | −38.393 | 1.00 | 10.96 | A |
| ATOM | 1209 | CA | THR | A | 155 | −6.137 | 61.162 | −38.895 | 1.00 | 10.42 | A |
| ATOM | 1210 | CB | THR | A | 155 | −6.891 | 60.183 | −37.939 | 1.00 | 12.01 | A |
| ATOM | 1211 | OG1 | THR | A | 155 | −7.015 | 60.767 | −36.638 | 1.00 | 14.89 | A |
| ATOM | 1212 | CG2 | THR | A | 155 | −6.155 | 58.858 | −37.824 | 1.00 | 11.03 | A |
| ATOM | 1213 | C | THR | A | 155 | −6.915 | 62.470 | −39.009 | 1.00 | 9.58 | A |
| ATOM | 1214 | O | THR | A | 155 | −6.601 | 63.449 | −38.334 | 1.00 | 9.61 | A |
| ATOM | 1215 | N | GLY | A | 156 | −7.912 | 62.495 | −39.875 | 1.00 | 11.28 | A |
| ATOM | 1216 | CA | GLY | A | 156 | −8.697 | 63.702 | −40.051 | 1.00 | 11.79 | A |
| ATOM | 1217 | C | GLY | A | 156 | −9.851 | 63.420 | −40.986 | 1.00 | 12.33 | A |
| ATOM | 1218 | O | GLY | A | 156 | −9.802 | 62.474 | −41.777 | 1.00 | 12.12 | A |
| ATOM | 1219 | N | ASN | A | 157 | −10.890 | 64.238 | −40.901 | 1.00 | 13.76 | A |
| ATOM | 1220 | CA | ASN | A | 157 | −12.064 | 64.051 | −41.745 | 1.00 | 16.88 | A |
| ATOM | 1221 | CB | ASN | A | 157 | −13.347 | 64.239 | −40.929 | 1.00 | 14.96 | A |
| ATOM | 1222 | CG | ASN | A | 157 | −13.500 | 63.210 | −39.843 | 1.00 | 18.62 | A |
| ATOM | 1223 | OD1 | ASN | A | 157 | −13.766 | 62.040 | −40.117 | 1.00 | 22.72 | A |
| ATOM | 1224 | ND2 | ASN | A | 157 | −13.322 | 63.634 | −38.596 | 1.00 | 17.93 | A |
| ATOM | 1225 | C | ASN | A | 157 | −12.121 | 64.991 | −42.934 | 1.00 | 15.84 | A |
| ATOM | 1226 | O | ASN | A | 157 | −12.003 | 66.205 | −42.792 | 1.00 | 16.29 | A |
| ATOM | 1227 | N | ILE | A | 158 | −12.283 | 64.415 | −44.112 | 1.00 | 16.86 | A |
| ATOM | 1228 | CA | ILE | A | 158 | −12.441 | 65.205 | −45.321 | 1.00 | 19.58 | A |
| ATOM | 1229 | CB | ILE | A | 158 | −11.483 | 64.763 | −46.448 | 1.00 | 23.46 | A |
| ATOM | 1230 | CG2 | ILE | A | 158 | −11.768 | 65.563 | −47.719 | 1.00 | 20.10 | A |
| ATOM | 1231 | CG1 | ILE | A | 158 | −10.034 | 65.009 | −46.013 | 1.00 | 20.85 | A |
| ATOM | 1232 | CD1 | ILE | A | 158 | −8.996 | 64.744 | −47.106 | 1.00 | 24.93 | A |
| ATOM | 1233 | C | ILE | A | 158 | −13.877 | 64.832 | −45.631 | 1.00 | 20.03 | A |
| ATOM | 1234 | O | ILE | A | 158 | −14.175 | 63.663 | −45.916 | 1.00 | 13.19 | A |
| ATOM | 1235 | N | GLY | A | 159 | −14.770 | 65.816 | −45.535 | 1.00 | 17.69 | A |
| ATOM | 1236 | CA | GLY | A | 159 | −16.173 | 65.533 | −45.730 | 1.00 | 19.70 | A |
| ATOM | 1237 | C | GLY | A | 159 | −16.531 | 64.727 | −44.493 | 1.00 | 21.42 | A |
| ATOM | 1238 | O | GLY | A | 159 | −16.289 | 65.174 | −43.368 | 1.00 | 22.94 | A |
| ATOM | 1239 | N | TYR | A | 160 | −17.069 | 63.530 | −44.687 | 1.00 | 20.65 | A |
| ATOM | 1240 | CA | TYR | A | 160 | −17.429 | 62.664 | −43.568 | 1.00 | 19.88 | A |
| ATOM | 1241 | CB | TYR | A | 160 | −18.884 | 62.208 | −43.705 | 1.00 | 24.20 | A |
| ATOM | 1242 | CG | TYR | A | 160 | −19.870 | 63.281 | −43.335 | 1.00 | 28.69 | A |
| ATOM | 1243 | CD1 | TYR | A | 160 | −20.011 | 63.690 | −42.006 | 1.00 | 32.29 | A |
| ATOM | 1244 | CE1 | TYR | A | 160 | −20.884 | 64.710 | −41.657 | 1.00 | 32.31 | A |
| ATOM | 1245 | CD2 | TYR | A | 160 | −20.633 | 63.921 | −44.307 | 1.00 | 29.62 | A |
| ATOM | 1246 | CE2 | TYR | A | 160 | −21.512 | 64.943 | −43.967 | 1.00 | 32.20 | A |
| ATOM | 1247 | CZ | TYR | A | 160 | −21.632 | 65.331 | −42.640 | 1.00 | 32.72 | A |
| ATOM | 1248 | OH | TYR | A | 160 | −22.505 | 66.334 | −42.296 | 1.00 | 38.30 | A |
| ATOM | 1249 | C | TYR | A | 160 | −16.522 | 61.440 | −43.481 | 1.00 | 17.88 | A |
| ATOM | 1250 | O | TYR | A | 160 | −16.797 | 60.513 | −42.732 | 1.00 | 15.53 | A |
| ATOM | 1251 | N | THR | A | 161 | −15.437 | 61.437 | −44.245 | 1.00 | 15.77 | A |
| ATOM | 1252 | CA | THR | A | 161 | −14.532 | 60.298 | −44.236 | 1.00 | 15.93 | A |
| ATOM | 1253 | CB | THR | A | 161 | −14.068 | 59.944 | −45.647 | 1.00 | 14.64 | A |
| ATOM | 1254 | OG1 | THR | A | 161 | −15.193 | 59.511 | −46.422 | 1.00 | 16.38 | A |
| ATOM | 1255 | CG2 | THR | A | 161 | −13.020 | 58.834 | −45.593 | 1.00 | 13.61 | A |
| ATOM | 1256 | C | THR | A | 161 | −13.304 | 60.543 | −43.387 | 1.00 | 15.68 | A |
| ATOM | 1257 | O | THR | A | 161 | −12.676 | 61.598 | −43.485 | 1.00 | 14.46 | A |
| ATOM | 1258 | N | LEU | A | 162 | −12.956 | 59.564 | −42.557 | 1.00 | 13.14 | A |
| ATOM | 1259 | CA | LEU | A | 162 | −11.790 | 59.708 | −41.708 | 1.00 | 18.10 | A |
| ATOM | 1260 | CB | LEU | A | 162 | −11.986 | 59.046 | −40.340 | 1.00 | 17.26 | A |
| ATOM | 1261 | CG | LEU | A | 162 | −10.716 | 59.285 | −39.502 | 1.00 | 18.64 | A |
| ATOM | 1262 | CD1 | LEU | A | 162 | −11.059 | 59.982 | −38.196 | 1.00 | 17.88 | A |
| ATOM | 1263 | CD2 | LEU | A | 162 | −9.997 | 57.975 | −39.266 | 1.00 | 13.35 | A |
| ATOM | 1264 | C | LEU | A | 162 | −10.567 | 59.109 | −42.363 | 1.00 | 19.66 | A |
| ATOM | 1265 | O | LEU | A | 162 | −10.394 | 57.888 | −42.397 | 1.00 | 19.82 | A |
| ATOM | 1266 | N | PHE | A | 163 | −9.713 | 59.978 | −42.883 | 1.00 | 17.71 | A |
| ATOM | 1267 | CA | PHE | A | 163 | −8.502 | 59.516 | −43.519 | 1.00 | 17.82 | A |
| ATOM | 1268 | CB | PHE | A | 163 | −8.136 | 60.423 | −44.695 | 1.00 | 19.31 | A |
| ATOM | 1269 | CG | PHE | A | 163 | −9.094 | 60.319 | −45.849 | 1.00 | 18.53 | A |
| ATOM | 1270 | CD1 | PHE | A | 163 | −9.976 | 61.356 | −46.139 | 1.00 | 18.60 | A |
| ATOM | 1271 | CD2 | PHE | A | 163 | −9.122 | 59.177 | −46.637 | 1.00 | 16.62 | A |
| ATOM | 1272 | CE1 | PHE | A | 163 | −10.874 | 61.258 | −47.203 | 1.00 | 14.79 | A |
| ATOM | 1273 | CE2 | PHE | A | 163 | −10.015 | 59.066 | −47.703 | 1.00 | 18.38 | A |
| ATOM | 1274 | CZ | PHE | A | 163 | −10.892 | 60.109 | −47.986 | 1.00 | 15.69 | A |
| ATOM | 1275 | C | PHE | A | 163 | −7.378 | 59.464 | −42.510 | 1.00 | 16.39 | A |
| ATOM | 1276 | O | PHE | A | 163 | −7.394 | 60.169 | −41.499 | 1.00 | 16.52 | A |
| ATOM | 1277 | N | SER | A | 164 | −6.407 | 58.612 | −42.795 | 1.00 | 16.90 | A |
| ATOM | 1278 | CA | SER | A | 164 | −5.262 | 58.424 | −41.929 | 1.00 | 17.36 | A |
| ATOM | 1279 | CB | SER | A | 164 | −5.382 | 57.083 | −41.213 | 1.00 | 17.26 | A |
| ATOM | 1280 | OG | SER | A | 164 | −4.173 | 56.780 | −40.544 | 1.00 | 23.66 | A |
| ATOM | 1281 | C | SER | A | 164 | −3.979 | 58.447 | −42.749 | 1.00 | 18.24 | A |
| ATOM | 1282 | O | SER | A | 164 | −3.908 | 57.824 | −43.813 | 1.00 | 14.99 | A |
| ATOM | 1283 | N | SER | A | 165 | −2.969 | 59.159 | −42.254 | 1.00 | 13.34 | A |
| ATOM | 1284 | CA | SER | A | 165 | −1.685 | 59.246 | −42.949 | 1.00 | 14.97 | A |
| ATOM | 1285 | CB | SER | A | 165 | −0.979 | 60.565 | −42.623 | 1.00 | 13.49 | A |
| ATOM | 1286 | OG | SER | A | 165 | −0.387 | 60.508 | −41.327 | 1.00 | 15.89 | A |
| ATOM | 1287 | C | SER | A | 165 | −0.770 | 58.111 | −42.512 | 1.00 | 13.99 | A |
| ATOM | 1288 | O | SER | A | 165 | −0.963 | 57.524 | −41.453 | 1.00 | 14.96 | A |
| ATOM | 1289 | N | LYS | A | 166 | 0.231 | 57.816 | −43.332 | 1.00 | 14.43 | A |
| ATOM | 1290 | CA | LYS | A | 166 | 1.207 | 56.783 | −43.011 | 1.00 | 17.24 | A |
| ATOM | 1291 | CB | LYS | A | 166 | 2.030 | 56.417 | −44.256 | 1.00 | 22.28 | A |
| ATOM | 1292 | CG | LYS | A | 166 | 1.263 | 55.718 | −45.370 | 1.00 | 24.97 | A |
| ATOM | 1293 | CD | LYS | A | 166 | 0.889 | 54.306 | −44.959 | 1.00 | 30.22 | A |
| ATOM | 1294 | CE | LYS | A | 166 | 0.198 | 53.546 | −46.087 | 1.00 | 30.56 | A |
| ATOM | 1295 | NZ | LYS | A | 166 | −0.025 | 52.118 | −45.713 | 1.00 | 33.39 | A |
| ATOM | 1296 | C | LYS | A | 166 | 2.140 | 57.386 | −41.961 | 1.00 | 19.02 | A |
| ATOM | 1297 | O | LYS | A | 166 | 2.257 | 58.615 | −41.849 | 1.00 | 17.29 | A |
| ATOM | 1298 | N | PRO | A | 167 | 2.823 | 56.540 | −41.179 | 1.00 | 19.04 | A |
| ATOM | 1299 | CD | PRO | A | 167 | 2.665 | 55.092 | −40.954 | 1.00 | 15.37 | A |
| ATOM | 1300 | CA | PRO | A | 167 | 3.717 | 57.137 | −40.181 | 1.00 | 18.46 | A |
| ATOM | 1301 | CB | PRO | A | 167 | 3.887 | 56.018 | −39.158 | 1.00 | 16.53 | A |
| ATOM | 1302 | CG | PRO | A | 167 | 3.808 | 54.780 | −40.004 | 1.00 | 17.02 | A |
| ATOM | 1303 | C | PRO | A | 167 | 5.054 | 57.599 | −40.765 | 1.00 | 17.73 | A |
| ATOM | 1304 | O | PRO | A | 167 | 5.492 | 57.121 | −41.812 | 1.00 | 19.57 | A |
| ATOM | 1305 | N | VAL | A | 168 | 5.676 | 58.557 | −40.086 | 1.00 | 16.85 | A |
| ATOM | 1306 | CA | VAL | A | 168 | 6.976 | 59.083 | −40.472 | 1.00 | 14.85 | A |
| ATOM | 1307 | CB | VAL | A | 168 | 6.868 | 60.527 | −41.029 | 1.00 | 19.26 | A |
| ATOM | 1308 | CG1 | VAL | A | 168 | 6.326 | 61.466 | −39.966 | 1.00 | 21.16 | A |
| ATOM | 1309 | CG2 | VAL | A | 168 | 8.233 | 61.001 | −41.515 | 1.00 | 19.06 | A |
| ATOM | 1310 | C | VAL | A | 168 | 7.790 | 59.066 | −39.179 | 1.00 | 13.79 | A |
| ATOM | 1311 | O | VAL | A | 168 | 7.353 | 59.606 | −38.166 | 1.00 | 12.28 | A |
| ATOM | 1312 | N | THR | A | 169 | 8.952 | 58.415 | −39.207 | 1.00 | 14.27 | A |
| ATOM | 1313 | CA | THR | A | 169 | 9.805 | 58.297 | −38.023 | 1.00 | 11.59 | A |
| ATOM | 1314 | CB | THR | A | 169 | 10.739 | 57.074 | −38.136 | 1.00 | 10.66 | A |
| ATOM | 1315 | OG1 | THR | A | 169 | 9.948 | 55.895 | −38.291 | 1.00 | 12.58 | A |
| ATOM | 1316 | CG2 | THR | A | 169 | 11.610 | 56.933 | −36.886 | 1.00 | 6.32 | A |
| ATOM | 1317 | C | THR | A | 169 | 10.661 | 59.533 | −37.775 | 1.00 | 14.44 | A |
| ATOM | 1318 | O | THR | A | 169 | 11.512 | 59.884 | −38.596 | 1.00 | 14.99 | A |
| ATOM | 1319 | N | ILE | A | 170 | 10.438 | 60.181 | −36.634 | 1.00 | 12.44 | A |
| ATOM | 1320 | CA | ILE | A | 170 | 11.189 | 61.377 | −36.284 | 1.00 | 14.17 | A |
| ATOM | 1321 | CB | ILE | A | 170 | 10.240 | 62.558 | −36.038 | 1.00 | 14.70 | A |
| ATOM | 1322 | CG2 | ILE | A | 170 | 11.029 | 63.771 | −35.539 | 1.00 | 15.74 | A |
| ATOM | 1323 | CG1 | ILE | A | 170 | 9.498 | 62.881 | −37.337 | 1.00 | 12.49 | A |
| ATOM | 1324 | CD1 | ILE | A | 170 | 8.493 | 63.986 | −37.212 | 1.00 | 19.93 | A |
| ATOM | 1325 | C | ILE | A | 170 | 12.078 | 61.165 | −35.063 | 1.00 | 15.27 | A |
| ATOM | 1326 | O | ILE | A | 170 | 11.640 | 60.620 | −34.047 | 1.00 | 15.24 | A |
| ATOM | 1327 | N | THR | A | 171 | 13.325 | 61.618 | −35.166 | 1.00 | 15.73 | A |
| ATOM | 1328 | CA | THR | A | 171 | 14.293 | 61.453 | −34.086 | 1.00 | 19.22 | A |
| ATOM | 1329 | CB | THR | A | 171 | 15.492 | 60.612 | −34.557 | 1.00 | 18.03 | A |
| ATOM | 1330 | OG1 | THR | A | 171 | 15.016 | 59.409 | −35.170 | 1.00 | 18.04 | A |
| ATOM | 1331 | CG2 | THR | A | 171 | 16.383 | 60.251 | −33.375 | 1.00 | 18.61 | A |
| ATOM | 1332 | C | THR | A | 171 | 14.830 | 62.763 | −33.514 | 1.00 | 18.58 | A |
| ATOM | 1333 | O | THR | A | 171 | 15.160 | 63.691 | −34.250 | 1.00 | 19.34 | A |
| ATOM | 1334 | N | VAL | A | 172 | 14.920 | 62.820 | −32.191 | 1.00 | 21.66 | A |
| ATOM | 1335 | CA | VAL | A | 172 | 15.427 | 64.000 | −31.504 | 1.00 | 24.01 | A |
| ATOM | 1336 | CB | VAL | A | 172 | 14.481 | 64.444 | −30.376 | 1.00 | 21.70 | A |
| ATOM | 1337 | CG1 | VAL | A | 172 | 15.029 | 65.702 | −29.708 | 1.00 | 19.02 | A |
| ATOM | 1338 | CG2 | VAL | A | 172 | 13.096 | 64.695 | −30.927 | 1.00 | 16.98 | A |
| ATOM | 1339 | C | VAL | A | 172 | 16.796 | 63.711 | −30.891 | 1.00 | 29.84 | A |
| ATOM | 1340 | O | VAL | A | 172 | 16.978 | 62.705 | −30.207 | 1.00 | 29.51 | A |
| ATOM | 1341 | N | GLN | A | 173 | 17.744 | 64.609 | −31.145 | 1.00 | 36.94 | A |
| ATOM | 1342 | CA | GLN | A | 173 | 19.113 | 64.510 | −30.642 | 1.00 | 42.47 | A |
| ATOM | 1343 | CB | GLN | A | 173 | 19.193 | 65.089 | −29.224 | 1.00 | 45.93 | A |
| ATOM | 1344 | CG | GLN | A | 173 | 20.608 | 65.463 | −28.773 | 1.00 | 50.02 | A |
| ATOM | 1345 | CD | GLN | A | 173 | 21.206 | 66.599 | −29.593 | 1.00 | 51.49 | A |
| ATOM | 1346 | OE1 | GLN | A | 173 | 22.348 | 67.003 | −29.377 | 1.00 | 53.09 | A |
| ATOM | 1347 | NE2 | GLN | A | 173 | 20.431 | 67.120 | −30.537 | 1.00 | 52.86 | A |
| ATOM | 1348 | C | GLN | A | 173 | 19.640 | 63.079 | −30.643 | 1.00 | 45.21 | A |
| ATOM | 1349 | O | GLN | A | 173 | 20.204 | 62.651 | −29.613 | 1.00 | 47.38 | A |
| ATOM | 1350 | OXT | GLN | A | 173 | 19.493 | 62.406 | −31.683 | 1.00 | 49.05 | A |
| ATOM | 1351 | O | HOH | S | 1 | 5.071 | 60.081 | −44.940 | 1.00 | 13.98 | S |
| ATOM | 1352 | O | HOH | S | 2 | 13.099 | 70.986 | −45.064 | 1.00 | 14.73 | S |
| ATOM | 1353 | O | HOH | S | 3 | 2.627 | 78.407 | −54.621 | 1.00 | 21.93 | S |
| ATOM | 1354 | O | HOH | S | 4 | −17.271 | 62.035 | −47.059 | 1.00 | 22.31 | S |
| ATOM | 1355 | O | HOH | S | 5 | −9.066 | 62.604 | −36.011 | 1.00 | 6.84 | S |
| ATOM | 1356 | O | HOH | S | 6 | −1.786 | 68.132 | −53.601 | 1.00 | 12.56 | S |
| ATOM | 1357 | O | HOH | S | 7 | 5.334 | 71.134 | −50.869 | 1.00 | 7.09 | S |
| ATOM | 1358 | O | HOH | S | 8 | −3.908 | 71.560 | −46.227 | 1.00 | 20.45 | S |
| ATOM | 1359 | O | HOH | S | 9 | 2.701 | 60.434 | −43.770 | 1.00 | 12.60 | S |
| ATOM | 1360 | O | HOH | S | 10 | 3.290 | 70.104 | −49.590 | 1.00 | 14.47 | S |
| ATOM | 1361 | O | HOH | S | 11 | −11.760 | 55.198 | −42.020 | 1.00 | 15.82 | S |
| ATOM | 1362 | O | HOH | S | 12 | 16.313 | 82.338 | −45.468 | 1.00 | 33.56 | S |
| ATOM | 1363 | O | HOH | S | 13 | 4.483 | 52.960 | −36.678 | 1.00 | 21.03 | S |
| ATOM | 1364 | O | HOH | S | 14 | −4.092 | 58.947 | −46.693 | 1.00 | 17.19 | S |
| ATOM | 1365 | O | HOH | S | 15 | 7.638 | 70.955 | −49.652 | 1.00 | 12.44 | S |
| ATOM | 1366 | O | HOH | S | 16 | 20.319 | 72.545 | −64.024 | 1.00 | 21.85 | S |
| ATOM | 1367 | O | HOH | S | 17 | 29.580 | 82.178 | −61.006 | 1.00 | 19.55 | S |
| ATOM | 1368 | O | HOH | S | 18 | 2.011 | 57.258 | −34.675 | 1.00 | 11.33 | S |
| ATOM | 1369 | O | HOH | S | 19 | 26.451 | 80.508 | −60.992 | 1.00 | 22.31 | S |
| ATOM | 1370 | O | HOH | S | 20 | 3.453 | 65.691 | −27.245 | 1.00 | 19.94 | S |
| ATOM | 1371 | O | HOH | S | 21 | 0.565 | 59.134 | −45.613 | 1.00 | 14.12 | S |
| ATOM | 1372 | O | HOH | S | 22 | 21.091 | 65.068 | −56.944 | 1.00 | 12.11 | S |
| ATOM | 1373 | O | HOH | S | 23 | 6.270 | 75.758 | −53.345 | 1.00 | 13.13 | S |
| ATOM | 1374 | O | HOH | S | 24 | −8.838 | 55.967 | −42.413 | 1.00 | 18.75 | S |
| ATOM | 1375 | O | HOH | S | 25 | 12.478 | 58.155 | −53.777 | 1.00 | 20.77 | S |
| ATOM | 1376 | O | HOH | S | 26 | 10.534 | 72.490 | −37.024 | 1.00 | 4.43 | S |
| ATOM | 1377 | O | HOH | S | 27 | 13.288 | 58.947 | −59.260 | 1.00 | 38.77 | S |
| ATOM | 1378 | O | HOH | S | 28 | 0.036 | 72.632 | −53.222 | 1.00 | 20.48 | S |
| ATOM | 1379 | O | HOH | S | 29 | 26.366 | 66.408 | −52.355 | 1.00 | 10.38 | S |
| ATOM | 1380 | O | HOH | S | 30 | 27.221 | 75.984 | −46.753 | 1.00 | 27.21 | S |
| ATOM | 1381 | O | HOH | S | 31 | 21.780 | 71.774 | −48.941 | 1.00 | 22.18 | S |
| ATOM | 1382 | O | HOH | S | 32 | 15.434 | 65.698 | −46.756 | 1.00 | 16.45 | S |
| ATOM | 1383 | O | HOH | S | 33 | 2.522 | 56.544 | −51.934 | 1.00 | 30.89 | S |
| ATOM | 1384 | O | HOH | S | 34 | −9.276 | 61.743 | −31.272 | 1.00 | 15.97 | S |
| ATOM | 1385 | O | HOH | S | 35 | 33.229 | 71.869 | −57.563 | 1.00 | 25.05 | S |
| ATOM | 1386 | O | HOH | S | 36 | 0.430 | 54.477 | −37.660 | 1.00 | 17.17 | S |
| ATOM | 1387 | O | HOH | S | 37 | 23.795 | 68.320 | −27.666 | 1.00 | 31.52 | S |
| ATOM | 1388 | O | HOH | S | 38 | 22.796 | 75.291 | −65.548 | 1.00 | 35.66 | S |
| ATOM | 1389 | O | HOH | S | 39 | 5.176 | 64.237 | −29.334 | 1.00 | 23.10 | S |
| ATOM | 1390 | O | HOH | S | 40 | 0.375 | 51.366 | −43.244 | 1.00 | 44.20 | S |
| ATOM | 1391 | O | HOH | S | 41 | 12.190 | 62.417 | −48.080 | 1.00 | 13.31 | S |
| ATOM | 1392 | O | HOH | S | 42 | 11.579 | 67.599 | −60.932 | 1.00 | 25.61 | S |
| ATOM | 1393 | O | HOH | S | 43 | 15.539 | 64.086 | −22.716 | 1.00 | 35.83 | S |
| ATOM | 1394 | O | HOH | S | 44 | −2.946 | 53.734 | −32.294 | 1.00 | 16.04 | S |
| ATOM | 1395 | O | HOH | S | 45 | 15.418 | 76.956 | −62.253 | 1.00 | 18.36 | S |
| ATOM | 1396 | O | HOH | S | 46 | 28.800 | 68.130 | −50.735 | 1.00 | 30.68 | S |
| ATOM | 1397 | O | HOH | S | 47 | 12.939 | 75.915 | −45.580 | 1.00 | 22.39 | S |
| ATOM | 1398 | O | HOH | S | 48 | −14.416 | 63.233 | −48.436 | 1.00 | 17.62 | S |
| ATOM | 1399 | O | HOH | S | 49 | −15.184 | 68.163 | −31.574 | 1.00 | 39.96 | S |
| ATOM | 1400 | O | HOH | S | 50 | 27.477 | 86.537 | −55.783 | 1.00 | 36.48 | S |
| ATOM | 1401 | O | HOH | S | 51 | −12.062 | 64.776 | −55.620 | 1.00 | 26.58 | S |
| ATOM | 1402 | O | HOH | S | 52 | 14.398 | 60.071 | −37.740 | 1.00 | 16.58 | S |
| ATOM | 1403 | O | HOH | S | 53 | 14.631 | 60.770 | −51.097 | 1.00 | 33.96 | S |
| ATOM | 1404 | O | HOH | S | 54 | −16.878 | 63.721 | −35.192 | 1.00 | 32.07 | S |
| ATOM | 1405 | O | HOH | S | 55 | 25.954 | 64.788 | −54.172 | 1.00 | 12.00 | S |
| ATOM | 1406 | O | HOH | S | 56 | 1.571 | 52.131 | −37.960 | 1.00 | 25.69 | S |
| ATOM | 1407 | O | HOH | S | 57 | 0.347 | 73.013 | −31.536 | 1.00 | 17.14 | S |
| ATOM | 1408 | O | HOH | S | 58 | −8.811 | 59.024 | −35.097 | 1.00 | 26.45 | S |
| ATOM | 1409 | O | HOH | S | 59 | 5.227 | 70.988 | −62.803 | 1.00 | 25.96 | S |
| ATOM | 1410 | O | HOH | S | 60 | 30.011 | 87.033 | −56.984 | 1.00 | 20.01 | S |
| ATOM | 1411 | O | HOH | S | 61 | 15.335 | 63.755 | −45.186 | 1.00 | 20.26 | S |
| ATOM | 1412 | O | HOH | S | 62 | 31.924 | 70.701 | −55.796 | 1.00 | 24.56 | S |
| ATOM | 1413 | O | HOH | S | 63 | 13.356 | 62.577 | −22.443 | 1.00 | 26.01 | S |
| ATOM | 1414 | O | HOH | S | 64 | 15.551 | 77.973 | −66.469 | 1.00 | 28.91 | S |
| ATOM | 1415 | O | HOH | S | 65 | 11.622 | 70.359 | −67.478 | 1.00 | 35.63 | S |
| ATOM | 1416 | O | HOH | S | 66 | 31.595 | 67.904 | −46.047 | 1.00 | 25.17 | S |
| ATOM | 1417 | O | HOH | S | 67 | 11.220 | 76.467 | −62.055 | 1.00 | 27.93 | S |
| ATOM | 1418 | O | HOH | S | 68 | 2.521 | 74.527 | −46.131 | 1.00 | 24.89 | S |
| ATOM | 1419 | O | HOH | S | 69 | −11.244 | 61.251 | −35.268 | 1.00 | 18.84 | S |
| ATOM | 1420 | O | HOH | S | 70 | 20.546 | 58.478 | −61.032 | 1.00 | 22.77 | S |
| ATOM | 1421 | O | HOH | S | 71 | −15.456 | 74.862 | −33.823 | 1.00 | 34.49 | S |
| ATOM | 1422 | O | HOH | S | 72 | −6.199 | 72.150 | −45.075 | 1.00 | 36.53 | S |
| ATOM | 1423 | O | HOH | S | 73 | 24.522 | 72.310 | −48.628 | 1.00 | 17.93 | S |
| ATOM | 1424 | O | HOH | S | 74 | −9.352 | 74.448 | −34.180 | 1.00 | 26.49 | S |
| ATOM | 1425 | O | HOH | S | 75 | 12.713 | 72.619 | −38.613 | 1.00 | 21.76 | S |
| ATOM | 1426 | O | HOH | S | 76 | 2.597 | 55.270 | −36.273 | 1.00 | 34.60 | S |
| ATOM | 1427 | O | HOH | S | 77 | −6.869 | 74.293 | −47.012 | 1.00 | 37.47 | S |
| ATOM | 1428 | O | HOH | S | 78 | −3.746 | 70.987 | −43.958 | 1.00 | 34.95 | S |
| ATOM | 1429 | O | HOH | S | 79 | 29.878 | 75.094 | −46.417 | 1.00 | 35.11 | S |
| ATOM | 1430 | O | HOH | S | 80 | 13.387 | 77.252 | −64.614 | 1.00 | 38.41 | S |
| ATOM | 1431 | O | HOH | S | 81 | −9.817 | 73.366 | −40.044 | 1.00 | 28.41 | S |
| ATOM | 1432 | O | HOH | S | 82 | 2.553 | 74.449 | −51.264 | 1.00 | 20.91 | S |
| ATOM | 1433 | O | HOH | S | 83 | 16.775 | 65.542 | −42.448 | 1.00 | 5.52 | S |
| ATOM | 1434 | O | HOH | S | 84 | −5.629 | 60.379 | −53.668 | 1.00 | 29.42 | S |
| ATOM | 1435 | O | HOH | S | 85 | 39.847 | 77.647 | −48.506 | 1.00 | 33.64 | S |
| END | | | | | | | | | | | |

### Example 4

### FcγRIIa Target Sites for Structure-Based Design of Therapeutic Compositions

Methods and Materials

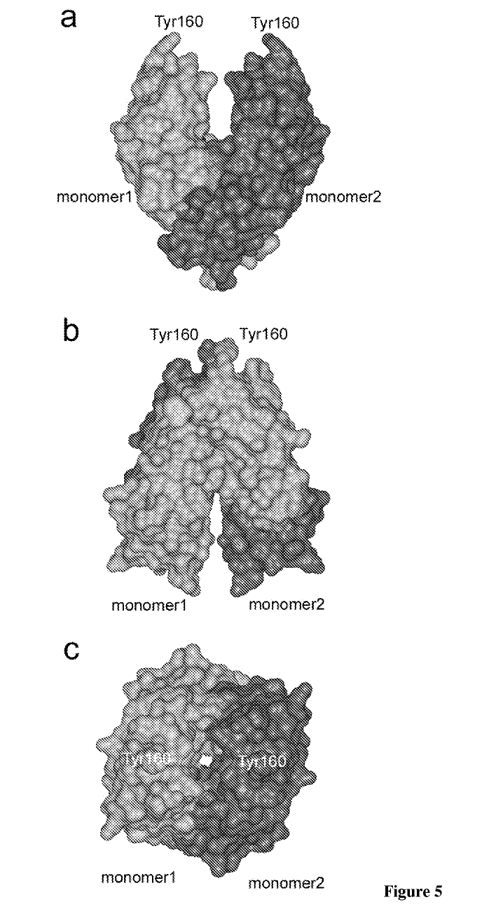

Various views of the HR_{S88} crystallographic dimer structure, as shown in FIGS. 5 to 7, were prepared using the Insight II program package, version 98.0 (Accelrys), and Connolly solvent-accessible surfaces are depicted (Connolly, 1983). Plots were generated with standard parameters using the LIGPLOT program (Wallace et al, 1995).

Results and Discussion

FIG. 5 illustrates the solvent-accessible surface views of the predominant crystallographic dimer of HR_{S88}, wherein the side-chain of Tyr160 is highlighted since it is a significant contributor to the receptor's binding site for antibodies and immune complexes. Examination of the three orthogonal views (FIG. 5) of the dimer of HR_{S88} revealed that a large solvent-filled groove exists between the receptor monomers. Further, a cavity and channel is formed in the lower portions of the juxtaposed surfaces of monomer 1 and monomer 2. The groove, cavity and channel represent novel target sites for agents for modulating the biological activity of FcR proteins, and particularly FcγRIIa. Such agents may be formulated into therapeutic compositions for, for example, inhibiting or stimulating FcγRIIa mediated inflammation.

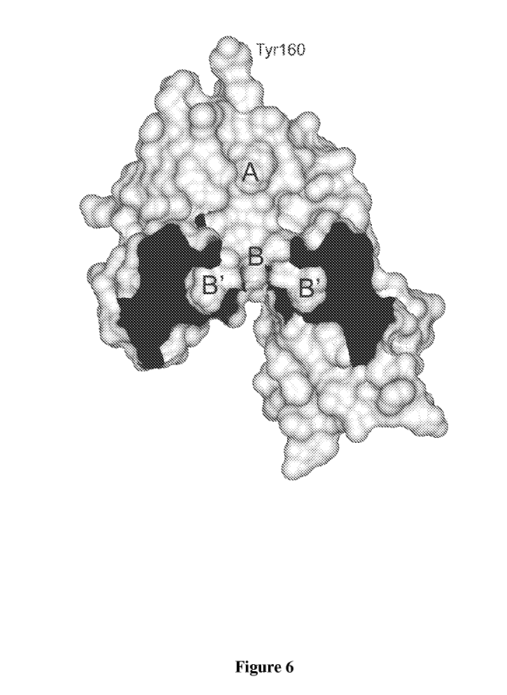

A cut-away diagram of the HR_{S88} dimer is shown in FIG. 6. Regions on each of the receptor monomers that are accessible (grey shaded surfaces) or inaccessible/buried (black shaded regions) to a solvent probe are shown. The buried regions are considered to form the interface between monomers 1 and 2. Juxtaposed surfaces of FcγRIIa monomers are considered to form suitable target sites for the structure-based design of agents using the provided atomic coordinates (Table 3). Target sites are identified in FIG. 6 as site A (a large groove between receptor monomers) and site B (a cavity and channel, located lower down near residues directly contributing to the monomer 1 monomer 2 interface). Site B consists of the central cavity/channel and two identical pockets designated as (B′). Agents which may comprise the active component of therapeutic compositions may specifically target site A or site B or, otherwise, bind simultaneously to sites A and B.

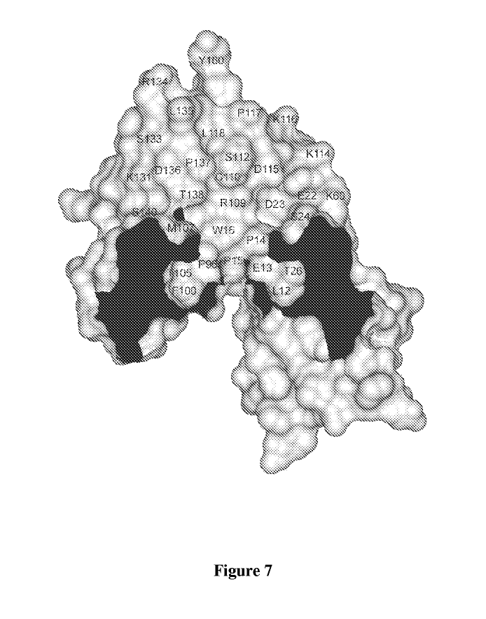

FIG. 7 illustrates the cut-away view of an HR_{S88} receptor monomer with the amino acid residues (in single letter code) contributing to the surfaces of the labelled target sites. Using the mapped surfaces (FIG. 7), the amino acid residues contributing to target sites A and B are defined as follows:
Site A is formed primarily by the following residues: Glu22, Asp23, Ser24, Lys60, Met107, Arg109, Cys110, Ser112, Lys114, Asp115, Lys116, Pro117, Leu118, Lys131, Ser133, Arg134, Leu135, Asp136, Pro137, Thr138, Ser140 and Tyr160, and
Site B is formed primarily by the following residues: (cavity and channel) Pro14, Pro15, Trp16; (B′ pockets) Leu12, Glu13, Thr26, Pro96, Phe100 and Thr105.

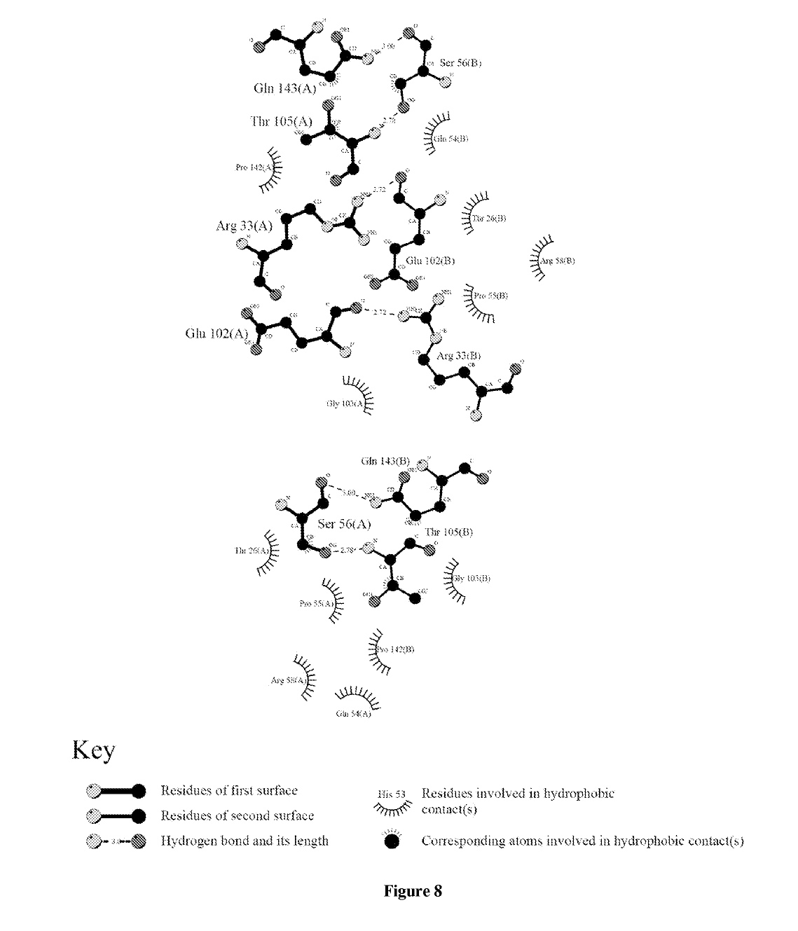

The amino acid residues that are directly involved in the formation of the interface between the receptor monomers (the “interface” residues) mostly form the black shaded regions on the cut-away solvent-accessible surface model (FIGS. 6 and 7). The interface residues on receptor monomer 1 (chain A) and monomer 2 (chain B) are shown in a schematic diagram (FIG. 8). Specifically, the amino acid residues directly contributing to the monomer 1: monomer 2 interface include:
Thr26, Arg33, Gln54, Pro55, Ser56, Arg58, Glu102, Gly103, Thr105, Pro142 and Gln143.

It is anticipated that altering interactions of the interface residues, either directly or indirectly, will contribute to the efficacy of therapeutic agent for inhibiting or stimulating FcγRIIa mediated inflammation. Direct effects are considered to occur when the agent interacts with at least one and usually more than one of the interface residues. Indirect effects are considered to occur through binding of an agent to sites adjacent or distant from the interface residues (eg target sites A and B, as defined above).

### Example 5

### Molecular Modelling of Active Anti-Inflammatory Compounds into the HR_{S88} Crystallographic Dimer

Methods and Materials

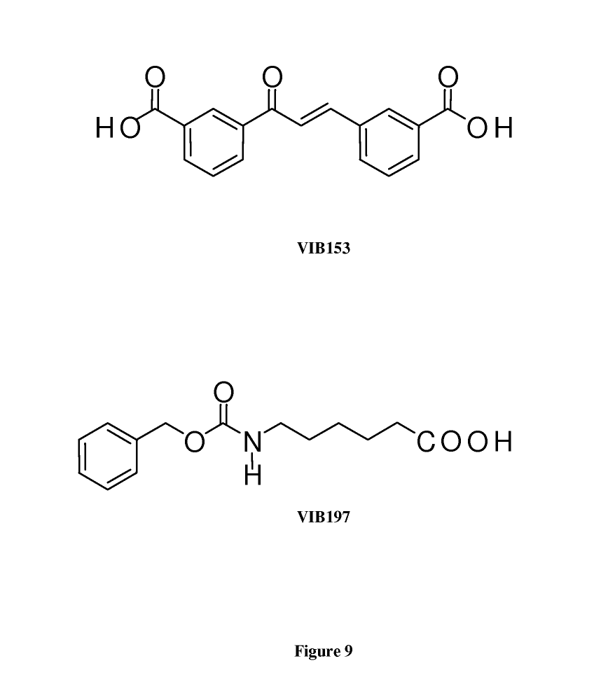

To examine whether the crystallographic dimer of HR_{S88} provided suitable surfaces for interacting with small chemical entities (SCE), molecular modelling was used to dock two compounds, designated as VIB153 and VIB197 (FIG. 9), into the defined target sites A and B. Both VIB153 and VIB197 have been previously shown to have inhibitory activity for FcγRIIa mediated inflammation (see International patent application no PCT/AU2003/001734 (Publication no WO 2004/058747), the entire disclosure of which is to be regarded as incorporated herein by reference).

For molecular modelling, ordered solvent atoms were first removed from the crystal coordinates of the dimer of HR_{S88}. Polar hydrogens were then added to HR_{S88} dimer structure. Ligand coordinate files (VIB153 and VIB197) were prepared in the standard Protein Data Bank (PDB) format (Berman et al, 2000). Ligand names were abbreviated to V53 (VIB153) and V97 (VIB197) since the PDB format only allows for three-letter residue names. Automated docking was performed using the Research algorithm, which is a Monte Carlo method using a pairwise van der Waals and electrostatic energy function (8 Å cutoff) and torsion sampling of the ligand conformational space (Hart et al, 1997). The energy function was used to rank all docked conformations of ligands after sampling 50 ligand conformers in 1000 trials. Target sites were defined by cubic grids (gridsteps of 0.5 Å) with 25 Å per side, centered on the following x, y, z realspace coordinates: Site A, x, y, z=0.68, 72.37, −45.70 (near Arg109) and; Site B, x, y, z=9.18, 74.17, −44.1 (near Pro15).

Results and Discussion

Atomic coordinates for the highest ranked (ie the lowest energy values) docked orientations of the VIB153 and VIB197 ligands into sites A and B of the HR_{S88} crystallographic dimer are provided in Tables 4 to 7).

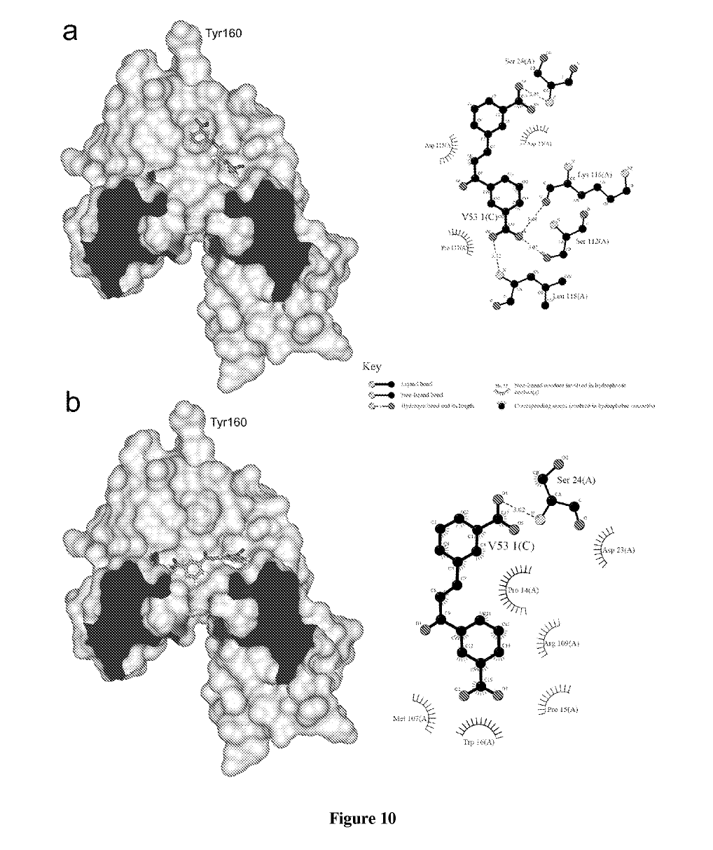

The predicted bound conformations of VIB153, at either target site, showed that the ligands predominantly interact with one of the monomers (chain A or monomer 1) of the HR_{S88} crystallographic dimer (FIG. 10). Binding of VIB153 to target site A, involves the two phenylcarboxylates entering into separate cavities on the surface lining the groove. Up to four hydrogen bonds are predicted between the protein constituents and the carboxylate moieties. The VIB153 ligand is further anchored by a series of hydrophobic van der Waals interactions (FIG. 10, panel a). Interactions between VIB153 and the target site B occur in the main cavity near the entry to the deep pockets and are predominantly hydrophobic in nature. Interestingly, one of the phenylcarboxylates binds into a pocket in the neighbouring groove (site A) and forms a hydrogen bond with Ser24 (FIG. 10, panel b). This same interaction was observed when VIB153 was docked directly into site A. Since SCEs like VIB153 often bind to proteins through a balance of hydrophobic and hydrogen bonding (electrostatic) interactions, the docking results indicate that target site A is preferred by this ligand.

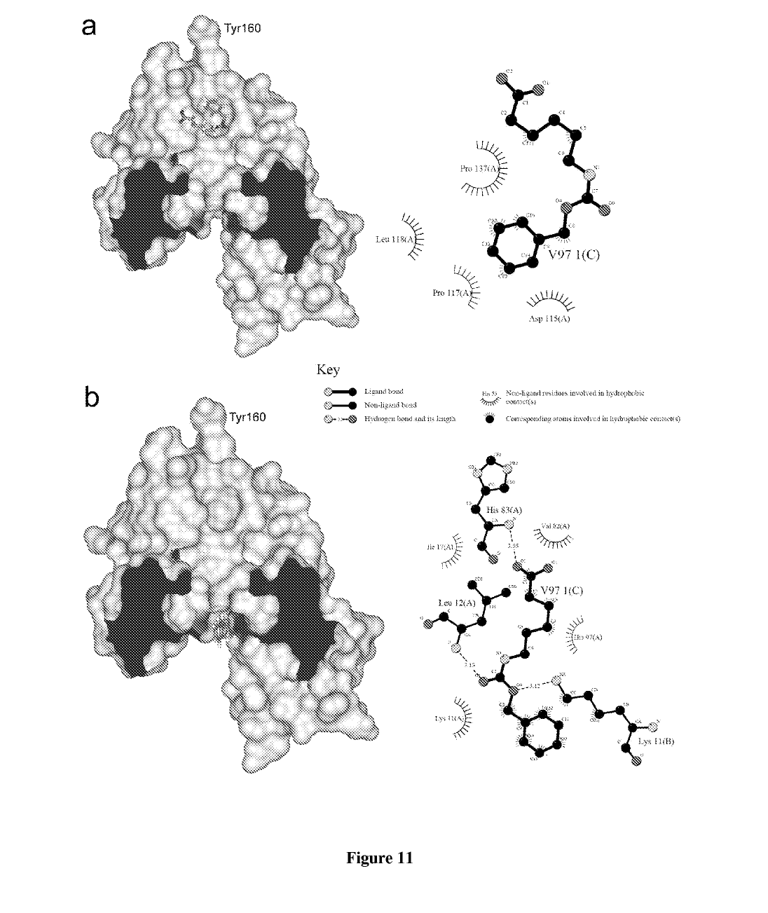

Automated docking of VIB197 into the target site A on the HR_{S88} crystallographic dimer also found that the ligand interacts exclusively with residues from monomer 1 (chain A). However, all interactions in the highest ranked bound conformation were hydrophobic in nature (FIG. 11, panel a). The potential hydrogen bonding donor and acceptor atoms of VIB197 were not utilised in binding to the groove. In contrast, when VIB197 was docked into site B, it bound with a mixed complement of hydrogen bonding and hydrophobic interactions with the protein (FIG. 11, panel b). While most interactions occur with monomer 1 (chain A), the side-chain of Lys1 of monomer 2 (chain B) is involved in a hydrogen bond and van der Waals interactions with the VIB197 ligand. Rather than binding at the cavity at the top of site B, VIB197 bound at the bottom of the channel of site B and in an adjacent groove at the edge of the dimer interface of monomer 1 (chain B). A comparison of the bound conformations in site A and site B indicates that VIB197 preferentially binds to target site B.

Collectively, the results of automated docking of VIB153 and VIB197 into the HR_{S88} crystallographic dimer, indicates that the possible mode of action of these compounds is to inhibit the formation of receptor dimers rather than directly inhibiting immune complex binding. In this regard, it was notable that all of the predicted bound conformations were located well away from the antibody binding site on FcγRIIa (surrounding the marked Tyr160 on FIGS. 10 and 11; also see FIGS. 4 and 5). Further, automated docking preferentially placed the ligands close to monomer 1 of the HR_{S88} crystallographic dimer. Thus, if the ligands bind to monomers of FcγRIIa, it is likely that they could have nearby or indirect effects, which alter or prevent interactions between the residues of the dimer interface. Interfering with the HR_{S88} dimer is proposed to reduce or eliminate signalling and concomitant inflammation that relies on the receptors clustering on the cell surface.

TABLE 4
Atomic coordinates for the highest ranked docked orientation of the
VIB153 ligand into site A of HR_{S88}-FcγRIIa crystallographic dimer

| | |
|---|---|
| REMARK ranking = | 1 |
| REMARK number of states = | 5 |
| REMARK Energy = | −34.4877 |

| | | | | | | | | | | |
|---|---|---|---|---|---|---|---|---|---|---|
| ATOM | 2701 | C1 | V53 | C | 1 | 1.007 | 73.788 | −50.639 | 1.00 | 20.00 |
| ATOM | 2702 | C2 | V53 | C | 1 | 1.801 | 74.827 | −51.130 | 1.00 | 20.00 |
| ATOM | 2703 | C3 | V53 | C | 1 | 1.635 | 76.123 | −50.641 | 1.00 | 20.00 |
| ATOM | 2704 | C4 | V53 | C | 1 | 0.674 | 76.384 | −49.661 | 1.00 | 20.00 |
| ATOM | 2705 | C5 | V53 | C | 1 | −0.132 | 75.359 | −49.153 | 1.00 | 20.00 |
| ATOM | 2706 | C6 | V53 | C | 1 | 0.045 | 74.061 | −49.657 | 1.00 | 20.00 |
| ATOM | 2707 | C7 | V53 | C | 1 | −1.152 | 75.729 | −48.088 | 1.00 | 20.00 |
| ATOM | 2708 | C8 | V53 | C | 1 | −2.488 | 75.719 | −48.219 | 1.00 | 20.00 |
| ATOM | 2709 | C9 | V53 | C | 1 | −3.407 | 76.097 | −47.118 | 1.00 | 20.00 |
| ATOM | 2710 | O1 | V53 | C | 1 | −3.811 | 77.248 | −47.097 | 1.00 | 20.00 |
| ATOM | 2711 | C10 | V53 | C | 1 | −3.885 | 75.163 | −46.017 | 1.00 | 20.00 |
| ATOM | 2712 | C11 | V53 | C | 1 | −3.319 | 75.294 | −44.743 | 1.00 | 20.00 |
| ATOM | 2713 | C12 | V53 | C | 1 | −4.866 | 74.176 | −46.201 | 1.00 | 20.00 |
| ATOM | 2714 | C13 | V53 | C | 1 | −5.271 | 73.341 | −45.151 | 1.00 | 20.00 |
| ATOM | 2715 | C14 | V53 | C | 1 | −4.690 | 73.493 | −43.891 | 1.00 | 20.00 |
| ATOM | 2716 | C15 | V53 | C | 1 | −3.715 | 74.469 | −43.687 | 1.00 | 20.00 |
| ATOM | 2717 | C16 | V53 | C | 1 | −6.331 | 72.282 | −45.376 | 1.00 | 20.00 |
| ATOM | 2718 | O2 | V53 | C | 1 | −7.238 | 72.140 | −44.508 | 1.00 | 20.00 |
| ATOM | 2719 | O3 | V53 | C | 1 | −6.273 | 71.574 | −46.422 | 1.00 | 20.00 |
| ATOM | 2720 | C17 | V53 | C | 1 | 1.186 | 72.379 | −51.168 | 1.00 | 20.00 |
| ATOM | 2721 | O4 | V53 | C | 1 | 1.721 | 71.511 | −50.421 | 1.00 | 20.00 |
| ATOM | 2722 | O5 | V53 | C | 1 | 0.792 | 72.119 | −52.342 | 1.00 | 20.00 |
| ATOM | 2723 | H1 | V53 | C | 1 | 2.551 | 74.639 | −51.892 | 1.00 | 0.00 |
| ATOM | 2724 | H2 | V53 | C | 1 | 2.252 | 76.929 | −51.023 | 1.00 | 0.00 |
| ATOM | 2725 | H3 | V53 | C | 1 | 0.565 | 77.401 | −49.299 | 1.00 | 0.00 |
| ATOM | 2726 | H4 | V53 | C | 1 | −0.550 | 73.238 | −49.297 | 1.00 | 0.00 |
| ATOM | 2727 | H5 | V53 | C | 1 | −0.747 | 76.032 | −47.122 | 1.00 | 0.00 |
| ATOM | 2728 | H6 | V53 | C | 1 | −2.917 | 75.421 | −49.165 | 1.00 | 0.00 |
| ATOM | 2729 | H7 | V53 | C | 1 | −2.558 | 76.044 | −44.552 | 1.00 | 0.00 |
| ATOM | 2730 | H8 | V53 | C | 1 | −5.333 | 74.035 | −47.163 | 1.00 | 0.00 |
| ATOM | 2731 | H9 | V53 | C | 1 | −4.989 | 72.858 | −43.062 | 1.00 | 0.00 |
| ATOM | 2732 | H10 | V53 | C | 1 | −3.263 | 74.587 | −42.706 | 1.00 | 0.00 |
| ATOM | 2733 | H11 | V53 | C | 1 | −6.979 | 70.870 | −46.568 | 1.00 | 0.00 |
| ATOM | 2734 | H12 | V53 | C | 1 | 0.916 | 71.180 | −52.687 | 1.00 | 0.00 |
| END | | | | | | | | | | |

TABLE 5
Atomic coordinates for the highest ranked docked orientation of the
VIB153 ligand into site B of HR_{S88}-FcγRIIa crystallographic dimer

| | |
|---|---|
| REMARK ranking = | 1 |
| REMARK number of states = | 1 |
| REMARK Energy = | −34.1727 |

| | | | | | | | | | | |
|---|---|---|---|---|---|---|---|---|---|---|
| ATOM | 2701 | C1 | V53 | C | 1 | 2.299 | 73.789 | −50.687 | 1.00 | 20.00 |
| ATOM | 2702 | C2 | V53 | C | 1 | 2.664 | 74.654 | −51.723 | 1.00 | 20.00 |
| ATOM | 2703 | C3 | V53 | C | 1 | 2.929 | 75.996 | −51.449 | 1.00 | 20.00 |
| ATOM | 2704 | C4 | V53 | C | 1 | 2.831 | 76.477 | −50.142 | 1.00 | 20.00 |
| ATOM | 2705 | C5 | V53 | C | 1 | 2.466 | 75.631 | −49.086 | 1.00 | 20.00 |
| ATOM | 2706 | C6 | V53 | C | 1 | 2.202 | 74.283 | −49.380 | 1.00 | 20.00 |
| ATOM | 2707 | C7 | V53 | C | 1 | 2.383 | 76.236 | −47.694 | 1.00 | 20.00 |
| ATOM | 2708 | C8 | V53 | C | 1 | 2.534 | 75.588 | −46.528 | 1.00 | 20.00 |
| ATOM | 2709 | C9 | V53 | C | 1 | 2.436 | 76.266 | −45.212 | 1.00 | 20.00 |
| ATOM | 2710 | O1 | V53 | C | 1 | 1.481 | 77.003 | −45.033 | 1.00 | 20.00 |
| ATOM | 2711 | C10 | V53 | C | 1 | 3.431 | 76.114 | −44.072 | 1.00 | 20.00 |
| ATOM | 2712 | C11 | V53 | C | 1 | 4.794 | 76.256 | −44.358 | 1.00 | 20.00 |
| ATOM | 2713 | C12 | V53 | C | 1 | 3.062 | 75.842 | −42.746 | 1.00 | 20.00 |
| ATOM | 2714 | C13 | V53 | C | 1 | 4.020 | 75.712 | −41.731 | 1.00 | 20.00 |
| ATOM | 2715 | C14 | V53 | C | 1 | 5.373 | 75.858 | −42.043 | 1.00 | 20.00 |
| ATOM | 2716 | C15 | V53 | C | 1 | 5.759 | 76.130 | −43.356 | 1.00 | 20.00 |
| ATOM | 2717 | C16 | V53 | C | 1 | 3.597 | 75.417 | −40.307 | 1.00 | 20.00 |
| ATOM | 2718 | O2 | V53 | C | 1 | 2.716 | 76.148 | −39.773 | 1.00 | 20.00 |
| ATOM | 2719 | O3 | V53 | C | 1 | 4.139 | 74.449 | −39.699 | 1.00 | 20.00 |
| ATOM | 2720 | C17 | V53 | C | 1 | 2.010 | 72.331 | −50.982 | 1.00 | 20.00 |
| ATOM | 2721 | O4 | V53 | C | 1 | 1.246 | 72.044 | −51.947 | 1.00 | 20.00 |
| ATOM | 2722 | O5 | V53 | C | 1 | 2.545 | 71.447 | −50.250 | 1.00 | 20.00 |
| ATOM | 2723 | H1 | V53 | C | 1 | 2.743 | 74.294 | −52.743 | 1.00 | 0.00 |
| ATOM | 2724 | H2 | V53 | C | 1 | 3.212 | 76.667 | −52.254 | 1.00 | 0.00 |
| ATOM | 2725 | H3 | V53 | C | 1 | 3.042 | 77.526 | −49.958 | 1.00 | 0.00 |
| ATOM | 2726 | H4 | V53 | C | 1 | 1.918 | 73.592 | −48.601 | 1.00 | 0.00 |
| ATOM | 2727 | H5 | V53 | C | 1 | 2.180 | 77.306 | −47.648 | 1.00 | 0.00 |
| ATOM | 2728 | H6 | V53 | C | 1 | 2.738 | 74.527 | −46.543 | 1.00 | 0.00 |
| ATOM | 2729 | H7 | V53 | C | 1 | 5.128 | 76.468 | −45.369 | 1.00 | 0.00 |
| ATOM | 2730 | H8 | V53 | C | 1 | 2.025 | 75.724 | −42.473 | 1.00 | 0.00 |
| ATOM | 2731 | H9 | V53 | C | 1 | 6.134 | 75.762 | −41.273 | 1.00 | 0.00 |
| ATOM | 2732 | H10 | V53 | C | 1 | 6.813 | 76.243 | −43.598 | 1.00 | 0.00 |
| ATOM | 2733 | H11 | V53 | C | 1 | 3.857 | 74.256 | −38.751 | 1.00 | 0.00 |
| ATOM | 2734 | H12 | V53 | C | 1 | 2.347 | 70.481 | −50.453 | 1.00 | 0.00 |
| END | | | | | | | | | | |

TABLE 6
Atomic coordinates for the highest ranked docked orientation of the
VIB197 ligand into site A of HR_{S88}-FcγRIIa crystallographic dimer

| | |
|---|---|
| REMARK ranking = | 1 |
| REMARK number of states = | 2 |
| REMARK Energy = | −4.9971 |

| | | | | | | | | | | |
|---|---|---|---|---|---|---|---|---|---|---|
| ATOM | 2701 | O1 | V97 | C | 1 | −4.498 | 77.334 | −37.516 | 1.00 | 20.00 |
| ATOM | 2702 | C1 | V97 | C | 1 | −5.264 | 76.805 | −38.286 | 1.00 | 20.00 |
| ATOM | 2703 | O2 | V97 | C | 1 | −6.291 | 76.079 | −37.818 | 1.00 | 20.00 |
| ATOM | 2704 | C2 | V97 | C | 1 | −5.070 | 76.965 | −39.772 | 1.00 | 20.00 |
| ATOM | 2705 | C3 | V97 | C | 1 | −3.862 | 76.141 | −40.220 | 1.00 | 20.00 |
| ATOM | 2706 | C4 | V97 | C | 1 | −3.675 | 76.290 | −41.731 | 1.00 | 20.00 |
| ATOM | 2707 | C5 | V97 | C | 1 | −2.819 | 75.135 | −42.256 | 1.00 | 20.00 |
| ATOM | 2708 | C6 | V97 | C | 1 | −2.920 | 75.076 | −43.782 | 1.00 | 20.00 |
| ATOM | 2709 | N1 | V97 | C | 1 | −3.311 | 73.726 | −44.193 | 1.00 | 20.00 |
| ATOM | 2710 | C7 | V97 | C | 1 | −3.562 | 73.465 | −45.491 | 1.00 | 20.00 |
| ATOM | 2711 | O3 | V97 | C | 1 | −3.463 | 74.348 | −46.320 | 1.00 | 20.00 |
| ATOM | 2712 | O4 | V97 | C | 1 | −3.922 | 72.224 | −45.870 | 1.00 | 20.00 |
| ATOM | 2713 | C8 | V97 | C | 1 | −5.273 | 71.970 | −46.336 | 1.00 | 20.00 |
| ATOM | 2714 | C9 | V97 | C | 1 | −6.249 | 72.233 | −45.217 | 1.00 | 20.00 |
| ATOM | 2715 | C10 | V97 | C | 1 | −6.117 | 71.565 | −44.014 | 1.00 | 20.00 |
| ATOM | 2716 | C11 | V97 | C | 1 | −7.013 | 71.806 | −42.989 | 1.00 | 20.00 |
| ATOM | 2717 | C12 | V97 | C | 1 | −8.039 | 72.715 | −43.166 | 1.00 | 20.00 |
| ATOM | 2718 | C13 | V97 | C | 1 | −8.170 | 73.383 | −44.369 | 1.00 | 20.00 |
| ATOM | 2719 | C14 | V97 | C | 1 | −7.272 | 73.145 | −45.393 | 1.00 | 20.00 |
| ATOM | 2720 | H1 | V97 | C | 1 | −6.916 | 75.670 | −38.432 | 0.00 | 0.00 |
| ATOM | 2721 | H2 | V97 | C | 1 | −5.962 | 76.617 | −40.294 | 0.00 | 0.00 |
| ATOM | 2722 | H3 | V97 | C | 1 | −4.900 | 78.016 | −40.005 | 0.00 | 0.00 |
| ATOM | 2723 | H4 | V97 | C | 1 | −2.969 | 76.497 | −39.708 | 0.00 | 0.00 |
| ATOM | 2724 | H5 | V97 | C | 1 | −4.027 | 75.091 | −39.976 | 0.00 | 0.00 |
| ATOM | 2725 | H6 | V97 | C | 1 | −4.648 | 76.271 | −42.221 | 0.00 | 0.00 |
| ATOM | 2726 | H7 | V97 | C | 1 | −3.179 | 77.236 | −41.945 | 0.00 | 0.00 |
| ATOM | 2727 | H8 | V97 | C | 1 | −1.779 | 75.293 | −41.967 | 0.00 | 0.00 |
| ATOM | 2728 | H9 | V97 | C | 1 | −3.175 | 74.197 | −41.831 | 0.00 | 0.00 |
| ATOM | 2729 | H10 | V97 | C | 1 | −3.667 | 75.792 | −44.124 | 0.00 | 0.00 |
| ATOM | 2730 | H11 | V97 | C | 1 | −1.952 | 75.324 | −44.220 | 0.00 | 0.00 |
| ATOM | 2731 | H12 | V97 | C | 1 | −3.390 | 73.022 | −43.532 | 0.00 | 0.00 |
| ATOM | 2732 | H13 | V97 | C | 1 | −5.499 | 72.628 | −47.176 | 0.00 | 0.00 |
| ATOM | 2733 | H14 | V97 | C | 1 | −5.358 | 70.931 | −46.656 | 0.00 | 0.00 |
| ATOM | 2734 | H15 | V97 | C | 1 | −5.316 | 70.855 | −43.876 | 0.00 | 0.00 |
| ATOM | 2735 | H16 | V97 | C | 1 | −6.910 | 71.283 | −42.049 | 0.00 | 0.00 |
| ATOM | 2736 | H17 | V97 | C | 1 | −8.739 | 72.902 | −42.365 | 0.00 | 0.00 |
| ATOM | 2737 | H18 | V97 | C | 1 | −8.971 | 74.093 | −44.508 | 0.00 | 0.00 |
| ATOM | 2738 | H19 | V97 | C | 1 | −7.374 | 73.668 | −46.334 | 0.00 | 0.00 |
| END | | | | | | | | | | |

TABLE 7
Atomic coordinates for the highest ranked docked orientation of the
VIB197 ligand into site B of HR_{S88}-FcγRIIa crystallographic dimer

| | |
|---|---|
| REMARK ranking = | 1 |
| REMARK number of states = | 1 |
| REMARK Energy = | −35.0158 |

| | | | | | | | | | | |
|---|---|---|---|---|---|---|---|---|---|---|
| ATOM | 2701 | O1 | V97 | C | 1 | 16.046 | 66.037 | −46.733 | 1.00 | 20.00 |
| ATOM | 2702 | C1 | V97 | C | 1 | 15.941 | 67.032 | −46.055 | 1.00 | 20.00 |
| ATOM | 2703 | O2 | V97 | C | 1 | 15.271 | 66.984 | −44.894 | 1.00 | 20.00 |
| ATOM | 2704 | C2 | V97 | C | 1 | 16.567 | 68.324 | −46.511 | 1.00 | 20.00 |
| ATOM | 2705 | C3 | V97 | C | 1 | 15.614 | 69.484 | −46.216 | 1.00 | 20.00 |
| ATOM | 2706 | C4 | V97 | C | 1 | 16.386 | 70.619 | −45.540 | 1.00 | 20.00 |
| ATOM | 2707 | C5 | V97 | C | 1 | 15.796 | 71.964 | −45.968 | 1.00 | 20.00 |
| ATOM | 2708 | C6 | V97 | C | 1 | 14.382 | 72.106 | −45.401 | 1.00 | 20.00 |
| ATOM | 2709 | N1 | V97 | C | 1 | 14.333 | 73.253 | −44.492 | 1.00 | 20.00 |
| ATOM | 2710 | C7 | V97 | C | 1 | 14.382 | 74.506 | −44.985 | 1.00 | 20.00 |
| ATOM | 2711 | O3 | V97 | C | 1 | 14.465 | 74.685 | −46.184 | 1.00 | 20.00 |
| ATOM | 2712 | O4 | V97 | C | 1 | 14.337 | 75.560 | −44.149 | 1.00 | 20.00 |
| ATOM | 2713 | C8 | V97 | C | 1 | 13.853 | 76.844 | −44.624 | 1.00 | 20.00 |
| ATOM | 2714 | C9 | V97 | C | 1 | 14.739 | 77.943 | −44.098 | 1.00 | 20.00 |
| ATOM | 2715 | C10 | V97 | C | 1 | 16.113 | 77.825 | −44.187 | 1.00 | 20.00 |
| ATOM | 2716 | C11 | V97 | C | 1 | 16.926 | 78.833 | −43.702 | 1.00 | 20.00 |
| ATOM | 2717 | C12 | V97 | C | 1 | 16.365 | 79.959 | −43.129 | 1.00 | 20.00 |
| ATOM | 2718 | C13 | V97 | C | 1 | 14.990 | 80.077 | −43.040 | 1.00 | 20.00 |
| ATOM | 2719 | C14 | V97 | C | 1 | 14.177 | 79.070 | −43.529 | 1.00 | 20.00 |
| ATOM | 2720 | H1 | V97 | C | 1 | 14.740 | 67.740 | −44.609 | 0.00 | 0.00 |
| ATOM | 2721 | H2 | V97 | C | 1 | 17.506 | 68.481 | −45.980 | 0.00 | 0.00 |
| ATOM | 2722 | H3 | V97 | C | 1 | 16.760 | 68.276 | −47.583 | 0.00 | 0.00 |
| ATOM | 2723 | H4 | V97 | C | 1 | 15.181 | 69.846 | −47.149 | 0.00 | 0.00 |
| ATOM | 2724 | H5 | V97 | C | 1 | 14.817 | 69.142 | −45.555 | 0.00 | 0.00 |
| ATOM | 2725 | H6 | V97 | C | 1 | 16.307 | 70.517 | −44.458 | 0.00 | 0.00 |
| ATOM | 2726 | H7 | V97 | C | 1 | 17.434 | 70.571 | −45.834 | 0.00 | 0.00 |
| ATOM | 2727 | H8 | V97 | C | 1 | 16.422 | 72.773 | −45.589 | 0.00 | 0.00 |
| ATOM | 2728 | H9 | V97 | C | 1 | 15.757 | 72.014 | −47.056 | 0.00 | 0.00 |
| ATOM | 2729 | H10 | V97 | C | 1 | 13.676 | 72.258 | −46.218 | 0.00 | 0.00 |
| ATOM | 2730 | H11 | V97 | C | 1 | 14.115 | 71.198 | −44.857 | 0.00 | 0.00 |
| ATOM | 2731 | H12 | V97 | C | 1 | 14.266 | 73.110 | −43.535 | 0.00 | 0.00 |
| ATOM | 2732 | H13 | V97 | C | 1 | 13.868 | 76.857 | −45.715 | 0.00 | 0.00 |
| ATOM | 2733 | H14 | V97 | C | 1 | 12.832 | 77.000 | −44.274 | 0.00 | 0.00 |
| ATOM | 2734 | H15 | V97 | C | 1 | 16.552 | 76.946 | −44.635 | 0.00 | 0.00 |
| ATOM | 2735 | H16 | V97 | C | 1 | 18.000 | 78.741 | −43.772 | 0.00 | 0.00 |
| ATOM | 2736 | H17 | V97 | C | 1 | 16.999 | 80.746 | −42.750 | 0.00 | 0.00 |
| ATOM | 2737 | H18 | V97 | C | 1 | 14.551 | 80.955 | −42.593 | 0.00 | 0.00 |
| ATOM | 2738 | H19 | V97 | C | 1 | 13.103 | 79.163 | −43.459 | 0.00 | 0.00 |
| END | | | | | | | | | | |

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

Bartlett, P A et al, “CAVEAT: A Program to Facilitate the Structure-Derived Design of Biologically Active Molecules.” In Molecular Recognition in Chemical and Biological Problems, Special Pub., Royal Chem Soc, 78, pp 182-196, 1989.
Berman, H M et al, “The Protein Data Bank.” Nucleic Acids Res 28(1): 235-242, 2000.
Bohm, H-J, “The computer program LUD1: a new method for the de novo design of enzyme inhibitions”. J. Comp. Aid. Molec. Design, 6: 61-78, 1992.
Brunger, A T, et al, “Crystallography & NMR system: A new software suite for macromolecular structure determination.” Acta Crystallogr D54(Pt 5): 905-921, 1998.
Cohen, N C et al, “Molecular modelling software and methods for medicinal chemistry”. J. Med. Chem, 33: 883-894, 1990.
Connolly, M L “Analytical molecular surface calculation.” Journal of Applied Crystallography, 16: 548-558, 1983.
Goodford, P J, “A computational procedure for determining energetically favourable binding sites on biologically important macromolecules”. J. Med. Chem., 28: 849-857, 1985.
Goodsell D S et al, “Automated docking of substrates to proteins by simulated annealing”. Proteins: Structure, Function, and Genetics, 8: 195-202, 1990.
Hart, T N et al, “Critical evaluation of the research docking program for the CASP2 challenge.” Proteins, Suppl 1: 205-209, 1997.
Hogarth, P M, “Fc receptors are major mediators of antibody based inflammation in autoimmunity.” Curr Opin Immunol, 14(6): 798-802, 2002.
Kuntz I D et al, “A geometric approach to macromolecule-ligand interactions”. J. Mol. Biol, 161: 269-288, 1982.
Martin, Y C, “3D database searching in drug design”. J. Med. Chem, 35: 2145-2154, 1992.
Maxwell, K F et al, “Crystal structure of the human leukocyte Fc receptor, Fc gammaRIIa.” Nat Struct Biol, 6(5): 437-442, 1999.
Meng, E C et al, “Automated docking with grid-based energy evaluation”. J. Comp. Chem, 13: 505-524, 1992.
Nishibata, Y et al, “Automatic creation of drug candidate structures based on receptor structure: starting point for artificial lead generation”. Tetrahedron, 47: 8985, 1991.
Miranker, A et al, “Functionality maps of binding sites: a multiple copy simultaneous search method”. Proteins: Structure, Function and Genetics, 11: 29-34, 1991.
Navia, M A et al, “Use of structural information in drug design”. Current Opinion in Structural Biology, 2: 202-210, 1992
Parren, P W et al, “On the interaction of IgG subclasses with the low affinity Fc gamma RIIa (CD32) on human monocytes, neutrophils, and platelets. Analysis of a functional polymorphism to human IgG2.” J Clin Invest, 90(4): 1537-1546, 1992.
Powell, M S et al, “Biochemical analysis and crystallisation of Fc gamma RIIa, the low affinity receptor for IgG.” Immunol Lett, 68(1): 17-23, 1999.
Sautes, C N et al, “Soluble Fc gamma receptors II (Fc gamma RII) are generated by cleavage of membrane Fc gamma RII.” Eur J Immunol., 21(1): 231-234, 1991.
Sondermann, P et al, “The 3.2-A crystal structure of the human IgG1 Fc fragment-Fc gammaRIII complex.” Nature, 406(6793): 267-273, 2000.
Sondermann, P et al, “Molecular basis for immune complex recognition: a comparison of Fc-receptor structures.” J Mol Biol, 309(3): 737-749, 2001.
Wallace, A C et al, “LIGPLOT: a program to generate schematic diagrams of protein-ligand interactions.” Protein Eng, 8(2): 127-134, 1995.
Warmerdam, P A et al, “Molecular basis for a polymorphism of human Fc gamma receptor II (CD32). ” J Exp Med, 172(1): 19-25, 1990.
Weiner S J et al, “A new force field for molecular mechanical simulation of nucleic acids and proteins”. J. Am. Chem. Soc, 106: 765-784, 1984.
Weng, W K and R Levy, “Two immunoglobulin G Fc receptor polymorphisms independently predict response to rituximab in patients with follicular lymphoma. ” J Clin Oncol, 21(21): 3940-3947, 2003.

1. A method for identifying an agent for modulating the biological activity of an Fc receptor protein (FcR), said method comprising the steps of:
(i) generating a three-dimensional structure model of high responder FcγRIIa (HR_{S88}) or a portion thereof, wherein said structure model comprises the three-dimensional structure of a target site with which an agent may interact and thereby modulate the biological activity of the receptor, and
(ii) identifying a candidate agent by designing or selecting a compound or chemical complex with a three-dimensional structure enabling interaction with said target site.

2. A method according to claim 1 wherein the method is for identifying a candidate agent for modulation of the interaction between the monomers of a dimer of HR_{S88}, said method comprising the steps of:
(i) generating a three-dimensional structure model of a dimer of HR_{S88} or a portion thereof in which portions of each monomer are represented, wherein said structure model comprises the three-dimensional structure of a target site with which an agent may interact and thereby modulate the interaction between the monomers; and
(ii) identifying a candidate agent by designing or selecting a compound or chemical complex with a three-dimensional structure enabling interaction with said target site.

3. A method for screening compounds and/or chemical complexes for a candidate agent for modulating the biological activity of an Fc receptor protein (FcR), said method comprising the steps of:
(i) generating a three-dimensional structure model of high responder FcγRIIa (HR_{S88}) or a portion thereof, wherein said structure model comprises the three-dimensional structure of a target site with which an agent may interact and thereby modulate the biological activity of the receptor, and
(ii) screening said compounds and/or chemical complexes to identify any compound(s) or chemical complex(es) having a three-dimensional structure which enables interaction with said target site.

4. A method according to claim 3 wherein the method is for screening compounds and/or chemical complexes for a candidate agent for modulation of the interaction between the monomers of a dimer of HR_{S88}, said method comprising the steps of:
(i) generating a three-dimensional structure model of a dimer of HR_{S88} or a portion thereof in which portions of each monomer are represented, wherein said structure model comprises the three-dimensional structure of a target site with which an agent may interact and thereby modulate the interaction between the monomers; and
(ii) screening said compounds and/or chemical complexes to identify any compound(s) or chemical complex(es) having a three-dimensional structure which enables interaction with said target site.

5. A method for modifying a candidate agent for modulating the biological activity of an Fc receptor protein (FcR), said method comprising the steps of:
(i) generating a three-dimensional structure model of high responder FcγRIIa (HR_{S88}) or a portion thereof, wherein said structure model comprises the three-dimensional structure of a target site with which an agent may interact and thereby modulate the biological activity of the receptor, and
(ii) modifying the candidate agent to provide an agent with a three-dimensional structure more favourable to providing a desired level of interaction with said target site than the candidate agent.

6. A method according to claim 5 wherein the method is for modifying a candidate agent for modulation of the interaction between the monomers of a dimer of HR_{S88} to provide an agent with improved activity, said method comprising the steps of:
(i) generating a three-dimensional structure model of a dimer of HR_{S88} or a portion thereof in which portions of each monomer are represented, wherein said structure model comprises the three-dimensional structure of a target site with which an agent may interact and thereby modulate the interaction between the monomers; and
(ii) modifying the candidate agent to provide an agent with a three-dimensional structure more favourable to providing a desired level of interaction with said target site than the candidate agent.

7. A method according to claim 1 wherein the target site is a surface on HR_{S88} selected from the group consisting of:
(a) the surface forming the immunoglobulin-binding site;
(b) the surface forming the dimerisation interface between two HR_{S88} monomers of a dimerised receptor;
(c) the surface forming a large groove between two HR_{S88} monomers of a dimerised receptor (site A); and
(d) the surface forming a cavity, channel and two identical pockets adjacent to the dimerisation interface between two HR_{S88} monomers of a dimerised receptor (site B).

8. A method according to claim 7 wherein the target site is the immunoglobulin-binding site and the surface of immunoglobulin-binding site comprises a structure defined by the conformation of amino acid residues 113-116, 129, 131, 133, 134, 155, 156 and 158-160.

9. A method according to claim 7 wherein the target site is the dimerisation interface and the surface of the dimerisation interface comprises a structure defined by the conformation of amino acid residues 26, 33, 54-56, 58, 102, 103, 105, 142 and 143 of one monomer of the HR_{S88} dimer and the equivalent residues of the other monomer of the dimer.

10. A method according to claim 7 wherein the target site is site A of an HR_{S88} dimer and the surface of site A comprises a structure defined by the conformation of amino acid residues 22-24, 60, 107, 109, 110, 112, 114-118, 131, 133-138, 140 and 160 of one monomer of the HR_{S88} dimer and the equivalent residues of the other monomer of the dimer.

11. A method according to claim 7 wherein the target site is site B of an HR_{S88} dimer and the surface of site B comprises a structure defined by the conformation of amino acid residues 12-16, 26, 96, 100 and 105 of one monomer of the HR_{S88} dimer and the equivalent residues of the other monomer of the dimer.

12. A method of designing a variant of high responder FcγRIIa (HR_{S88}) with altered biological activity, said method comprising the steps of:
(i) generating a three-dimensional structure model of HR_{S88} or a portion thereof; and
(ii) modifying the model to provide a variant of HR_{S88} with altered biological activity.

13. The method of claim 12 wherein the method is for designing a variant of a dimer of HR_{S88} with altered biological activity, said method comprising the steps of;
(i) generating a three-dimensional structure model of a dimer of HR_{S88} or a portion thereof in which portions of each monomer are represented; and
(ii) modifying the model to provide a variant of the dimer of HR_{S88} with altered biological activity.

14. A method according to claim 1 wherein the three-dimensional structure model is generated using at least the atomic coordinate data of Table 3.

15. A method according to claim 3 wherein the three-dimensional structure model is generated using at least the atomic coordinate data of Table 3.

16. A method according to claim 5 wherein the three-dimensional structure model is generated using at least the atomic coordinate data of Table 3.

17. A method according to claim 12 wherein the three-dimensional structure model is generated using at least the atomic coordinate data of Table 3.

18. A method according to claim 9 wherein a dimer of HR_{S88} is generated by applying the symmetry operations of space group C222₁ to the atomic coordinates of Table 3.

19. A method according to claim 1 wherein the method is an in silico method.

20. A computer for producing a three-dimensional structure model of high responder FcγRIIa (HR_{S88}) or a portion thereof, said structure model comprising the three-dimensional structure of a target site to which an agent may interact and thereby modulate the activity of an Fc receptor protein (FcR), wherein said computer comprises:
(i) a machine-readable data storage medium comprising the atomic coordinate data of Table 3;
(ii) a working memory for storing instructions for processing said atomic coordinate data contained on the machine-readable data storage medium;
(iii) a central processing unit coupled to said working memory and to said machine-readable data storage medium for processing said atomic coordinate data to generate said three-dimensional structure model; and
(iv) a display coupled to said central processing unit for displaying a representation of said three-dimensional structure model.

21. A computer according to claim 20, further comprising:
(v) means for receiving and storing atomic coordinate data for a range of chemical components and substituents, wherein the central processing unit is capable of interacting with said receiving and storing means and selects from said range of chemical components and substituents suitable chemical components and substituents to assemble a compound or chemical complex which, based upon a three-dimensional structure generated by said central processing unit, a representation of which may be provided on said display simultaneously with the representation of said three-dimensional structural model of HR_{S88} protein or a portion thereof, is capable of interaction with said target site; and/or
(vi) means for receiving and storing atomic coordinate data for a range of compounds and/or chemical complexes, wherein the central processing unit is capable of interacting with said receiving and storing means to generate a three-dimensional structure for a compound or chemical complex selected from the range of compounds and/or chemical complexes, provide a representation of said three-dimensional structure on said display simultaneously with the representation of said three-dimensional structural model of HR_{S88} or a portion thereof, and thereby enable an assessment of whether said selected compound or chemical complex is capable of interaction with said target site.

22. A machine-readable data storage medium comprising the atomic coordinate data of Table 3.

23. A candidate agent identified in accordance with the method any claim 1.

24. An agent produced in accordance with the method of claim 5.

25. A variant of a high responder FcγRIIa (HR_{S88}) designed in accordance with claim 12.

26. Use of a candidate agent according to claim 23 in the preparation of a medicament for modulating the biological activity of FcR in a subject.

27. Use according to claim 26, wherein the FcR is FcγRIIa.

28. A method of modulating the biological activity of FcR in a subject in a subject, said method comprising administering to the subject a medicament comprising a candidate agent according to claim 23.

29. A method of producing a medicament, wherein said method comprises:
(i) identifying an agent in accordance with the method of claim 1, identifying a compound(s) and/or chemical complex(es) in accordance with the method of claim 3, or modifying a candidate agent in accordance with the method of claim to provide a modified agent,
(ii) chemically synthesising said agent, compound(s) and/or chemical complex(es) or modified agent,
(iii) evaluating the ability of the synthesised agent, compound(s) and/or chemical complex(es) or modified agent to treat an Fc receptor-mediated disease or condition, and
(iv) formulating the synthesised agent, compound(s) and/or chemical complex(es) or modified agent with a suitable, pharmaceutically-acceptable delivery vehicle or adjuvant to produce said medicament.

30. A method of treating an Fc receptor-mediated disease or condition in a subject, said method comprising administering to said subject a pharmaceutically-effective amount of an agent or a variant of HR_{S88} which binds to a surface on an Fc receptor (FcR) selected from:
(a) the surface forming the immunoglobulin-binding site;
(b) the surface forming the dimerisation interface between two HR_{S88} monomers of a dimerised receptor;
(c) the surface forming a large groove between two HR_{S88} monomers of a dimerised receptor (site A); and
(d) the surface forming a cavity, channel and two identical pockets adjacent to the dimerisation interface between two HR_{S88} monomers of a dimerised receptor (site B).

31. A method according to claim 30 wherein the target site is the immunoglobulin-binding site and the surface of immunoglobulin-binding site comprises a structure defined by the conformation of amino acid residues 113-116, 129, 131, 133, 134, 155, 156 and 158-160.

32. A method according to claim 30 wherein the target site is the dimerisation interface and the surface of the dimerisation interface comprises a structure defined by the conformation of amino acid residues 26, 33, 54-56, 58, 102, 103, 105, 142 and 143 of one monomer of the HR_{S88} dimer and the equivalent residues of the other monomer of the dimer.

33. A method according to claim 30 wherein the target site is site A of an HR_{S88} dimer and the surface of site A comprises a structure defined by the conformation of amino acid residues 22-24, 60, 107, 109, 110, 112, 114-118, 131, 133-138, 140 and 160 of one monomer of the HR_{S88} dimer and the equivalent residues of the other monomer of the dimer.

34. A method according to claim 30 wherein the target site is site B of an HR_{S88} dimer and the surface of site B comprises a structure defined by the conformation of amino acid residues 12-16, 26, 96, 100 and 105 of one monomer of the HR_{S88} dimer and the equivalent residues of the other monomer of the dimer.
